(19) United States
(12) Patent Application Publication
(10) Pub. No.: US 2023/0025807 A1
(43) Pub. Date: Jan. 26, 2023

(54) CD38 INHIBITORS

(71) Applicant: Mitobridge Inc., Cambridge, MA (US)

(72) Inventors: Santosh S. Kulkarni, Bangalore (IN); Bharat Lagu, Acton, MA (US); Xinyuan Wu, Chestnut Hill, MA (US)

(21) Appl. No.: 17/772,493

(22) PCT Filed: Oct. 29, 2020
(86) PCT No.: PCT/US2020/057921

§ 371 Date: Apr. 27, 2022

Related U.S. Application Data
(60) Provisional application No. 62/927,809, filed on Oct. 30, 2019.

(51) Int. Cl. C07D 417/14; C07D 417/10; C07D 403/10; C07D 471/04

(52) U.S. Cl. CPC C07D 417/14; C07D 417/10; C07D 403/10; C07D 471/04

(57) ABSTRACT

The present invention is directed to a compound of Formula (I) or a pharmaceutically acceptable salt thereof. Compounds of Formula (I) are CD38 inhibitors, which can be used to treat a disease or condition in a subject that benefits from an increase in NAD⁺ or to treat a mitochondrial disorder in a subject. Such disease or condition is a muscle structure disorder, a neuronal activation disorder, a muscle fatigue disorder, a muscle mass disorder, a metabolic disease, a cancer, a vascular disease, an ocular vascular disease, a muscular eye disease, or a renal disease. The present description discloses the synthesis and characterisation of exemplary compounds as well as pharmacological data thereof (e.g. pages 30 to 135; examples 1 to 61; table). Such an exemplary compound is e.g. N-((1r,4r)-4-(2-methoxyethoxy) cyclohexyl)-5-(thiazol-5-yl)-1H-indole-7-carboxamide (II).

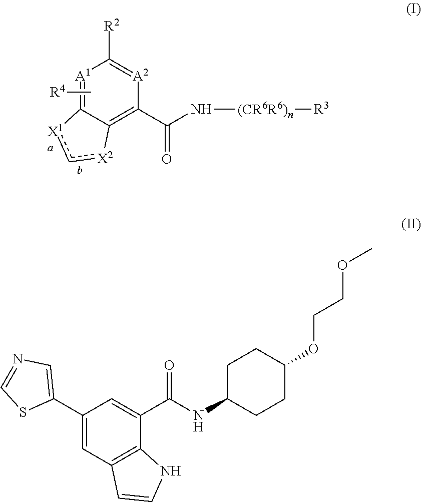

### RELATED APPLICATION

This application claims priority to U.S. Provisional Application No. 62/927,809, filed on Oct. 30, 2019, the entire contents of which are expressly incorporated herein by reference.

### FIELD OF THE INVENTION

This application is directed to CD38 inhibitors, and methods for their use, such as to control the activity of CD38 in a subject.

### BACKGROUND OF THE INVENTION

Nicotinamide Adenine Dinucleotide (NAD⁺) is a biochemical found in all cells that was first characterized over 100 years ago due to its role in oxidoreductase reactions. Since then, NAD⁺ and its related pyridine nucleotides NADH, NADP⁺, and NADPH are recognized as the major redox carriers in all organisms. These pyridine dinucleotides regulate the cytosolic and mitochondrial redox state and are key participants monitoring the metabolic status of the cell. This is because NAD⁺ and NADH act as hydride accepting and donating cofactors for metabolic enzymes involved in glycolysis, the TCA cycle, and the respiratory chain and thereby redistribute reducing equivalents generated from these catabolic processes into the de novo synthesis of new biomolecules. (Houtkooper et al Endo Reviews (2010) 31:194-223; Koch-Nolte et al Science Signaling (2009) 2:mr1; Houtkooper and Auwerx J. Cell Biol (2012) 199:205-209; Berger et al Trends in Bioch Sci (2004) 29:111-18)

In addition to its long recognized role as a cofactor for oxidoreductases, more recent research demonstrates that NAD⁺ is also a substrate for various enzymes, where it is consumed in the process of donating its ADP ribose to acceptor molecules. The enzymes that are the major consumers of NAD⁺ are the ADP ribosyl transferases (i.e., PARP and ART family of enzymes), the sirtuins (Sirt1-7), and the ADP ribosyl cyclases/hydrolases (CD38/CD157). These enzymes are involved in pathways that regulate Ca⁺⁺ signaling, gene transcription, DNA repair, cell survival, energy metabolism, and oxidative stress. Thus, NAD⁺ and its phosphorylated relatives NADP and NAADP, both of which are derived from NAD⁺, also act as signaling molecules. NAD⁺ is also a key component of the circadian cycle with daily oscillations that tie cellular metabolism to chromatin remodeling and gene transcription. It is known that exercise and caloric restriction elevate NAD⁺ levels, while aging and obesity decrease cellular NAD⁺ levels. Restoring NAD⁺ levels in disease states that consume significant amounts of NAD⁺ will likely have medical benefits as the cell strives to maintain its energy status during stress. (Tevy et al Trends in Endo and Metab (2013) 24:229-237; Pugh et al Aging Cell (2013) 12:672-681; Massudi et al PLoS ONE (2012) 7:e42357; Xu and Sauve (2010) Mech of Ageing and Development 131:287-298).

Cellular NAD⁺ is produced by either the de novo synthesis pathway from tryptophan or by a salvage synthesis pathway from precursors such as nicotinic acid (niacin) and nicotinamide, both of which are obtained from dietary sources. A third way to modulate cellular NAD+ levels is to block consumption of NAD+ by inhibiting enzymes that consume NAD+.

CD38 is one such consumer of NAD+. Also known as ADP ribosyl cyclase, CD38 is a type II membrane-anchored enzyme. It efficiently catalyzes the breakdown of NAD+ to nicotinamide and ADPR and hydrolyzes NAADP to ADPRP. CD38 can also act as a cyclase converting NAD+ to cADPR, although it is 100-fold less efficient as a cyclase than as a hydrolase. CD38 was first characterized as a surface antigen on immune cells and is broadly distributed throughout most tissues in the body. It exists on the plasma membrane and on the membranes of intracellular organelles such as the nucleus and mitochondria. As predicted from its function as a NAD+ glycohydrolase, CD38 KO mice have elevated NAD+ levels relative to wild-type controls. Likewise, inhibitors of CD38 enzyme activity also modulate NAD+ tissue levels and would be useful in treating various diseases where CD38 is over expressed or where cellular NAD+ levels are depressed or desynchronized. (Malavasi et al (2008) 88:841-886)

Compounds which inhibit CD38 and thereby raise NAD+ levels are useful in treating diseases or conditions indicated to benefit from NAD+ including mitochondrial-related diseases or disorders. Diseases which can be treated by raising NAD+ levels are disclosed in WO2016/087975 and WO2017/079195.

### SUMMARY OF THE INVENTION

Provided herein are compounds and compositions which inhibit CD38, thereby increasing the amount of NAD⁺ in cells. For example, the IC₅₀ values for inhibition of CD38 provided in Example 61 demonstrate that these compounds are potent inhibitors of CD38. Also disclosed are methods of using the disclosed compounds and compositions for treating mitochondrial-related diseases or disorders and disease which can benefit from increasing NAD⁺ levels in cells.

One embodiment of the invention is a compound represented by Formula I:

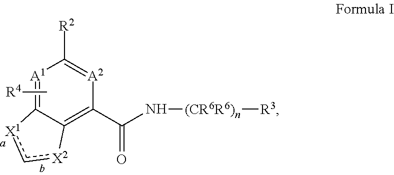

or a pharmaceutically acceptable salt thereof,
wherein:

A¹ and A² are independently CH or N, provided that A¹ and A² are not both N;

X¹ is CR^{1A} and X² is NR^{5A}, when bond a is a double bond and bond b is a single bond; or X¹ is NR^{1B} and X² is CR^{5B}, when bond a is a single bond and bond b is a double bond;

R^{1A} is H, C₁₋₄alkyl, NO₂, CN, CONR^{a}R^{b}, CH₂NR^{a}R^{b}, (CHR^{c})ₘOH, C₁₋₄haloalkyl, CHO, COOR^{a}, or halo;

R^{a}, R^{b} and R^{c} are each independently H or C₁₋₄alkyl;

R^{1B} is H or C₁₋₄alkyl optionally substituted with 3-5 membered monocyclic heterocyclyl or hydroxy;

R^{5A} is H or C₁₋₄alkyl;

R^{5B} is H, halo, CN, C₁₋₄alkyl, C₁₋₄haloalkyl, NHR^{b} or CONHR^{c};

R² is 5-membered heteroaryl;

R³ is C₁₋₄alkyl, C₃₋₆cycloalkyl, bridged C₇₋₁₂cycloalkyl, 5-6 membered monocyclic heterocyclyl optionally substituted with one or two oxo groups, or phenyl, wherein said alkyl, cycloalkyl, bridged cycloalkyl, heterocyclyl or phenyl is optionally substituted with one or two R^{x} groups, wherein R^{x} is halo, 3 to 6-membered heterocyclyl, C₁₋₄alkyl, C₁₋₄haloalkyl, C₁₋₄hydroxyalkyl, SO₂Me or OR^{d};

R^{d} is H or C₁₋₄alkyl optionally substituted with C₁₋₄alkoxy;

R⁴ is H, halo, CN, C₁₋₄alkyl, C₁₋₄haloalkyl, NHR^{e} or CONHR^{f};

R^{e} and R^{f} are each independently H or C₁₋₄alkyl;

R⁶ is H or C₁₋₄alkyl;

n is 0 or 1; and

m is 1, 2 or 3,

provided that, when R^{1A} is H or C₁₋₄ alkyl; and R² is

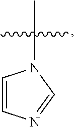 [SMILES: *n1ccnc1]

then n is 1; and
provided that when A¹ is N, X¹ is N and R^{1B} is C₁₋₄ alkyl, then n is 1; and, in an alternative, when A¹ is N; X¹ is N; R^{1B} is C₁₋₄ alkyl; and R³ is a optionally substituted phenyl, then n is 1.

Another embodiment of the invention is a pharmaceutical composition comprising a acceptable carrier or excipient and a compound disclosed herein or a pharmaceutically acceptable salt thereof.

Another embodiment of the invention is method of treating a disease or condition in a subject that benefits from an increase in NAD⁺ (or treating a mitochondrial disorder), comprising administering to the subject an effective amount of a compound disclosed herein or a pharmaceutically acceptable salt thereof or a pharmaceutical composition comprising the compound(s).

Another embodiment of the invention is the use of a compound disclosed herein or a pharmaceutically acceptable salt thereof or a pharmaceutical composition comprising the compound(s), for the preparation of a medicament for treating a disease or condition in a subject that benefits from an increase in NAD⁺ (or treating a mitochondrial disorder).

Another embodiment of the invention is a compound disclosed herein or a pharmaceutically acceptable salt thereof or a pharmaceutical composition comprising the compound(s), for use in treating a disease or condition in a subject that benefits from an increase in NAD⁺ (or treating a mitochondrial disorder).

### DETAILED DESCRIPTION

The disclosed compounds are CD38 inhibitors, which can be used for treating a disease or condition in a subject that benefits from an increase in NAD⁺ or for treating a mitochondrial disorder. Such diseases or disorders include a muscle structure disorder, a neuronal activation disorder, a muscle fatigue disorder, a muscle mass disorder, a metabolic disease, a cancer, a vascular disease, an ocular vascular disease, a muscular eye disease, or a renal disease. More specifically, a disease or condition for which a therapeutic benefit can be achieved from an increase in NAD⁺ (or a mitochondrial disorder) include non-alcoholic fatty liver disease (NAFLD), non-alcoholic steatohepatitis (NASH), renal ischemia/reperfusion injury (IRI), Duchenne & Becker muscular dystrophy, diabetes (type I or type II), obesity, and sarcopenia. In another embodiment, “a disease or condition for which a therapeutic benefit can be achieved from an increase in NAD⁺” or “a mitochondrial-related disease or disorder” is selected from Alpers's Disease, CPEO-Chronic progressive external ophthalmoplegia, Kearns-Sayra Syndrome (KSS), Leber Hereditary Optic Neuropathy (LHON), MELAS-Mitochondrial myopathy, encephalomyopathy, lactic acidosis, stroke-like episodes, MERRF-Myoclonic epilepsy and ragged-red fiber disease, NARP-neurogenic muscle weakness, ataxia, retinitis pigmentosa, Pearson Syndrome, platinum-based chemotherapy induced ototoxicity, Cockayne syndrome, xeroderma pigmentosum A, Wallerian degeneration, and HIV-induced lipodystrophy.

In a first embodiment, the invention is a compound represented by Formula I, or a pharmaceutically acceptable salt thereof. The variables in Formula I are described above. In one aspect, one of A¹ or A² is N; in another aspect A¹ is N; and in another aspect, A² is N.

In a second embodiment, the invention is a compound represented by Formula II:

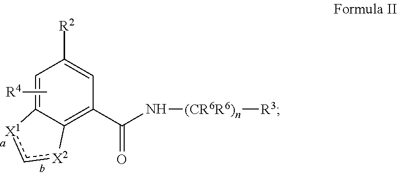

or a pharmaceutically acceptable salt thereof. The variables in Formula II are described above for Formula I.

In a third embodiment, the invention is a compound represented by Formula III or IV:

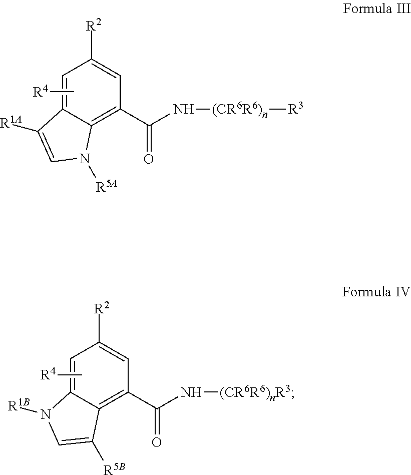

or a pharmaceutically acceptable salt thereof. The variables in Formulas III and IV are described above for Formula I.

In a fourth embodiment, the invention is a compound represented by Formula I, II, III and IV, or a pharmaceutically acceptable salt thereof,

wherein R² is

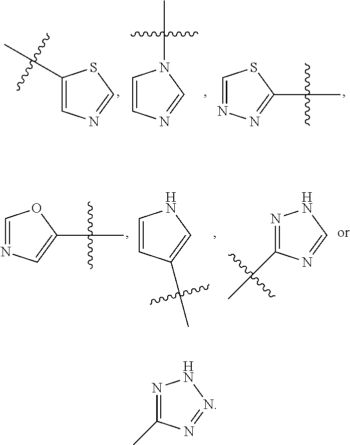

### Alternatively, R² is

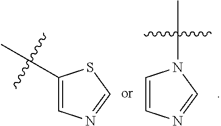

The remainder of the variables in Formulas 1, II, III and IV are described above for Formula I.

In a fifth embodiment, the invention is a compound represented by Formula I, II, III or IV, or a pharmaceutically acceptable salt thereof, wherein R³ is C₃₋₆cycloalkyl; 6-membered monocyclic heterocyclyl optionally substituted with one or two oxo groups; or bridged bicyclic C₉₋₁₁alkyl; wherein said C₃₋₆cycloalkyl, 6-membered monocyclic heterocyclyl or bridged bicyclic C₉₋₁₁alkyl is optionally substituted with one or two R^{x} groups; and n is 0. The remainder of the variables in Formulas 1, II, III or IV are described above in the first or the fourth embodiment.

In a sixth embodiment, the invention is a compound represented by Formula I, II, III or IV, or a pharmaceutically acceptable salt thereof, wherein R³ is C₃₋₆cycloalkyl or 6-membered monocyclic heterocyclyl optionally substituted with one R^{x} group. The remainder of the variables in Formulas 1, II, III or IV are described above in the first or the fourth embodiment.

In a seventh embodiment, the invention is a compound represented by Formula V or VI:

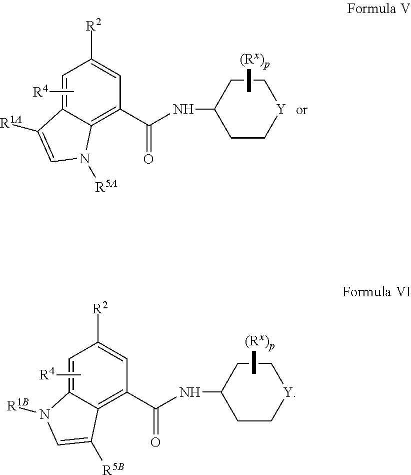

or a pharmaceutically acceptable salt thereof. Y is O, NH, SO₂, CH₂ or CHR^{x}; and p is 0 or 1; and the remainder of the variables in Formulas V and VI are described above in the first, fourth, fifth or sixth embodiment.

In an eighth embodiment, the invention is a compound represented by Formula VII or VIII:

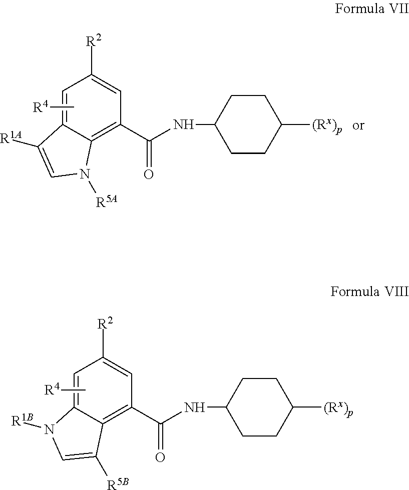

or a pharmaceutically acceptable salt thereof wherein p is 0 or 1. The variables in Formulas VII and VIII are described above in the first, fourth, fifth or sixth embodiment.

In a ninth embodiment, the invention is a compound represented by Formula IX or X:

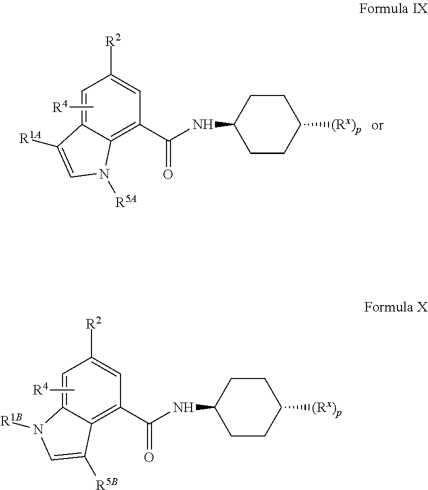

or a pharmaceutically acceptable salt thereof. The variables in Formulas IX and X are described above in the first, sixth or seventh embodiment. The remainder of the variables in Formulas I-X are described above in any one of the preceding embodiments.

In a tenth embodiment, the invention is a compound represented by any one of Formulas I-X, or a pharmaceutically acceptable salt thereof, wherein R^{x} is C₁₋₄hydroxyalkyl or OR^{d}. The remainder of the variables in Formulas I-X are described above in any one of the preceding embodiments.

In an eleventh embodiment, the invention is a compound represented by any one of Formulas I-X, or a pharmaceutically acceptable salt thereof, wherein R^{d} is H; alternatively, R^{d} is C₁₋₄alkyl substituted with C₁₋₄alkoxy. The remainder of the variables in Formulas I-X are described above in the tenth embodiment.

In a twelfth embodiment, the invention is a compound represented by any one of Formulas I-X, or a pharmaceutically acceptable salt thereof, wherein R^{x} is OH, OCH₂CH₂OMe or OCH₂CH₂CH₂OMe. The remainder of the variables in Formulas I-X are described above in any one of the preceding embodiments.

In a thirteenth embodiment, the invention is a compound represented by any one of Formulas I-IV, or a pharmaceutically acceptable salt thereof, wherein R³ is tetrahydro-2H-pyran or phenyl; and n is 1, wherein said phenyl is optionally substituted with one or two R^{x} groups. The remainder of the variables in Formulas I-IV are described above in the first, fifth, sixth embodiment.

In a fourteenth embodiment, the invention is a compound represented by any one of Formulas I-IV, or a pharmaceutically acceptable salt thereof, wherein R³ is phenyl, and R^{x} is halo, C₁₋₄haloalkyl or 6-membered heterocyclyl. The remainder of the variables in Formulas I-IV are described above in the thirteenth embodiment.

In a fifteenth embodiment, the compound of the invention is a compound is

represented by Formula XI or XII:

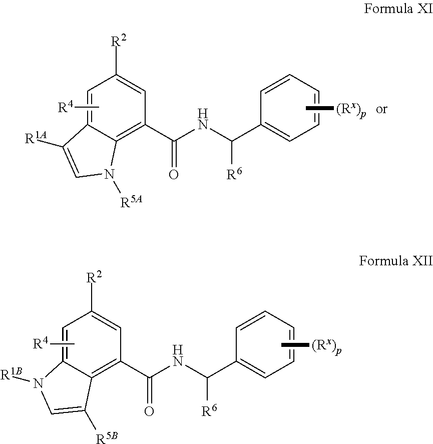

or a pharmaceutically acceptable salt thereof, wherein R^{x} is C₁₋₄haloalkyl, halo or 6-membered heterocyclyl; and wherein p is 1 or 2. Alternatively, p is 2, and one R^{x} is trifluoromethyl and another R^{x} is F; or p is 1, and R^{x} is F. The remainder of the variables in Structural Formulas XI and XII are as described in the thirteenth and fourteenth embodiments.

In a sixteenth embodiment, the invention is a compound represented by any one of Formulas I-IV, XI and XII, or a pharmaceutically acceptable salt thereof, wherein R⁶ is H or methyl. The remainder of the variables are as described in any one of the first through the thirteenth, fourteenth or fifteenth embodiment.

In a seventeenth embodiment, the invention is a compound represented by any one of Formulas I-XII, or a pharmaceutically acceptable salt thereof, wherein R^{1A} is H, halo, NO₂, CN, COOR^{a}, CHO, CONR^{a}R^{b}, (CHR^{c})ₘOH, CH₂NR^{a}R^{b} or C₁₋₄haloalkyl; or wherein R^{1B} is H or C₁₋₃alkyl optionally substituted with 4-membered heterocyclyl or hydroxyl; and wherein R^{a} and R^{b} are independently H or methyl. The remainder of the variables are as described in any one of the of the previous embodiments. Alternatively, R^{1A} is (CHR^{c})ₘOH; R^{c} is H or methyl; and m is 1.

In an eighteenth embodiment, the invention is a compound represented by Formula any one of Formulas I-XII, or a pharmaceutically acceptable salt thereof, wherein R^{1A} is H, I, NO₂, CN, CH₂NH₂, CHO, COOH, COOMe, CH₂OH, CHOHMe, CH₂CH₂OH, CF₃, CONH₂, CONHMe or CONMe₂; or wherein R^{1B} is H, methyl, isopropyl, hydroxyisobutyl or oxetylmethyl. The remainder of the variables are as described in the seventeenth embodiment.

In a nineteenth embodiment, the invention is a compound represented by Formula any one of Formulas I-XII, or a pharmaceutically acceptable salt thereof, wherein R⁴ is H or halo (preferably F). The remainder of the variables are as described in any one of the of the previous embodiments.

In a nineteenth embodiment, the invention is a compound represented by Formula any one of Formulas I-XII, or a pharmaceutically acceptable salt thereof, wherein R^{5A} is H or methyl; or wherein R^{5B} is H, methyl or CN. The remainder of the variables are as described in any one of the of the previous embodiments.

The invention also includes the compounds depicted in the Table and prepared in the Exemplification. Both pharmaceutically acceptable salts and the neutral form of these compounds are included.

Included in the present teachings are pharmaceutically acceptable salts of the compounds disclosed herein. Compounds of the present teachings with basic groups can form pharmaceutically acceptable salts with pharmaceutically acceptable acid(s). Suitable pharmaceutically acceptable acid addition salts of the compounds described herein include salts of inorganic acids (such as hydrochloric acid, hydrobromic, phosphoric, nitric, and sulfuric acids) and of organic acids (such as acetic acid, benzenesulfonic, benzoic, methanesulfonic, and p-toluenesulfonic acids). Compounds of the present teachings with acidic groups can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically-acceptable salts are well known in the art. For example, S. M. Berge et al. describes pharmacologically acceptable salts in J. Pharm. Sci. (1977) 66:1-19.

The term “alkyl” used alone or as part of a larger moiety, such as “alkoxy”, “haloalkyl”, “hydroxyalkyl” and the like, means a saturated aliphatic straight-chain or branched monovalent hydrocarbon radical. Unless otherwise specified, an alkyl group typically has 1 to 6 carbon atoms, i.e., C₁-C₆-alkyl. As used herein, a “C₁-C₆-alkyl” group is means a radical having from 1 to 6 carbon atoms in a linear or branched arrangement, such as methyl, ethyl, propyl, isopropyl, butyl, isobutyl, tert-butyl, and the like

“Alkoxy” means an alkyl radical attached through an oxygen linking atom, represented by —O-alkyl. For example, “C₁-C₆-alkoxy” includes methoxy, ethoxy, propoxy, butoxy, pentoxy, isopentoxy, isopropoxy, and hexoxy.

The terms “haloalkyl” and “haloalkoxy” mean alkyl or alkoxy, as the case may be, substituted with one or more halogen atoms. In some embodiments, “haloalkyl” and “haloalkoxy” means alkyl or alkoxy, as the case may be, substituted with one or more fluorine atoms.

The term “halogen” means fluorine or fluoro (F), chlorine or chloro (Cl), bromine or bromo (Br), or iodine or iodo (I).

“Cycloalkyl” means a saturated aliphatic cyclic hydrocarbon radical. It can be monocyclic, bicyclic (e.g., a bridged bicyclic ring), polycyclic (e.g., tricyclic), or fused. Unless otherwise specified, a cycloalkyl has 3-12 ring carbon atoms, alternatively 3-6 ring carbon atoms. For example, “C₃-C₆-cycloalkyl” means a radical having from 3 to 6 carbon atoms arranged in a monocyclic ring. A C₃-C₆-cycloalkyl includes, but is not limited to, cyclopropyl, cyclobutyl, cyclopentyl, and cyclohexyl. Bridged cycloalkyl groups have unless otherwise specified 7-12 ring carbon atoms. Examples include adamantyl, bicyclo[3.3.1]nonane, bicyclo[2.2.2]octane, bicyclo[2.2.1]heptane and the like.

The term “fused” refers to two rings that share two adjacent ring ring atoms with one another.

The term “bridged” refers to two rings that share three adjacent ring atoms with one another.

The term “heteroaryl”, “heteroaromatic”, “heteroaryl ring”, “heteroaryl group”, “heteroaromatic ring”, and “heteroaromatic group”, used alone or as part of a larger moiety as in “heteroaralkyl” or “heteroarylalkoxy”, refers to monocyclic aromatic ring groups having five or six ring atoms (i.e., “5-6 membered”) selected from carbon and at least one (typically 1 to 4, more typically 1 or 2) heteroatoms (e.g., oxygen, nitrogen or sulfur).

Examples of monocyclic heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl), thienyl (e.g., 2-thienyl, 3-thienyl), pyrimidinyl, pyridinyl and pyridazinyl.

The term “heterocyclyl” refers to a monocyclic non-aromatic ring radical containing from 3-6 ring atoms (i.e., “3-6 membered”) selected from carbon atom and 1 or 2 heteroatoms. Each heteroatom is independently selected from nitrogen, quaternary nitrogen, oxidized nitrogen (e.g., NO); oxygen; and sulfur, including sulfoxide and sulfone. Representative heterocyclyl groups include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A “substituted heterocyclyl group” is substituted at any one or more substitutable ring atom, which is a ring carbon or ring nitrogen atom bonded to a hydrogen.

If a group is described as being “substituted”, a non-hydrogen substituent is in the place of a hydrogen substituent on a carbon, sulfur or nitrogen of the group. Thus, for example, a substituted alkyl is an alkyl wherein at least one non-hydrogen substituent is in the place of a hydrogen substituent on the alkyl group. To illustrate, monofluoroalkyl is an alkyl substituted with a fluoro substituent, and difluoroalkyl is an alkyl substituted with two fluoro substituents. It should be recognized that if there is more than one substitution on a substituent, each non-hydrogen substituent can be identical or different (unless otherwise stated).

If a group is described as being “optionally substituted”, the substituent can be either (1) not substituted, or (2) substituted.

If a group is described as being optionally substituted with up to a particular number of non-hydrogen substituents, that group can be either (1) not substituted; or (2) substituted by up to that particular number of non-hydrogen substituents or by up to the maximum number of substitutable positions on the substituent, whichever is less. Thus, for example, if a group is described as a cycloalkyl optionally substituted with up to 3 non-hydrogen substituents, then any cycloalkyl with less than 3 substitutable positions would be optionally substituted by up to only as many non-hydrogen substituents as the cycloalkyl has substitutable positions.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A“geometric isomer” refers to isomers that differ in the orientation of substituent group in relationship to a cycloalkyl or heterocycle ring, i.e., cis or trans isomers. “Cis” refers to substituents oriented on the same side of the ring, whereas trans refers to substituents oriented on opposite sides of the ring.

When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer or a stereoisomer is depicted by name or structure, it is to be understood that the named or depicted isomer exists to a greater degree than its corresponding isomer, that is that the geometric isomeric purity of the named or depicted geometric isomer is greater than 50%, such as at least 60%, 70%, 80%, 90%, 99% or 99.9% pure by weight. Geometric isomeric purity is determined by dividing the weight of the named or depicted geometric isomer in the mixture by the total weight of all of the geometric isomers in the mixture.

Racemic mixture means 50% of one enantiomer and 50% of its corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures if one or more diastereomers e.g., racemic mixtures) of the compound.

Enantiomeric mixtures can be resolved into their component enantiomers by well-known methods, such as chiral-phase gas chromatography, chiral-phase high performance liquid chromatography, crystallizing the compound as a chiral salt complex, or crystallizing the compound in a chiral solvent. Enantiomers also can be obtained from enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers is at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

Methods of treating a mitochondria-related disease or condition in a subject are disclosed. Also disclosed herein are methods of treating a disease or disorder that would benefit from increased NAD⁺ levels, for example by increasing in vivo levels of NAD⁺ (e.g. intracellular NAD⁺ levels, levels of NAD⁺ in tissues or plasma, and/or overall NAD⁺ levels in an organism). The methods can include administering to the subject an effective amount of one or more compounds or compositions provided herein.

Diseases and disorders that can be treated by the disclosed compounds and pharmaceutical compositions include diseases or disorders related to aging or stress, diabetes, obesity, neurodegenerative diseases, cardiovascular disease, blood clotting disorders, inflammation, cancer, and/or flushing, etc.

In one embodiment, the diseases or disorders includes, but is not limited to, Alpers's Disease, CPEO-Chronic progressive external ophthalmoplegia, Kearns-Sayra Syndrome (KSS), Leber Hereditary Optic Neuropathy (LHON), MELAS-Mitochondrial myopathy, encephalomyopathy, lactic acidosis, stroke-like episodes, MERRF-Myoclonic epilepsy and ragged-red fiber disease, NARP-neurogenic muscle weakness, ataxia, retinitis pigmentosa, Pearson Syndrome, platinum-based chemotherapy induced ototoxicity, Cockayne syndrome, xeroderma pigmentosum A, Wallerian degeneration, and HIV-induced lipodystrophy.

In one embodiment, the mitochondrial-related disease or disorder or disease or disorder that would benefit from increased NAD⁺ levels is a muscle structure disorder, a neuronal activation disorder, a muscle fatigue disorder, a muscle mass disorder, a metabolic disease, a cancer, a vascular disease, an ocular vascular disease, a muscular eye disease, or a renal disease.

The muscle structure disorder is selected from Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorders, myotonic chondrodystrophy, myotonic dystrophy, myotubular myopathy, nemaline body disease, oculopharyngeal MD, and stress urinary incontinence.

The neuronal activation disorder is selected from amyotrophic lateral sclerosis, Charcot-Marie-Tooth disease, Guillain-Barre syndrome, Lambert-Eaton syndrome, multiple sclerosis, myasthenia gravis, nerve lesion, peripheral neuropathy, spinal muscular atrophy, tardy ulnar nerve palsy, and toxic myoneural disorder.

The muscle fatigue disorder is selected from chronic fatigue syndrome, diabetes (type I or II), glycogen storage disease, fibromyalgia, Friedreich's ataxia, intermittent claudication, lipid storage myopathy, MELAS, mucopolysaccharidosis, Pompe disease, and thyrotoxic myopathy; the muscle mass disorder is selected from cachexia, cartilage degeneration, cerebral palsy, compartment syndrome, critical illness myopathy, inclusion body myositis, muscular atrophy (disuse), sarcopenia, steroid myopathy, and systemic lupus erythematosus.

The beta oxidation disease is selected from systemic carnitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, and riboflavin-responsive disorders of β-oxidation (RR-MADD).

The metabolic disease is selected from hyperlipidemia, dyslipidemia, hyperchlolesterolemia, hypertriglyceridemia, HDL hypocholesterolemia, LDL hypercholesterolemia and/or HLD non-cholesterolemia, VLDL hyperproteinemia, dyslipoproteinemia, apolipoprotein A-I hypoproteinemia, atherosclerosis, disease of arterial sclerosis, disease of cardiovascular systems, cerebrovascular disease, peripheral circulatory disease, metabolic syndrome, syndrome X, obesity, diabetes (type I or II), hyperglycemia, insulin resistance, impaired glucose tolerance, hyperinsulinism, diabetic complication, cardiac insufficiency, cardiac infarction, cardiomyopathy, hypertension, Non-alcoholic fatty liver disease (NAFLD), Nonalcoholic steatohepatitis (NASH), thrombus, Alzheimer disease, neurodegenerative disease, demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, and pancreatitis.

The vascular disease is selected from peripheral vascular insufficiency, peripheral vascular disease, intermittent claudication, peripheral vascular disease (PVD), peripheral artery disease (PAD), peripheral artery occlusive disease (PAOD), and peripheral obliterative arteriopathy.

The ocular vascular disease is selected from age-related macular degeneration (AMD), stargardt disease, hypertensive retinopathy, diabetic retinopathy, retinopathy, macular degeneration, retinal haemorrhage, and glaucoma.

The muscular eye disease is selected from strabismus, progressive external ophthalmoplegia, esotropia, exotropia, a disorder of refraction and accommodation, hypermetropia, myopia, astigmatism, anisometropia, presbyopia, disorders of accommodation, and internal ophthalmoplegia.

The renal disease is selected from glomerulonephritis, glomerulosclerosis, nephrotic syndrome, hypertensive nephrosclerosis, acute nephritis, recurrent hematuria, persistent hematuria, chronic nephritis, rapidly progressive nephritis, acute renal failure (also known as acute kidney injury), chronic renal failure, diabetic nephropathy, and Bartter's syndrome.

In another embodiment, the mitochondrial-related disease or condition or disease or disorder that would benefit from increased NAD⁺ levels is selected from genetic lipodystrophy, non-alcoholic fatty liver disease (NAFLD), non-alcoholic steatohepatitis (NASH), renal ischemia/reperfusion injury (IRI), Duchenne & Becker muscular dystrophy, diabetes (type I or type II), obesity, and sarcopenia.

In another embodiment, the compounds of the invention and pharmaceutical compositions thereof may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated using the compounds of the invention and pharmaceutical compositions thereof prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with the CD38 inhibitor preparations or pharmaceutical compositions of the invention, or may have a subset of cells/tissue treated locally with the compounds of the invention and pharmaceutical compositions thereof. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, the compounds of the invention and/or a pharmaceutical composition thereof can be used to treat skin conditions. Exemplary skin conditions that may be treated in accordance with the methods described herein include disorders or diseases associated with or caused by inflammation, sun damage or natural aging. For example, the compositions find utility in the treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), scleroderma, damage caused by the sun or other light sources, discoid lupus erythematosus, dermatomyositis, psoriasis, skin cancer and the effects of natural aging. In another embodiment, the compounds of the invention and pharmaceutical compositions thereof may be used for the treatment of wounds and/or burns to promote healing, including, for example, first-, second- or third-degree burns and/or thermal, chemical or electrical burns.

The compounds of the invention and pharmaceutical compositions thereof can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amyotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

The compounds of the invention and pharmaceutical compositions thereof can also be administered to a subject suffering from an acute disease, e.g., damage to an organ or tissue, e.g., a subject suffering from stroke or myocardial infarction or a subject suffering from a spinal cord injury. The compounds of the invention and pharmaceutical compositions thereof may also be used to repair an alcoholic's liver.

In another embodiment, the invention provides a method for treating a cardiovascular disease by administering to a subject in need thereof one or more of the compounds of the invention and/or a pharmaceutical composition thereof. Cardiovascular diseases that can be treated using the compounds of the invention and pharmaceutical compositions thereof include cardiomyopathy or myocarditis, such as idiopathic cardiomyopathy, metabolic cardiomyopathy, alcoholic cardiomyopathy, drug-induced cardiomyopathy, ischemic cardiomyopathy, complications associated with percutaneous coronary intervention, and hypertensive cardiomyopathy. Also treatable using compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated include those related to platelet aggregation, the retinal arterioles, the glomerular arterioles, the vasa nervorum, cardiac arterioles, and associated capillary beds of the eye, the kidney, the heart, and the central and peripheral nervous systems. The compounds of the invention and pharmaceutical compositions thereof may also be used for increasing HDL levels in plasma of an individual.

The compounds of the invention and pharmaceutical compositions thereof may be administered to subjects who have recently received or are likely to receive a dose of radiation or toxin. In one embodiment, the dose of radiation or toxin is received as part of a work-related or medical procedure, e.g., working in a nuclear power plant, flying an airplane, an X-ray, CAT scan, or the administration of a radioactive dye for medical imaging; in such an embodiment, the compound is administered as a prophylactic measure. In another embodiment, the radiation or toxin exposure is received unintentionally, e.g., as a result of an industrial accident, habitation in a location of natural radiation, terrorist act, or act of war involving radioactive or toxic material. In such a case, the compounds of the invention and pharmaceutical compositions thereof is preferably administered as soon as possible after the exposure to inhibit apoptosis and the subsequent development of acute radiation syndrome.

In another embodiment, the compounds of the invention and pharmaceutical compositions thereof may be useful for treating age-related disorders, such as, for example, cancer.

Exemplary cancers that may be treated using the compounds of the invention and pharmaceutical compositions thereof include those of the brain and kidney; hormone-dependent cancers including breast, prostate, colon, large intestine, skin, lung, testicular, pancreas, and ovarian cancers; lymphomas, and leukemias. Other diseases that can be treated include autoimmune diseases, e.g., systemic lupus erythematosus, systemic scleroderma, and arthritis, in which autoimmune cells should be removed. Viral infections such as herpes, HIV, adenovirus, and HTLV-1 associated malignant and benign disorders can also be treated by administration of one or more of the compounds of the invention and pharmaceutical compositions thereof.

In another embodiment, the compounds of the invention and pharmaceutical compositions thereof may be useful for accelerated aging disorders, such as, for example, Hutchinson-Gilford progeria syndrome, Werner syndrome, telomere deficiency syndromes or telomeropathies, and dyskeratosis gongenita. Additional diseases that can be treated include those associated with stress-induced premature senescence, which may include extrapulmonary complications of COPD such as cardiovascular disease, osteoporosis, and dementia, and diseases where “inflammaging” or secreted factors from senescent cells alter the balance of NAD synthesis and consumption.

In certain aspects, the compounds of the invention and pharmaceutical compositions thereof can be used to treat patients suffering from neurodegenerative diseases, and traumatic or mechanical injury to the central nervous system (CNS) or peripheral nervous system (PNS). Examples of neurodegenerative diseases include, but are not limited to, Alzheimer's disease (AD), Parkinson's disease (PD), Huntington disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. The compounds of the invention and pharmaceutical compositions thereof can be used to treat these disorders and others as described below.

In an exemplary embodiment, the compounds of the invention and pharmaceutical compositions thereof may be used to treat multiple sclerosis (MS), including relapsing MS and monosymptomatic MS, and other demyelinating conditions, such as, for example, chromic inflammatory demyelinating polyneuropathy (CIDP), or symptoms associated therewith.

In yet another embodiment, the compounds of the invention and pharmaceutical compositions thereof may be used to treat trauma to the nerves, including, trauma due to disease, injury (including surgical intervention), or environmental trauma (e.g., neurotoxins, alcoholism, etc.).

The compounds of the invention and pharmaceutical compositions thereof may also be useful to treat and alleviate symptoms of various peripheral nervous system (PNS) disorders. PNS disorders include a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used. PNS disorders may be the result of, for example, leprosy, diabetes, Guillain-Barre syndrome, and others.

Other PNS diseases treatable with the compounds of the invention and pharmaceutical compositions thereof include Brachial Plexus Neuropathies (diseases of the cervical and first thoracic roots, nerve trunks, cords, and peripheral nerve components of the brachial plexus). Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed. pp. 1351-2). Also included are Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy (see Adams et al., Principles of Neurology, 6th ed., p. 1325). PNS diseases also include mononeuropathies (disease or trauma involving a single peripheral nerve in isolation, or out of proportion to evidence of diffuse peripheral nerve dysfunction). Mononeuropathy multiplex refers to a condition characterized by multiple isolated nerve injuries. Mononeuropathies may result from a wide variety of causes, including ischemia; traumatic injury; compression; connective tissue diseases; cumulative trauma disorders; and other conditions. Also included are Neuralgia (intense or aching pain that occurs along the course or distribution of a peripheral or cranial nerve); Peripheral Nervous System Neoplasms (neoplasms which arise from peripheral nerve tissue, this includes neurofibromas; Schwannomas; granular cell tumors; and malignant peripheral nerve sheath tumors, see DeVita Jr et al., Cancer: Principles and Practice of Oncology, 5th ed, ppl 750-1); and Nerve Compression Syndromes (mechanical compression of nerves or nerve roots from internal or external causes, these may result in a conduction block to nerve impulses, due to, for example, myelin sheath dysfunction, or axonal loss; the nerve and nerve sheath injuries may be caused by ischemia; inflammation; or a direct mechanical effect); and Neuritis (a general term indicating inflammation of a peripheral or cranial nerve). Clinical manifestation may include pain; paresthesias; paresis; or hyperthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

The compounds of the invention and pharmaceutical compositions thereof can also be used to treat blood coagulation disorders (or hemostatic disorders). As used interchangeably herein, the terms “hemostasis”, “blood coagulation,” and “blood clotting” refer to the control of bleeding, including the physiological properties of vasoconstriction and coagulation.

The present invention also provides anticoagulation and antithrombotic treatments aiming at inhibiting the formation of blood clots in order to treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering the compounds of the invention and pharmaceutical compositions thereof. The compositions and methods disclosed herein are useful for the treatment of thrombotic disorders. As used herein, the term “thrombotic disorder” includes any disorder or condition characterized by excessive or unwanted coagulation or hemostatic activity, or a hypercoagulable state. Examples of thrombotic disorders include, but are not limited to, thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, e.g., unstable angina, disseminated intravascular coagulation, thrombotic thrombocytopenic purpura, cancer metastasis, sickle cell disease, glomerular nephritis, and drug induced thrombocytopenia (including, for example, heparin induced thrombocytopenia).

In addition, the compounds of the invention and pharmaceutical compositions thereof may be administered to reduce thrombotic events or to reduce re-occlusion during or after therapeutic clot lysis or procedures such as angioplasty or surgery.

The compounds of the invention and pharmaceutical compositions thereof may also be used for treating or reducing weight gain or obesity in a subject. For example, the compounds of the invention and pharmaceutical compositions thereof may be used to treat hereditary obesity, dietary obesity, hormone related obesity, obesity related to the administration of medication, to reduce the weight of a subject, or to reduce weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, the compounds of the invention and pharmaceutical compositions thereof may be administered to subjects suffering from a variety of other diseases and conditions that may be treated by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholecystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self-esteem). Stunkard A J, Wadden T A. (Editors) Obesity: theory and therapy, Second Edition. New York: Raven Press, 1993. Finally, patients with AIDS can develop lipodystrophy or insulin resistance in response to combination therapies for AIDS. In another embodiment, the compounds of the invention and pharmaceutical compositions thereof may be used for inhibiting adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used for treating obesity.

In other embodiments, the compounds of the invention and pharmaceutical compositions thereof may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

In other embodiments, the compounds of the invention and pharmaceutical compositions thereof may be used to treat a subject who has cachexia or may be likely to develop cachexia. A method may further comprise monitoring in the subject the state of the disease. Methods for promoting appetite and/or weight gain may include, for example, prior identifying a subject as being in need of decreased fat or lipid metabolism, e.g., by weighing the subject, determining the BMI of the subject. The method may also include monitoring the subject, e.g., during and/or after administration of the compounds of the invention and pharmaceutical compositions thereof. The administering can include one or more dosages, e.g., delivered in boluses or continuously. Monitoring can include evaluating a hormone or a metabolite. Exemplary hormones include leptin, adiponectin, resistin, and insulin. Exemplary metabolites include triglycerides, cholesterol, and fatty acids.

In another embodiment, the compounds of the invention and pharmaceutical compositions thereof may be administered to reduce drug-induced weight gain. For example, the compounds of the invention and pharmaceutical compositions thereof may be administered as a combination therapy with medications that may stimulate appetite or cause weight gain, in particular, weight gain due to factors other than water retention.

Examples of medications that may cause weight gain, include for example, diabetes treatments, including, for example, sulfonylureas (such as glipizide and glyburide), thiazolidinediones (such as pioglitazone and rosiglitazone), meglitinides, nateglinide, repaglinide, sulphonylurea medicines, and insulin; anti-depressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy; lithium carbonate; valproic acid; carbamazepine; chlorpromazine; thiothixene; beta blockers (such as propranolol); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, the compounds of the invention and pharmaceutical compositions thereof may be administered as part of a smoking cessation program to reduce weight gain or reduce weight already gained.

In another aspect, the compounds of the invention and pharmaceutical compositions thereof may be used for treating a metabolic disorder, such as insulin-resistance, a pre-diabetic state, type II diabetes, and/or complications thereof.

Administration of the compounds of the invention and pharmaceutical compositions thereof may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

The compounds of the invention and pharmaceutical compositions thereof can also be used to treat a disease or disorder associated with inflammation. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatitis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components.

In another embodiment, the compounds of the invention and pharmaceutical compositions thereof may be used to treat allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, the compounds of the invention and pharmaceutical compositions thereof may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosus, Addison's disease, autoimmune polyglandular disease (also known as autoimmune polyglandular syndrome), and Grave's disease.

The compounds of the invention and pharmaceutical compositions thereof may also be used for reducing the incidence or severity of flushing and/or hot flashes which are symptoms of a disorder. In one embodiment, the compounds of the invention and pharmaceutical compositions thereof may be used to reduce flushing side effects of a vasodilator or an antilipemic agent (including anticholesteremic agents and lipotropic agents).

In another representative embodiment, the method involves the use of the compounds of the invention and pharmaceutical compositions thereof to reduce flushing side effects of antidepressants or anti-psychotic agent. For instance, the compounds of the invention and pharmaceutical compositions thereof can be used in conjunction (administered separately or together) with a serotonin reuptake inhibitor, a 5HT2 receptor antagonist, an anticonvulsant, a norepinephrine reuptake inhibitor, an alpha-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT1 A receptor antagonist, a 5HT1D receptor antagonist, a CRF antagonist, a monoamine oxidase inhibitor, or a sedative-hypnotic drug.

In certain embodiments, the compounds of the invention and pharmaceutical compositions thereof may be used as part of a treatment with a serotonin reuptake inhibitor (SRI) to reduce flushing. In certain preferred embodiments, the SRI is a selective serotonin reuptake inhibitor (SSRI), such as a fluoxetinoid (fluoxetine, norfluoxetine) or a nefazodonoid (nefazodone, hydroxynefazodone, oxonefazodone). Other exemplary SSRIs include duloxetine, venlafaxine, milnacipran, citalopram, fluvoxamine, paroxetine and sertraline. The compounds of the invention and pharmaceutical compositions thereof can also be used as part of a treatment with sedative-hypnotic drug, such as selected from the group consisting of a benzodiazepine (such as alprazolam, chlordiazepoxide, clonazepam, chlorazepate, clobazam, diazepam, halazepam, lorazepam, oxazepam and prazepam), Zolpidem, and barbiturates. In still other embodiments, the compounds of the invention and pharmaceutical compositions thereof may be used as part of a treatment with a 5-HT1 A receptor partial agonist, such as selected from the group consisting of buspirone, flesinoxan, gepirone and ipsapirone. The compounds of the invention and pharmaceutical compositions thereof can also be used as part of a treatment with a norepinephrine reuptake inhibitor, such as selected from tertiary amine tricyclics and secondary amine tricyclics. Exemplary tertiary amine tricyclics include amitriptyline, clomipramine, doxepin, imipramine and trimipramine. Exemplary secondary amine tricyclics include amoxapine, desipramine, maprotiline, nortriptyline and protriptyline. In certain embodiments, the compounds of the invention and pharmaceutical compositions thereof may be used as part of a treatment with a monoamine oxidase inhibitor, such as selected from the group consisting of isocarboxazid, phenelzine, tranylcypromine, selegiline and moclobemide.

In still another representative embodiment, the compounds of the invention and pharmaceutical compositions thereof may be used to reduce flushing side effects of chemotherapeutic agents, such as cyclophosphamide, and tamoxifen.

In another embodiment, the compounds of the invention and pharmaceutical compositions thereof may be used to reduce flushing side effects of calcium channel blockers, such as amlodipine.

In another embodiment, the compounds of the invention and pharmaceutical compositions thereof may be used to reduce flushing side effects of antibiotics. For example, the compounds of the invention and pharmaceutical compositions thereof can be used in combination with levofloxacin. Levofloxacin is used to treat infections of the sinuses, skin, lungs, ears, airways, bones, and joints caused by susceptible bacteria.

When administered together with another therapeutic agent, the disclosed compounds can be administered simultaneously in the same pharmaceutical formulation or simultaneously in separate pharmaceutical formulations. Alternatively, when administered together with another therapeutic agent, the disclosed compounds can be administered at separate times, depending the dosing requirements of the second therapeutic agent.

Pharmaceutical compositions are disclosed that include one or more compounds provided herein (such as the compound of Formulas (I) to (XII)), and typically at least one additional substance, such as an excipient, a known therapeutic other than those of the present disclosure, and combinations thereof. In some embodiments, the disclosed compounds can be used in combination with other agents known to have beneficial activity targeting diseases or disorders listed above. For example, disclosed compounds can be administered alone or in combination with one or more compounds selected from the group consisting of: PPAR S agonists, AMPK activators, PARP inhibitors, SIRT-activating compounds, nicotinamide N-methyl transferase (NNMT) inhibitors, niacin, nicotinamide, or nicotinamide riboside and its derivatives, and acetyl-CoA carboxylase inhibitors, and the pharmaceutically acceptable salts of these compounds.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The precise amount of compound administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a mitochondria-related disease using the disclosed compounds for guidance.

The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

“Pharmaceutically acceptable excipient” and “pharmaceutically acceptable carrier” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

### EXEMPLIFICATION

### Abbreviations

Ac acetyl
ACN acetonitrile
aq aqueous
Boc tert-butyloxycarbonyl
Boc-anhydride Di-tert-butyl dicarbonate
Bn benzyl
CuI copper iodide
DCM dichloromethane
DIPEA diisopropylethylamine
DMAP 4-(dimethylamino)pyridine
DMF N,N-dimethylformamide
DMSO dimethyl Sulfoxide
Dppf 1,1′-bis(diphenylphosphino)ferrocene
EDC 3-(3-dimethylaminopropyl)-1-ethylcarbodiimide
Et ethyl
EtOAc ethyl acetate
h hour
HATU 1-[Bis(dimethylamino)methylene]-1H-1,2,3-triazolo[4,5-b]pyridinium 3-oxide hexafluorophosphate
HOBt 1-hydroxybenzotriazole
HPLC high-performance liquid chromatography
K₂CO₃ Potassium carbonate
LCMS liquid chromatography-mass spectrometry
M concentration expressed in mol/L
Me methyl
MeOH methanol
Na₂SO₄ Sodium Sulfate
NaOH Sodium hydroxide
NIS N-iodosuccinimide
Ph phenyl
Prep-HPLC preparative high-performance liquid chromatography
RT room temperature
SFC supercritical fluid chromatography
SEM (trimethylsilyl)ethoxymethyl
TFA trifluoroacetic acid
THF tetrahydrofuran
Tf trifluoromethanesulfonyl
TLC thin layer chromatography
TBAF tetrabutylammonium fluoride
TMEDA N,N,N′,N′-tetramethylethylenediamine
¹H NMR (DMSO-d₆) δ (ppm) of peak in ¹H NMR in DMSO-d₆
s singlet (spectrum)
d doublet (spectrum)
t triplet (spectrum) q quartet (spectrum)
dd doublet of doublets (spectrum)
br broad peak (spectrum)
m multiplet (spectrum).
mg milligram
mM millimolar
nM nanomolar

General Information:

LCMS Analysis Condition:

Instrument name: Agilent Technologies 1290 infinity 11.

Method A: Method: A—0.1% TFA in H₂O, B—0.1% TFA in ACN; flow rate: 2.0 mL/min; column: XBridge C8 (50×4.6 mm, 3.5 μm).

Method B: Method: A—10 mM NH₄HCO₃ in H₂O, B—ACN; flow rate: 1.0 mL/min; column: XBridge C8 (50×4.6 mm, 3.5 μm).

Method C: Method: 0.1% HCOOH in H₂O:ACN (95:5); flow rate: 2.0 mL/min; column: ZORBAX XDB C-18 (50×4.6 mm, 3.5 μm).

Method D: Method: A—10 mM Ammonium acetate in H₂O, B—ACN; flow rate: 1.0 mL/min; column: XBridge C8 (50×4.6 mm, 3.5 μm).

Prep-HPLC Purification Condition:

Method A: A—0.1% TFA in H₂O, B—MeOH or ACN; column: Sunfire C18 (30×250 mm, 10 μm).

Method B: A—10 mM NH₄HCO₃ in H₂O, B—MeOH or ACN, Column: Sunfire C18 (30×250 mm, 10 μm).

### Example 1: N-((1r,4r)-4-(2-methoxyethoxy)cyclohexyl)-5-(thiazol-5-yl)-1H-indole-7-carboxamide

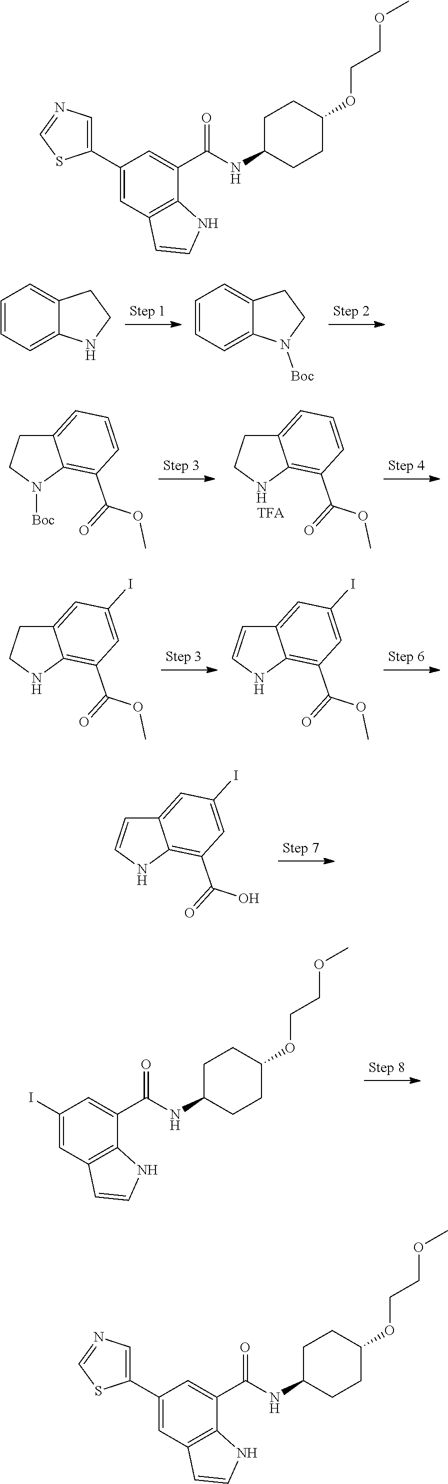

### Step 1: tert-butyl indoline-1-carboxylate

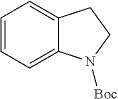 [SMILES: CC(C)(C)OC(=O)N1CCc2ccccc21]

To a stirred solution of indoline (15.0 g, 0.126 mol) in tetrahydrofuran (250 mL) Boc-anhydride (32.9 g, 0.151 mol) was added dropwise over 30 min maintaining the reaction temperature below 5° C. and then stirred at RT for overnight. Reaction was monitored by TLC and after completion of starting material the reaction mixture was quenched with water (500 mL). The resulting reaction mixture was extracted with EtOAc (3×200 mL). The combined organic layer was washed with water (200 mL), brine (200 mL) and dried over anhydrous Na₂SO₄. The solvent was evaporated in vacuum and the resulting crude product was triturated with cold petroleum ether to afford the title compound. Yield: 75% (20.5 g, white solid). ¹HNMR (400 MHz, CDCl₃): δ 7.28 (s, 1H), 7.17-7.14 (m, 2H), 6.96-6.91 (m, 1H), 3.99 (t, J=11.2 Hz, 2H), 3.10 (t, J=11.2 Hz, 2H), 1.59 (s, 9H). LCMS: (Method A) 120.3 (M−99)

### Step 2: 1-(tert-butyl) 7-methyl indoline-1, 7-dicarboxylate

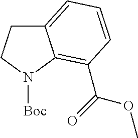 [SMILES: COC(=O)c1cccc2c1N(C(=O)OC(C)(C)C)CC2]

A solution of sec-butyl lithium (91.4 mL, 0.127 mol) was cannulated over 20 min to stirred suspension of tert-butyl indoline-1-carboxylate (20.0 g, 91.3 mmol), TMEDA (35.5 mL, 237.0 mmol) in diethyl ether (330 mL) at −75° C. Then the reaction mixture was stirred at −78° C. for 90 min and this thick suspension was added by cannula over 10 min to a stirred solution of methyl chloroformate (42.4 mL, 0.547 mol) in diethyl ether (70 mL) maintaining the reaction temperature between −78 to −50° C. Further the reaction mixture was stirred at −78° C. for 45 min and at −20° C. for 1.5 h. The reaction mixture was monitored by TLC, the TLC shown 30% starting material was unreacted. The reaction mixture was quenched by dropwise addition of methanol (100 mL) and stirred for 30 min at 0° C. followed by Sat. NH₄Cl solution (50 mL) stirred for 30 min at RT. Two layers were separated; aqueous phase was extracted with EtOAc (3×50 mL). The combined organic layer was washed with water (200 mL), brine (200 mL), dried over anhydrous Na₂SO₄ and the solvent was evaporated in vacuum. The resulting crude product was purified by column chromatography (100-200 mesh silica gel eluting with 0-10% EtOAc in petroleum ether) as gradient to afford the title compound Yield: 52% (13.0 g, white solid). ¹HNMR (400 MHz, CDCl₃): δ 7.52 (d, J=7.8 Hz, 1H), 7.30 (d, J=7.48 Hz, 1H), 7.03 (t, J=7.6 Hz, 1H), 4.13 (t, J=8.2 Hz, 2H), 3.85 (s, 3H), 3.09 (t, J=8.2 Hz, 2H), 1.58 (s, 9H). LCMS: (Method A) 178.1 (M−99)

### Step 3: Methyl 1-(2, 2, 2-trifluoroacetyl)-1l4-indoline-7-carboxylate

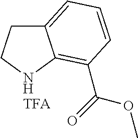

To a stirred solution of 1-(tert-butyl) 7-methyl indoline-1,7-dicarboxylate (13.0 g, 46.9 mmol) in DCM (30 mL) TFA (27.6 mL, 361.3 mmol) was added dropwise over 10 min at 0° C. and stirred for 4 h at RT. The reaction mixture was monitored by TLC. After completion the reaction mixture was concentrated under vacuum and the resulting crude was triturated with acetonitrile (2×50 mL) and diethyl ether to afford the title compound Yield: 82% (10.5 g, Brown solid). ¹H NMR (400 MHz, CDCl₃): δ 7.76-7.73 (m, 3H), 7.40-7.38 (m, 1H), 7.02 (t, J=7.6 Hz, 1H), 3.91 (s, 3H), 3.90 (t, J=8.4 Hz, 2H), 3.22 (t, J=8.4 Hz, 2H). LCMS: (Method A) 178.1 (M+H)

### Step 4: Methyl 5-iodoindoline-7-carboxylate

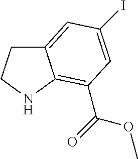

To a stirred solution of methyl 1-(2,2,2-trifluoroacetyl)-1l4-indoline-7-carboxylate (10.5 g, 382.3 mmol) in acetonitrile (150 mL) NIS (9.4 g, 421.2 mmol) was added portion wise over 10 min at −10° C. and stirred at same temperature for 40 min. The reaction mixture was monitored by TLC and quenched with 10% Na₂S203 (50 mL). The resulting suspension was stirred for 15 min at 0° C. and solids were collected by filtrations to afford the title compound. Yield: 83% (9.6 g, Yellow solid). ¹HNMR (400 MHz, CDCl₃): δ 7.63 (d, J=1.2 Hz, 1H), 7.38 (s, 1H), 6.72 (s, 1H), 3.76 (s, 3H), 3.59 (t, J=8.8 Hz, 2H), 2.97 (t, J=8.8 Hz, 2H). LCMS: (Method A) 304.1 (M+H)

### Step 5: Methyl 5-iodo-1H-indole-7-carboxylate

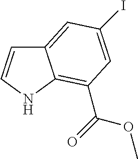

To a stirred solution of Methyl 5-iodoindoline-7-carboxylate (9.6 g, 0.0316 mol) in toluene (180 mL) MnO₂ (13.76 g, 0.158 mol) was added portion wise over 20 min at RT and heated at 75° C. for 2 h. The reaction mixture was filtered through Celite bed and washed with DCM (2×100 mL). The filtrate was evaporated under vacuum and resulting crude compound was purified by column chromatography (100-200 mesh silica gel, 5% EtOAc in petroleum ether) to afford the title compound. Yield: 81% (7.7 g, yellow solid). ¹HNMR (400 MHz, DMSO-d₆): δ 11.34 (br s, 1H), 8.23 (d, J=1.1 Hz, 1H), 7.96 (d, J=1.5 Hz, 1H), 7.43 (t, J=2.8 Hz, 1H), 6.55-6.54 (m, 1H), 3.94 (s, 3H). LCMS: (Method A) 300.0 (M−H)

### Step 6: 5-Iodo-1H-indole-7-carboxylic acid

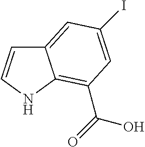 [SMILES: O=C(O)c1cc(I)cc2cc[nH]c12]

To a stirred solution of Methyl 5-iodo-1H-indole-7-carboxylate (2.0 g, 6.4 mmol) in MeOH (16 mL) and THF (16 mL), 3M NaOH (16.0 mL, 47.8 mmol) solution was added at RT and stirred at same temperature for 1.5 h. The reaction mixture was monitored by TLC, starting material was consumed. The reaction mixture was evaporated under vacuum and sodium salt was acidified with 3 N HCl solution (20 mL). The resulting solid was collected by filtration to afford the title compound. Yield: 95% (1.8 g, White solid). ¹HNMR 400 MHz, DMSO-d₆): δ 13.34 (br s, 1H), 11.24 (s, 1H), 8.18 (d, J=1.2 Hz, 1H), 7.93 (d, J=1.6 Hz, 1H), 7.38-7.36 (m, 1H), 6.52-6.51 (m, 1H). LCMS: (Method B) 286.0 (M−H)

### Step 7: 5-iodo-N-((1r,4r)-4-(2-methoxyethoxy) cyclohexyl)-1H-indole-7-carboxamide

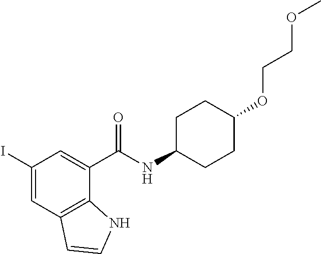 [SMILES: COCCO[C@H]1CC[C@H](NC(=O)c2cc(I)cc3cc[nH]c23)CC1]

To a stirred solution of 5-Iodo-1H-indole-7-carboxylic acid (1.78 g, 6.2 mmol) in DMF (10 mL) were added DIPEA (1.4 mL, 8.0 mmol) and HATU (2.80 g, 7.4 mmol) at 0° C. After stirring for 5 min a solution of (1r,4r)-4-(2-methoxyethoxy)cyclohexan-1-amine (1.27 g, 7.4 mmol) in DMF (2.5 mL) was added at same temperature. Further reaction was stirred at RT for 2.5 h. The reaction mixture was monitored by TLC and quenched with water (25 mL). The resulting suspension was extracted with EtOAc (3×25 mL). The combined organic layer was washed with water (20 mL), brine (20 mL), dried over anhydrous Na₂SO₄ and solvent was evaporated under vacuum. The resulting crude product was purified by column chromatography (100-200 mesh silica gel, 10-50% EtOAc in petroleum ether) to afford the title compound Yield: 59% (1.62 g, White solid). Further 100 mg taken for trituration with ether to get more purity to submit for assay. ¹HNMR (400 MHz, DMSO-d₆): δ 11.25 (s, 1H), 8.41 (d, J=7.6 Hz, 1H), 8.07 (s, 1H), 7.92 (s, 1H), 7.33 (s, 1H), 6.44 (s, 1H), 3.87-3.82 (m, 1H), 3.56-3.53 (m, 2H), 3.44-3.42 (m, 2H), 3.22-3.23 (m, 4H), 2.05-2.02 (m, 2H), 1.99-1.88 (m, 2H), 1.46-1.37 (m, 2H), 1.28-1.23 (m, 2H). LCMS: (Method A) 443.0 (M+H)

### Step 8: N-((1r,4r)-4-(2-methoxyethoxy)cyclohexyl)-5-(thiazol-5-yl)-1H-indole-7-carboxamide

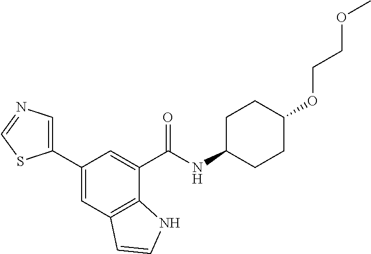 [SMILES: COCCO[C@H]1CC[C@H](NC(=O)c2cc(-c3cncs3)cc3cc[nH]c23)CC1]

To a stirred solution of 5-iodo-N-((1r,4r)-4-(2-methoxyethoxy)cyclohexyl)-1H-indole-7-carboxamide (200 mg, 0.452 mmol) in 5 mL ethanol and water (0.01 mL), 5-(4,4,5,5-tetramethyl-1,3,2-dioxaborolan-2-yl)thiazole (114 mg, 0.542 mmol), CuI (4.29 mg, 0.0226 mmol), potassium carbonate (187 mg, 1.35 mmol) were added at RT and the nitrogen gas was purged through the reaction mixture for 2 min before addition of Pd(dppf)Cl₂.DCM (9.5 mg, 0.0135 mmol). Then the reaction mixture was heated at 90° C. for 15 h. After completion of the reaction, the reaction mixture was filtered through Celite, washed with DCM (10 mL) and filtrate was evaporated under vacuum. The resulting crude was purified by Prep-HPLC (method A) to get off-white solid product. Yield: 18% (33.98 mg, off-white solid). ¹HNMR 400 MHz, DMSO-d6): δ 11.25 (s, 1H), 9.05 (s, 1H), 8.45 (d, J=7.6 Hz, 1H), 8.29 (s, 1H), 8.00 (s, 1H), 8.0 (s, 1H), 7.4 (t, J=2.8 Hz, 1H), 6.5 (d, J=2.0 Hz, 1H), 3.9 (q, J=3.6 Hz, 1H), 3.57-3.56 (m, 2H), 3.45-3.44 (m, 2H), 3.29-3.28 (m, 4H), 2.07-2.05 (m, 2H), 1.96-1.93 (m, 2H), 1.49-1.43 (m, 2H), 1.28-1.23 (m, 2H). LCMS: (Method C) 400.3 (M+H)

### Example 2: N-((1r,4r)-4-(2-methoxyethoxy)cyclohexyl)-5-(1H-pyrazol-1-yl)-1H-indole-7-carboxamide

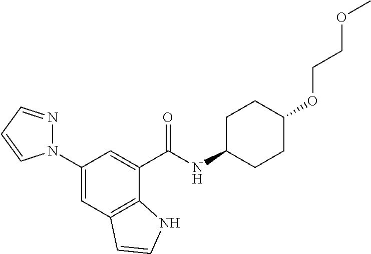 [SMILES: COCCO[C@H]1CC[C@H](NC(=O)c2cc(-n3cccn3)cc3cc[nH]c23)CC1]

To a stirred solution of 5-iodo-N-((1r,4r)-4-(2-methoxyethoxy)cyclohexyl)-1H-indole-7-carboxamide (100 mg, 0.226 mmol) in DMF (5 mL), K₂CO₃ (94 mg, 0.679 mmol) and 1H-imidazole (46 mg, 0.679 mmol) were added. Then N2 gas was purged for 5 minutes before addition of Cu(I)I (4.5 mg, 0.023 mmol) into sealed tube and stirring was continued at 130° C. for 16 h. Reaction was monitoring by TLC, starting was consumed. The reaction mixture was filtered through the Celite bed and filtrate was evaporated in vacuum. The resulting residue was purified by Prep-HPLC (Method B) to get titled product as a white solid product. Yield: 29% (25 mg, white solid) ¹H NMR (400 MHz, DMSO-d₆): δ 11.23 (s, 1H), 8.49 (d, J=7.96 Hz, 1H), 8.43 (d, J=1.8 Hz, 1H), 8.09-8.07 (m, 2H), 7.73 (d, J=1.4 Hz, 1H), 7.42 (s, 1H), 6.55 (t, J=1.7 Hz, 1H), 3.88-3.86 (br, s, 1H), 3.55 (dd, J=4.2, 2.2 Hz, 2H), 3.43 (dd, J=3.56, 1.32 Hz, 2H), 3.25-3.24 (m, 4H), 2.07-2.05 (m, 2H), 1.95-1.92 (m, 2H), 1.49-1.40 (m, 2H), 1.31-1.22 (m, 2H). LCMS: (Method A) 383.1 (M+H)

### Example 3: N-((1r,4r)-4-(2-methoxyethoxy)cyclohexyl)-5-(1H-1,2,4-triazol-1-yl)-1H-indole-7-carboxamide

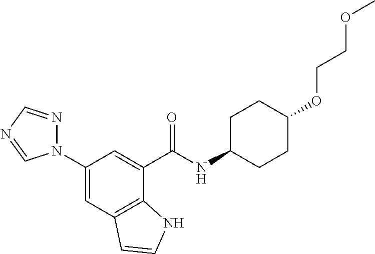 [SMILES: COCCO[C@H]1CC[C@H](NC(=O)c2cc(-n3cncn3)cc3cc[nH]c23)CC1]

To a reaction mixture of 5-iodo-N-((1r,4r)-4-(2-methoxyethoxy)cyclohexyl)-1H-indole-7-carboxamide (200 mg, 0.452 mmol), 4H-1,2,4-triazole (46.8 mg, 0.678 mmol, copper iodide (8.59 mg, 0.0452 mmol), L-proline (46.8 mg, 0.407 mmol) and K₂CO₃ (124.8 mg, 0.904 mmol) in DMF (5 mL) TMEDA (105.1 mg, 0.904 mmol) was added and purged with N2 for 5 min. Then the reaction mixture was heated in a sealed tube at 120° C. for 16 h. Reaction was monitored by LCMS. The reaction mixture was diluted with DCM and filtered through Celite bed, washed with DCM. The filtrate was evaporated in vacuum to get crude compound which was purified by flash chromatography Biotage Isolera (230-400 mesh silica gel, 0-10% MeOH in DCM) to get titled compound. Yield: 22% (32.34 mg, off white solid). ¹HNMR (400 MHz, DMSO-d₆): δ 11.26 (s, 1H), 9.16 (s, 1H), 8.47 (d, J=7.6 Hz, 1H), 8.23 (s, 1H), 8.11 (d, J=1.6 Hz, 1H), 8.06 (d, J=1.6 Hz, 1H), 7.46 (d, J=3.2 Hz, 1H), 6.59 (d, J=2.8 Hz, 1H), 3.88-3.85 (m, 1H), 3.55-3.53 (m, 2H), 3.43-3.41 (m, 2H), 3.27-3.25 (m, 1H), 3.24 (s, 3H), 2.05-2.03 (m, 2H), 1.94-1.91 (m, 2H), 1.44-1.38 (m, 2H), 1.30-1.24 (m, 2H). LCMS: (Method C) 384.1 (M+H)

### Example 4: N-((1r,4r)-4-(2-methoxyethoxy)cyclohexyl)-5-(5H-tetrazol-5-yl)-1H-indole-7-carboxamide

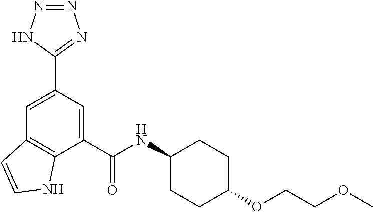 [SMILES: COCCO[C@H]1CC[C@H](NC(=O)c2cc(-c3nnn[nH]3)cc3cc[nH]c23)CC1]

To a stirred suspension of 5-cyano-N-((1r,4r)-4-(2-methoxyethoxy)cyclohexyl)-1H-indole-7-carboxamide (0.1 g, 0.29 mmol), LiCl (18.6 mg, 0.43 mmol) and NH₄Cl (78 mg, 1.46 mmol) in DMF (3 mL) at RT was added NaN₃ (95 mg, 1.46 mmol) and the reaction mixture was heated to 125° C. for 48 h. After completion, the reaction mixture was cooled to RT and filtered through Celite. The Celite bed was washed with 10% MeOH in DCM (20 mL) and the combined filtrate was concentrated under reduced pressure. The crude residue was purified by flash chromatography on Biotage Isolera (silica gel: 230-400 mesh, eluent: 4% MeOH in DCM) followed by prep. HPLC (Method B) to get the title compound. Yield: 5% (6.06 mg, off white solid). ¹H NMR (400 MHz, DMSO-d₆): δ 11.22 (s, 1H), 8.56 (d, J=8.0 Hz, 1H), 8.32 (d, J=4.0 Hz, 2H), 8.15 (s, 1H) 7.39 (s, 1H), 6.58 (s, 1H), 3.91-3.84 (m, 1H), 3.56 (br s, 2H), 3.45-3.42 (m, 2H), 3.40-3.34 (m, 1H), 3.30-3.22 (m, 4H), 2.05 (d, J=10.4 Hz, 2H), 1.92 (d, J=11.2 Hz, 2H), 1.53-1.44 (m, 2H), 1.31-1.23 (m, 2H). LCMS: (Method C) 385.1 (M+H)

### Example 5: N-((1r,4r)-4-(2-methoxyethoxy)cyclohexyl)-5-(thiazol-4-yl)-1H-indole-7-carboxamide

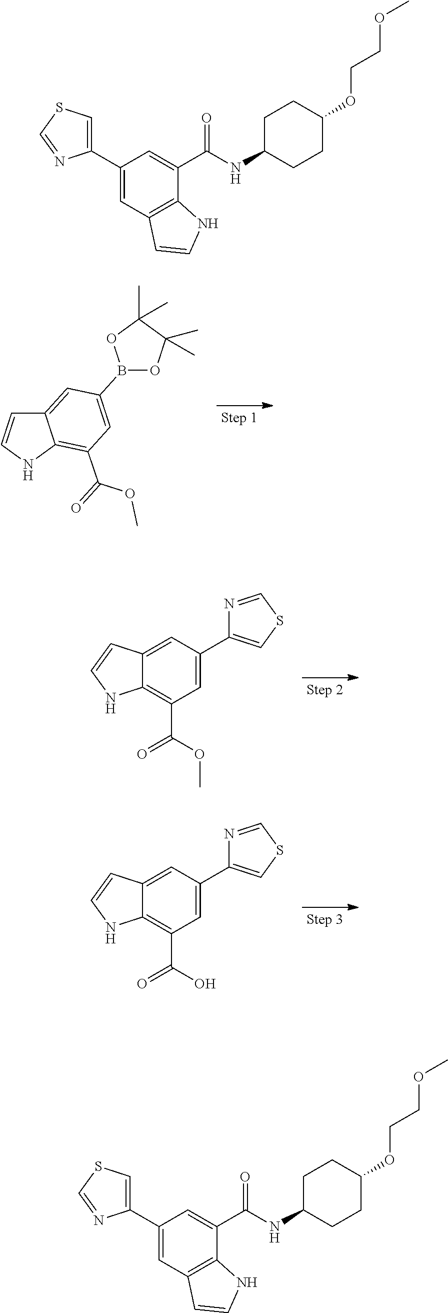

### Step 1: methyl 5-(thiazol-4-yl)-1H-indole-7-carboxylate

To a stirred solution of methyl 5-(4,4,5,5-tetramethyl-1,3,2-dioxaborolan-2-yl)-1H-indole-7-carboxylate (150 mg, 0.498 mmol) in dioxane (3.0 mL) water, 4-bromothiazole (80 mg, 0.498 mmol) and K₂CO₃ (137.5 mg, 0.996 mmol) were added at RT and the nitrogen gas was purged through the reaction mixture for 5 min before addition of PdCl₂(dppf).DCM (40.6 mg, 0.0498 mmol). Then the reaction mixture was heated in seal tube at 100° C. for 5 h. Reaction was monitored by TLC, the reaction mixture was filtered through Celite bed and washed with DCM (20 mL). The filtrate was evaporated under vacuum to get crude product which was purified by flash chromatography (Biotage Isolera, 100-200 mesh silica gel, 0-50% EtOAc in petroleum ether) to afford the title compound Yield: 78% (100 mg, off white solid). ¹HNMR (400 MHz, DMSO-d6: δ 11.27 (s, 1H), 9.20 (s, 1H), 8.49 (s, 1H), 8.46 (s, 1H), 8.14 (s, 1H), 7.46 (s, 1H), 6.64 (s, 1H), 3.98 (s, 3H). LCMS: (Method C) 259.0 (M+H)

### Step 2: 5-(thiazol-4-yl)-1H-indole-7-carboxylic acid

To a stirred solution of methyl 5-(thiazol-4-yl)-1H-indole-7-carboxylate (100 mg, 0.387 mmol) in MeOH (2 mL) and THF (2 mL), 3M NaOH (0.9 mL, 2.71 mmol) solution was added at RT and stirred at same temperature for 3 h. The reaction mixture was monitored by TLC, starting material was consumed. The reaction mixture was evaporated under vacuum and acidified with 1.5 N HCl solution (3 mL). The resulting solid was collected by filtrations to afford the title compound. Yield: 89% (80 mg, off white solid). ¹HNMR (400 MHz, DMSO-d₆): δ 11.12 (br s, 1H), 9.17 (d, J=1 88 Hz, 1H), 8.31-8.30 (m, 2H), 7.99 (d, J=1.9 Hz, 1H), 7.36-7.32 (m, 1H), 6.52 (s, 1H). LCMS: (Method C) 245.1 (M+H)

### Step 3: N-((1r,4r)-4-(2-methoxyethoxy)cyclohexyl)-5-(thiazol-4-yl)-1H-indole-7-carboxamide

To a stirred solution of 5-(thiazol-4-yl)-1H-indole-7-carboxylic acid (80 mg, 0.327 mmol) in DMF (3 mL) were added DIPEA (54.9 mg, 0.426 mmol) and HATU (149.5 mg, 0.393 mmol) at 0° C. After stirring for 5 min a solution of (1r, 4r)-4-(2-methoxyethoxy) cyclohexan-1-amine (62.3 mg, 0.360 mmol) in DMF (0.5 mL) followed by DIPEA (54.9 mg, 0.42 mmol) were added at same temperature. Further reaction was stirred at RT for 16 h. The reaction mixture was monitored by TLC and quenched with water (2.5 mL). The resulting suspension was extracted with DCM (3×10 mL). The combined organic layer was washed with water (20 mL), brine (20 mL), dried over anhydrous Na₂SO₄ and solvent was evaporated under vacuum to get the crude product which was purified by flash chromatography (Biotage Isolera, 230-400 mesh silica gel eluting with 0-10% MeOH in DCM) to afford the title compound. Further it was purified by Prep-HPLC (Method A). The collected prep. HPLC fraction was concentered under vacuum. The resulting residue was dissolved in DCM and neutralized with 10% NaHCO₃ aqueous solution. The organic phase was washed with water, brine, dried over anhydrous Na₂SO₄ and evaporated to afford the title compound. Yield: 18% (23.94 mg, off white solid). ¹HNMR (400 MHz, DMSO-d₆): δ 11.14 (s, 1H), 9.20 (d, J=2.0 Hz, 1H), 8.42 (d, J=8.0 Hz, 1H), 8.33 (d, J=0.8 Hz, 1H), 8.23 (d, J=1.6 Hz, 1H), 8.03 (d, J=2.0 Hz, 1H), 7.36 (t, J=2.8 Hz, 1H), 6.54-6.53 (m, 1H), 3.91-3.84 (m, 1H), 3.56-3.53 (m, 2H), 3.44-3.41 (m, 2H), 3.24-3.25 (m, 4H), 2.06-2.04 (m, 2H), 1.95-1.92 (m, 2H), 1.51-1.41 (m, 2H), 1.31-1.24 (m, 2H). LCMS: (Method C) 400.0 (M+H)

### Example 6: 5-(1H-imidazol-2-yl)-N-((1r,4r)-4-(2-methoxyethoxy)cyclohexyl)-1H-indole-7-carboxamide

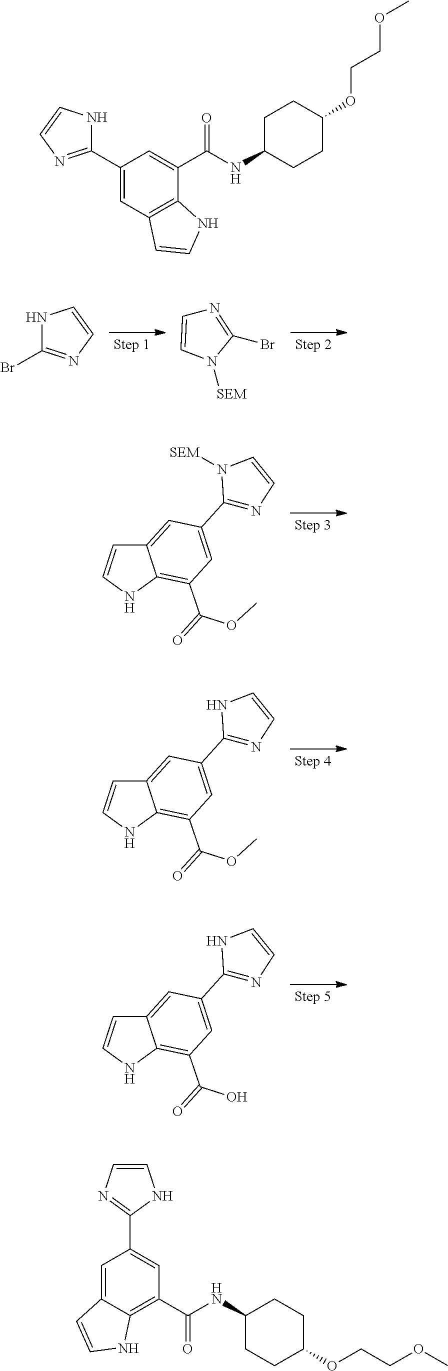

### Step 1: 2-bromo-1-((2-(trimethylsilyl) ethoxy) methyl)-1H-imidazole

To a stirred solution of 2-bromo-1H-imidazole (1.5 g, 10.2 mmol) in DMF (10.0 mL) was added NaH (0.489 g, 12.4 mmol, 60% suspension) portion wised at 0° C. and stirred at same temperature for 10 min before addition of SEM-Cl (2.03 g, 12.4 mmol). Then the reaction mixture was stirred at RT for 16 h. and quenched with ice cold water. The resulting suspension was extracted with EtOAc (3×50 mL). The combined organic layer was washed with water (25 mL), brine (25 mL), dried over anhydrous Na₂SO₄ and solvent was evaporated in vacuum to get crude compound which was purified by flash chromatography (Biotage Isolera, 100-200 mesh silica gel, 0-40% EtOAc in petroleum ether) to afford the title compound. Yield: 63% (1.75 g, colorless oil). ¹HNMR (400 MHz, DMSO-d6): δ 7.48 (d, J=2.0 Hz, 1H), 6.96 (d, J=2.0 Hz, 1H), 5.28 (s, 2H). 3.51 (t, J=10.8 Hz, 2H), 0.84 (t, J=10.8 Hz, 2H), 0.04 (s, 9H). LCMS: (Method C) 279.1 (M+1)

### Step 2: Methyl 5-(1-((2-(trimethylsilyl)ethoxy)methyl)-1H-imidazol-2-yl)-1H-indole-7-carboxylate

To a stirred solution of methyl 5-(4,4,5,5-tetramethyl-1,3,2-dioxaborolan-2-yl)-1H-indole-7-carboxylate (250 mg, 0.083 mmol) in dioxane (4.5 mL) and water (0.5 mL), 2-bromo-1-((2-(trimethylsilyl)ethoxy)methyl)-1H-imidazole (299 mg, 1.07 mmol), K₂CO₃ (230 mg, 1.66 mmol) were added at RT and the nitrogen gas was purged through the reaction mixture for 5 min before addition of PdCl₂(dppf).DCM (33 mg, 0.041 mmol) and catcaxium (29 mg, 0.083 mmol). Then the reaction mixture was heated in seal tube at 110° C. for 16 h. Reaction was monitored by TLC, after completion of the reaction, the reaction mixture was filtered through Celite bed and washed with DCM (20 mL). The filtrate was evaporated under vacuum. The resulting crude product which was purified by flash chromatography (Biotage Isolera, 100-200 mesh silica gel, 0-80% EtOAc in Petroleum ether) to afford the title compound. Yield: 75% (230 mg, colorless oil). ¹HNMR (400 MHz, DMSO-d₆): δ 11.36 (s, 1H), 8.26 (s, 1H), 8.23 (s, 1H), 7.49 (d, J=2.4 Hz, 1H), 7.45 (d, J=0.8 Hz, 1H), 7.02 (s, 1H), 6.64 (s, 1H), 5.35 (s, 2H). 3.94 (s, 3H). 3.61 (t, J=8.4 Hz, 2H), 0.90 (t, J=8.8 Hz, 2H), 0.03 (s, 9H). LCMS: (Method C) 372.2 (M+H)

### Step 3: Methyl 5-(1H-imidazol-2-yl)-1H-indole-7-carboxylate

To a stirred solution of methyl 5-(1-((2-(trimethylsilyl)ethoxy)methyl)-1H-imidazol-2-yl)-1H-indole-7-carboxylate (400 mg, 1.07 mmol) in DCM (10 mL) was added TFA (61 mg, 5.38 mmol) at RT and stirred at same temperature for 16 h. The reaction mixture was evaporated under vacuum and resulting solid was triturated with ether (10 mL) to afford the title compound. Yield: 71% (80 mg, off white solid). LCMS: (Method C) 242.1 (M+H)

### Step 4: 5-(1H-imidazol-2-yl)-1H-indole-7-carboxylic acid

To a stirred solution of methyl 5-(1H-imidazol-2-yl)-1H-indole-7-carboxylate (120 mg, 0.497 mmol) in MeOH (3 mL) and THF (3 mL) was added NaOH (60 mg, 1.49 mmol) and stirred at RT for 6 h. The reaction mixture was evaporated under vacuum and acidified with 1.5 N HCl solution (3 mL). The resulting solid was collected by filtrations to afford the title compound. Yield: 71% (80 mg, off white solid). LCMS: (Method C) 228.1 (M+H)

### Step 5: 5-(1H-imidazol-2-yl)-N-((1r,4r)-4-(2-methoxyethoxy)cyclohexyl)-1H-indole-7-carboxamide

To a stirred solution of 5-(1H-imidazol-2-yl)-1H-indole-7-carboxylic acid (80 mg, 0.352 mmol) in DMF (4 mL) were added DIPEA (59.1 mg, 0.458 mmol) and HATU (160.7 mg, 0.422 mmol) at 0° C. After stirring for 5 min a solution of (1r, 4r)-4-(2-methoxyethoxy) cyclohexan-1-amine (73.1 mg, 0.422 mmol) in DMF (1.0 mL) followed by was DIPEA (59.1 mg, 0.45 mmol) were added at same temperature. Further reaction was stirred at RT for 16 h.

The reaction mixture was monitored by TLC and quenched with water (2.5 mL). The resulting suspension was extracted with DCM (3×10 mL). The combined organic layer was washed with water (10 mL), brine (10 mL), dried over anhydrous Na₂SO₄ and solvent was evaporated under vacuum. The resulting crude product which was purified by flash chromatography (Biotage Isolera, 230-400 mesh silica gel, 0-10% MeOH in DCM) to afford the title compound. Yield: 37% (50.04 mg, off white solid). ¹HNMR (400 MHz, DMSO-d₆): δ 12.67 (s, 1H), 11.20 (s, 1H), 8.43 (d, J=8.0 Hz, 1H), 8.21 (s, 2H), 7.38 (s, 1H), 7.18 (s, 2H), 6.55 (m, 1H), 3.89-3.86 (m, 1H), 3.57-3.54 (m, 2H), 3.45-3.42 (m, 2H), 3.26-3.24 (m, 4H), 2.07-2.04 (m, 2H), 1.94-1.91 (m, 2H), 1.51-1.42 (m, 2H), 1.31-1.23 (m, 2H). LCMS: (Method C) 383.0 (M+H)

### Example 7: N-((1r,4r)-4-(2-methoxyethoxy)cyclohexyl)-5-(1H-pyrazol-4-yl)-1H-indole-7-carboxamide

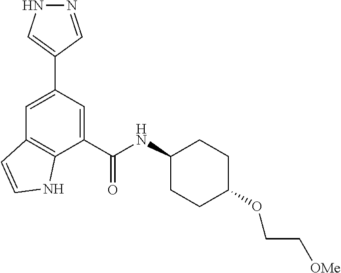 [SMILES: COCCO[C@H]1CC[C@H](NC(=O)c2cc(-c3cn[nH]c3)cc3cc[nH]c23)CC1]

To a stirred solution of 5-iodo-N-((1r,4r)-4-(2-methoxyethoxy)cyclohexyl)-1H-indole-7-carboxamide (150 mg, 0.33 mmol) in a mixture of 1,2-dimethoxyethane (5 mL) and water (0.5 mL), tert-butyl 4-(4,4,5,5-tetramethyl-1,3,2-dioxaborolan-2-yl)-1H-pyrazole-1-carboxylate (149 mg, 0.50 mmol) and K₂CO₃ (117 mg, 0.84 mmol) were added at RT. The reaction mixture was purged with nitrogen gas 10 min at RT. Then Pd(dppf)Cl₂.DCM (27.6 mg, 0.03 mmol) was added and the reaction mixture was heated at 100° C. for 16 h. After completion, the reaction mixture was filtered through Celite, and the filtration bed was washed with EtOAc (200 mL). The combined filtrate was dried over anhydrous Na₂SO₄, filtered and the filtrate was concentrated under vacuum. The resulting crude residue was purified by flash chromatography on Biotage Isolera (silica gel: 100-200 mesh, 2% to 3% MeOH in DCM) followed by Prep-HPLC (Method A). The prep-fraction was concentrated under reduced pressure, the residue was diluted with 10% MeOH in DCM (10 mL) and washed with brine (5 mL) and water (10 mL). The organic layer was dried over anhydrous Na₂SO₄, filtered, concentrated under reduced pressure and lyophilized to get the title compound (Boc group was removed under this condition). Yield: 42% (55.3 mg, pale brown solid). ¹H NMR (400 MHz, DMSO-d₆): 12.85 (s, 1H), 11.03 (s, 1H), 8.29 (d, J=8.0 Hz, 1H), 8.15 (s, 1H), 7.96-7.88 (m, 3H), 7.31 (d, J=2.4 Hz, 1H), 6.44 (s, 1H), 3.92-3.84 (m, 1H), 3.57-3.54 (m, 2H), 3.45-3.42 (m, 2H), 3.31-3.26 (m, 4H), 2.08-2.05 (m, 2H), 1.96-1.93 (m, 2H), 1.50-1.41 (m, 2H), 1.31-1.23 (m, 2H), LCMS: (Method C) 383.3 (M+H)

### Example 8: 3-(1-hydroxyethyl)-5-(1H-imidazol-1-yl)-N-((1r,4r)-4-(2-methoxyethoxy)cyclohexyl)-1H-indole-7-carboxamide

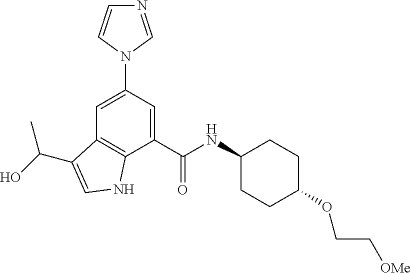 [SMILES: COCCO[C@H]1CC[C@H](NC(=O)c2cc(-n3ccnc3)cc3c(C(C)O)c[nH]c23)CC1]

To a stirred solution of 3-formyl-5-(1H-imidazol-1-yl)-N-((1r,4r)-4-(2-methoxyethoxy)cyclohexyl)-1H-indole-7-carboxamide (0.1 g, 0.24 mmol) in THF (5 mL) at 0° C., methyl magnesium bromide (0.975 mL, 2.0 M in THF, 1.95 mmol) was added slowly and the reaction mixture was stirred at RT for 3 h. After completion (monitored by TLC), the reaction mixture was quenched with aq. HCl (1.5 N) and extracted with 10% MeOH in DCM (2×10 mL). The combined organic layer was concentrated under reduced pressure and the resulting crude residue was purified by Prep-HPLC (Method A). The prep fraction was concentrated under reduced pressure. The residue was diluted with 10% MeOH in DCM (10 mL) and neutralized with sat. aq. NaHCO₃ solution. The organic layer was dried over anhydrous Na₂SO₄, filtered, concentrated under reduced pressure and lyophilized to get the title compound. Yield: 20% (21.36 mg, off white solid). ¹H NMR (400 MHz, DMSO-d₆): δ 11.05 (s, 1H), 8.38 (br s, 1H), 8.16 (s, 1H), 7.96 (s, 1H), 7.84 (s, 1H), 7.70 (s, 1H), 7.29 (s, 1H), 7.13 (s, 1H), 5.10-4.89 (m, 2H), 3.90-3.83 (m, 1H), 3.57-3.54 (m, 2H), 3.44-3.40 (m, 2H), 3.30-3.25 (m, 4H), 2.07-2.04 (m, 2H), 1.95-1.92 (m, 2H), 1.50 (d, J=6.4 Hz, 3H), 1.46-1.25 (m, 4H). LCMS: (Method C) 427.0 (M+H)

### Example 9 and Example 10: 3-((S)-1-hydroxyethyl)-5-(1H-imidazol-1-yl)-N-((1r,4r)-4-(2-methoxyethoxy)cyclohexyl)-1H-indole-7-carboxamide and 3-((R)-1-hydroxyethyl)-5-(1H-imidazol-1-yl)-N-((1r,4r)-4-(2-methoxyethoxy)cyclohexyl)-1H-indole-7-carboxamide

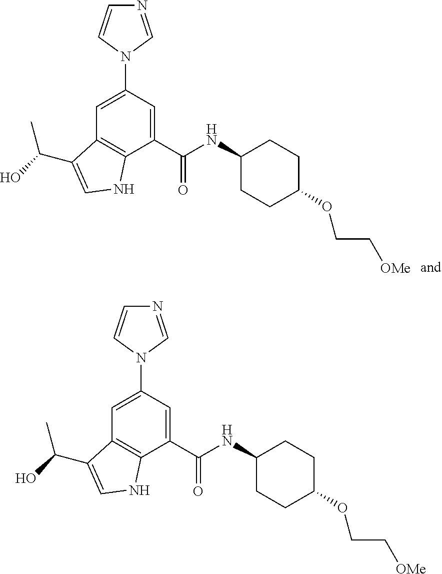 [SMILES: COCCO[C@H]1CC[C@H](NC(=O)c2cc(-n3ccnc3)cc3c([C@@H](C)O)c[nH]c23)CC1 ; COCCO[C@H]1CC[C@H](NC(=O)c2cc(-n3ccnc3)cc3c([C@H](C)O)c[nH]c23)CC1]

The enantiomers of the racemic compound Example 8 (210 mg) were separated by chiral SFC. Peak-1 was concentrated and lyophilized to get the title compound. Yield: 19% (40.85 mg, white solid). ¹H NMR (400 MHz, DMSO-d₆): δ 11.04 (s, 1H), 8.37 (d, J=7.6 Hz, 1H), 8.15 (s, 1H), 7.96 (d, J=1.6 Hz, 1H), 7.84 (d, J=2.0 Hz, 1H), 7.69 (s, 1H), 7.28 (d, J=0.8 Hz, 1H), 7.12 (s, 1H), 5.08-5.03 (m, 1H), 4.96 (d, J=4.8 Hz, 1H), 3.89-3.81 (m, 1H), 3.55-3.52 (m, 2H), 3.43-3.41 (m, 2H), 3.30-3.24 (m, 4H), 2.06-2.03 (m, 2H), 1.94-1.91 (m, 2H), 1.49 (d, J=6.0 Hz, 3H), 1.45-1.36 (m, 2H), 1.29-1.23 (m, 2H). LCMS: (Method A) 427.2 (M+H) Chiral SFC: (Mobile Phase: 0.5% isopropylamine in methanol, flow rate: 3 mL/min; column: YMC Cellulose SC (250×4.6 mm, 5 m)) 6.15 min, 98.73% ee (First Eluting).

Yield: 18% (39.49 mg, white solid). ¹H NMR (400 MHz, DMSO-d₆): δ 11.04 (s, 1H), 8.37 (d, J=7.6 Hz, 1H), 8.15 (s, 1H), 7.96 (d, J=1.2 Hz, 1H), 7.84 (d, J=1.6 Hz, 1H), 7.69 (s, 1H), 7.28 (d, J=2.4 Hz, 1H), 7.12 (s, 1H), 5.08-5.03 (m, 1H), 4.96 (d, J=4.8 Hz, 1H), 3.89-3.81 (m, 1H), 3.55-3.52 (m, 2H), 3.43-3.41 (m, 2H), 3.30-3.24 (m, 4H), 2.06-2.03 (m, 2H), 1.94-1.91 (m, 2H), 1.49 (d, J=6.4 Hz, 3H), 1.45-1.36 (m, 2H), 1.29-1.23 (m, 2H). LCMS: (Method A) 427.2 (M+H) Chiral SFC: (Mobile Phase: 0.5% isopropylamine in methanol, flow rate: 3 mL/min; column: YMC Cellulose SC (250×4.6 mm, 5 m)) 6.63 min, 93.57% ee Second Eluting).

### Example 11: 5-(1H-imidazol-1-yl)-7-(((1r,4r)-4-(2-methoxyethoxy)cyclohexyl)carbamoyl)-1H-indole-3-carboxylic acid

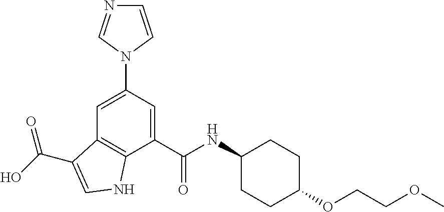 [SMILES: COCCO[C@H]1CC[C@H](NC(=O)c2cc(-n3ccnc3)cc3c(C(=O)O)c[nH]c23)CC1]

To a stirred solution of 3-formyl-5-(1H-imidazol-1-yl)-N-((1r,4r)-4-(2-methoxyethoxy) cyclohexyl)-1H-indole-7-carboxamide (0.10 g, 0.24 mmol) and KOH (41 mg, 0.73 mmol) in a mixture of acetone (3 mL) and water (0.5 mL) at RT, was added KMnO4 (77 mg, 0.49 mmol) slowly and the reaction mixture was stirred at RT for 16 h. The reaction mixture was monitored by TLC. After completion, the reaction mixture was filtered through a Celite pad and the filtrate was acidified with aq. HCl (1.5 N). The filtrate was concentrated under reduced pressure and the resulting crude residue was purified by Prep-HPLC (Method A) to get the title compound. Yield: 5% (4.88 mg, off white solid). ¹HNMR (400 MHz, DMSO-d₆) δ 11.90 (s, 1H), 8.52 (d, J=6.8 Hz, 1H), 8.22 (d, J=2.0 Hz, 1H), 8.18 (s, 1H), 7.98 (s, 1H), 7.97 (d, J=2.0 Hz, 1H), 7.72 (s, 1H), 7.14 (s, 1H), 3.89-3.82 (m, 1H), 3.57-3.54 (m, 2H), 3.45-3.42 (m, 2H), 3.30-3.25 (m, 1H), 3.26 (s, 3H), 2.06 (d, J=10.3 Hz, 2H), 1.95 (d, J=11.3 Hz, 2H), 1.47-1.41 (m, 2H), 1.29-1.23 (m, 2H). LCMS: (Method C) 427.3 (M+H)

### Example 12: 5-(1H-imidazol-1-yl)-N7-((1r,4r)-4-(2-methoxyethoxy)cyclohexyl)-N3-methyl-1H-indole-3,7-dicarboxamide

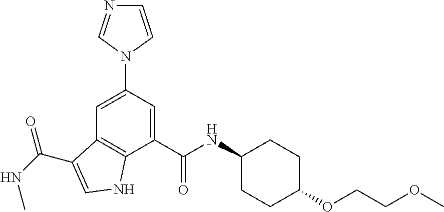 [SMILES: CNC(=O)c1c[nH]c2c(C(=O)N[C@H]3CC[C@H](OCCOC)CC3)cc(-n3ccnc3)cc12]

To a stirred solution of 5-(1H-imidazol-1-yl)-7-(((1r,4r)-4-(2-methoxyethoxy)cyclohexyl) carbamoyl)-1H-indole-3-carboxylic acid (0.08 g, 0.19 mmol) in DMF (2 mL) at 0° C., were added DIPEA (0.1 mL, 5.62 mmol) and HATU (0.107 g, 2.81 mmol) and the reaction mixture was stirred for 5 min at RT. Then methylamine hydrochloride (18.8 mg, 2.81 mmol) was added at RT and the reaction mixture was stirred at RT for 5 h. After completion (monitored by TLC), the reaction mixture was concentrated under reduced pressure and the crude residue was purified by Prep-HPLC (Method A) to get the title compound. Yield: 13% (10.97 mg, white solid). ¹H NMR (400 MHz, DMSO-d₆): δ 11.73 (s, 1H), 8.46 (d, J=7.2 Hz, 1H), 8.41 (s, 1H), 8.15 (s, 1H), 8.10 (s, 1H), 8.06 (d, J=4.8 Hz, 1H), 7.92 (s, 1H), 7.69 (d, J=1.2 Hz, 1H), 7.14 (d, J=0.8 Hz, 1H), 3.91-3.82 (m, 1H), 3.58-3.54 (m, 2H), 3.44-3.41 (m, 2H), 3.30-3.24 (m, 4H), 2.78 (d, J=4.4 Hz, 3H), 2.06 (d, J=10.4 Hz, 2H), 1.94 (d, J=12.0 Hz, 2H), 1.47-1.37 (m, 2H), 1.32-1.23 (m, 2H). LCMS: (Method C) 440.3 (M+H)

### Example 13: 5-(1H-imidazol-1-yl)-N7-((1r,4r)-4-(2-methoxyethoxy)cyclohexyl)-N3,N3-dimethyl-1H-indole-3,7-dicarboxamide

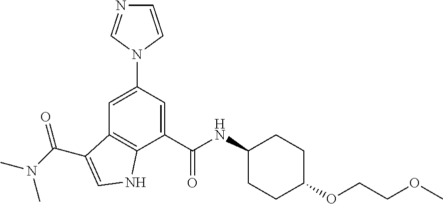

To a stirred solution of 5-(1H-imidazol-1-yl)-7-(((1r,4r)-4-(2-methoxyethoxy)cyclohexyl) carbamoyl)-1H-indole-3-carboxylic acid (0.08 g, 0.19 mmol) in DMF (2 mL) at 0° C., were added DIPEA (0.1 mL, 0.58 mmol) and HATU (0.107 g, 0.28 mmol). After stirring for 5 min at 0° C., dimethylamine hydrochloride (23 mg, 0.28 mmol) was added and the reaction mixture was stirred at RT for 5 h. After completion (monitored by TLC), the reaction mixture was concentrated under reduced pressure and the resulting crude residue was purified by Prep-HPLC (Method A) to get the title compound. Yield: 21% (18.61 mg, white solid). ¹H NMR (400 MHz, DMSO-d₆): δ 11.71 (s, 1H), 8.48 (d, J=7.6 Hz, 1H), 8.18 (s, 1H), 8.08 (d, J=1.6 Hz, 1H), 7.93 (d, J=2.0 Hz, 1H), 7.76 (d, J=2.8 Hz, 1H), 7.72-7.71 (m, 1H), 7.13 (s, 1H), 3.89-3.85 (m, 1H), 3.56-3.54 (m, 2H), 3.45-3.42 (m, 2H), 3.30-3.24 (m, 4H), 3.11 (s, 6H), 2.06 (d, J=10.4 Hz, 2H), 1.95 (d, J=11.6 Hz, 2H), 1.47-1.39 (m, 2H), 1.31-1.23 (m, 2H). LCMS: (Method C) 454.2 (M+H)

### Example 14: 5-(1H-imidazol-1-yl)-N-((1r,4r)-4-(2-methoxyethoxy)cyclohexyl)-3-nitro-1H-indole-7-carboxamide

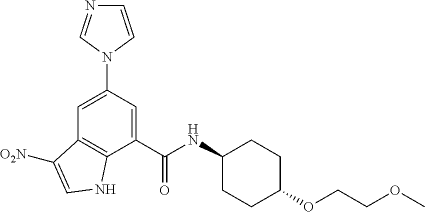 [SMILES: COCCO[C@H]1CC[C@H](NC(=O)c2cc(-n3ccnc3)cc3c([N+](=O)[O-])c[nH]c23)CC1]

To a stirred solution of N-bromosuccinimide (251 mg, 1.41 mmol) and AgNO₃ (300 mg, 1.76 mmol) in acetonitrile (20 mL) at 80° C. was added 5-(1H-imidazol-1-yl)-N-((1r,4r)-4-(2-methoxyethoxy)cyclohexyl)-1H-indole-7-carboxamide (0.45 g, 1.18 mmol) slowly and the reaction mixture was stirred for 16 h at 80° C. After completion (monitored by TLC), the reaction mixture was cooled to RT, filtered through Celite and the Celite bed was washed with DCM (25 mL). The combined filtrate was concentrated under reduced pressure. The resulting crude residue was purified by flash chromatography on silica gel (230-400 mesh silica gel, eluent: 4% MeOH in DCM) followed by further purification by Prep-HPLC (Method A) to get the title compound. Yield: 1% (5 mg, off white solid). ¹H NMR (400 MHz, DMSO-d₆): δ 10.48 (s, 1H), 8.37 (s, 1H), 8.19 (br s, 1H), 8.13 (br s, 2H), 7.81 (s, 1H), 7.69 (s, 1H), 7.12 (s, 1H), 3.87 (br s, 1H), 3.59-3.53 (m, 2H), 3.45-3.42 (m, 2H), 3.30-3.22 (s, 4H), 2.05-1.97 (m, 4H), 1.44-1.28 (m, 4H). LCMS: (Method C) 428.3 (M+H)

### Example 15: 3-formyl-5-(1H-imidazol-1-yl)-N-((1r,4r)-4-(2-methoxyethoxy)cyclohexyl)-1H-indole-7-carboxamide

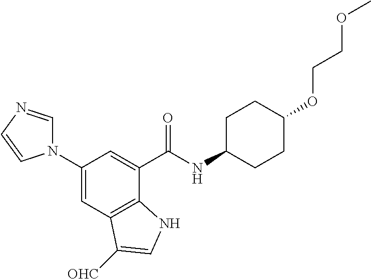 [SMILES: COCCO[C@H]1CC[C@H](NC(=O)c2cc(-n3ccnc3)cc3c(C=O)c[nH]c23)CC1]

A mixture of POCl₃ (0.72 g, 4.71 mmol) and DMF (1.14 g, 15.71 mmol) was stirred at RT for 30 min. Then a solution of 5-(1H-imidazol-1-yl)-N-((1r,4r)-4-(2-methoxyethoxy)cyclohexyl)-1H-indole-7-carboxamide (1.5 g, 3.92 mmol) in DMF (6 mL) was slowly added at 0° C. over 5 min and the reaction mixture was stirred at RT for 4 h. After completion (monitored by TLC), the reaction mixture was quenched with water (10 mL) and diluted with 10% MeOH in DCM (2×25 mL). The combined organic layer was dried over anhydrous Na₂SO₄, filtered and the filtrate was concentrated under vacuum. The resulting crude residue was purified by flash chromatography on Biotage Isolera (silica gel 230-400 mesh, eluent: 4% MeOH in DCM) followed by further purification by Prep-HPLC (Method A) to get the title compound. Yield: 31% (500 mg, off white solid) ¹H NMR (400 MHz, CD₃OD): δ 10.01 (s, 1H), 8.50 (d, J=2.0 Hz, 1H), 8.31 (s, 1H), 8.23 (s, 1H), 7.99 (d, J=2.0 Hz, 1H), 7.68 (s, 1H), 7.22 (s, 1H), 4.04-3.96 (m, 1H), 3.68-3.66 (m, 2H), 3.57-3.54 (m, 2H), 3.39-3.30 (m, 4H), 2.20-2.09 (m, 4H), 1.53-1.40 (m, 4H). LCMS: (Method C) 411.3 (M+H)

### Example 16: 3-((dimethylamino)methyl)-5-(1H-imidazol-1-yl)-N-((1r,4r)-4-(2-methoxyethoxy)cyclohexyl)-1H-indole-7-carboxamide

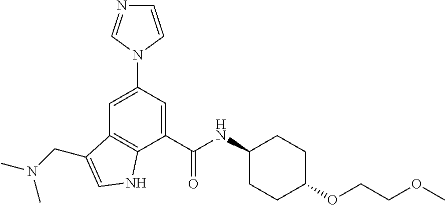

To a stirred solution of 3-formyl-5-(1H-imidazol-1-yl)-N-((1r,4r)-4-(2-methoxyethoxy)cyclohexyl)-1H-indole-7-carboxamide (0.10 g, 0.24 mmol) and dimethylamine hydrochloride (39 mg, 0.49 mmol) in methanol (5 mL) at RT was added 1 drop of acetic acid and the reaction mixture was stirred for 3 h at RT. Then sodium triacetoxyborohydride (155 mg, 0.73 mmol) was added and the reaction mixture was stirred at RT for 16 h while being monitored by LCMS. After 16 h, sodium borohydride (27 mg, 0.73 mmol) was added at 0° C., and the reaction mixture was stirred at RT for 3 d. The reaction mixture was concentrated under reduced pressure. The residue was diluted with 10% MeOH in DCM (25 mL) and washed with water (10 mL). The organic layer was dried over anhydrous Na₂SO₄, filtered and the filtrate was concentrated under vacuum. The resulting crude residue was purified by Prep-HPLC (Method A). The prep fraction was concentrated under reduced pressure, the residue was diluted with 10% MeOH in DCM (10 mL) and neutralized with sat. aq. NaHCO₃ solution. The organic layer was dried over anhydrous Na₂SO₄, filtered, concentrated under reduced pressure and finally lyophilized to get the title compound. Yield: 13% (14.04 mg, white solid). ¹H NMR (400 MHz, DMSO-d₆): δ 11.12 (s, 1H), 8.36 (d, J=8.0 Hz, 1H), 8.14 (s, 1H), 7.91 (s, 1H), 7.84 (s, 1H), 7.68 (d, J=0.8 Hz, 1H), 7.31 (d, J=1.6 Hz, 1H), 7.11 (s, 1H), 3.89-3.81 (m, 1H), 3.58 (s, 2H), 3.55-3.53 (m, 2H), 3.43-3.41 (m, 2H), 3.30-3.26 (m, 4H), 2.15 (s, 6H), 2.06-2.03 (m, 2H), 1.94-1.91 (m, 2H), 1.45-1.36 (m, 2H), 1.30-1.21 (m, 2H). LCMS: (Method B) 440.2 (M+H)

### Example 17: 3-(aminomethyl)-5-(1H-imidazol-1-yl)-N-((1r,4r)-4-(2-methoxyethoxy)cyclohexyl)-1H-indole-7-carboxamide

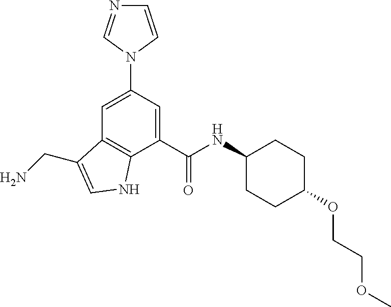 [SMILES: COCCO[C@H]1CC[C@H](NC(=O)c2cc(-n3ccnc3)cc3c(CN)c[nH]c23)CC1]

To a stirred solution of 3-cyano-5-(1H-imidazol-1-yl)-N-((1r,4r)-4-(2-methoxyethoxy)cyclohexyl)-1H-indole-7-carboxamide (0.25 g, 0.61 mmol) and nickel chloride hexahydrate (0.792 g, 6.14 mmol) in methanol (10 mL) at 0° C., sodium borohydride (0.116 g, 3.06 mmol) was added slowly and the reaction mixture was stirred at RT for 16 h. After completion (monitored by TLC), the reaction mixture was concentrated under vacuum. The crude residue was dissolved in 20% MeOH in DCM (25 mL), filtered through Celite and the Celite bed was washed with 20% MeOH in DCM (50 mL). The filtrate was concentrated under vacuum. The resulting crude residue was purified by Prep-HPLC (Method A). The fraction was concentrated under reduced pressure, diluted with 10% MeOH in DCM (10 mL) and neutralized with sat. aq. NaHCO₃ solution. The organic layer was dried over anhydrous Na₂SO₄, filtered, concentrated under reduced pressure and lyophilized to get the title compound. Yield: 5% (11.74 mg, off white solid). ¹H NMR (400 MHz, DMSO-d₆): δ 11.44 (s, 1H), 8.45 (d, J=7.6 Hz, 1H), 8.20-8.18 (m, 2H), 7.96-7.83 (m, 3H), 7.73 (s, 1H), 7.58 (d, J=2.0 Hz, 1H), 7.16 (s, 1H), 4.25 (s, 2H), 3.91-3.82 (m, 1H), 3.57-3.53 (m, 2H), 3.44-3.42 (m, 2H), 3.30-3.25 (m, 4H), 2.08-2.07 (m, 2H), 1.96-1.92 (m, 2H), 1.44-1.38 (m, 2H), 1.31-1.24 (m, 2H). LCMS: (Method A) 412.3 (M+H)

### Example 18: 3-(hydroxymethyl)-5-(1H-imidazol-1-yl)-N-((1r,4r)-4-(2-methoxyethoxy)cyclohexyl)-1H-indole-7-carboxam

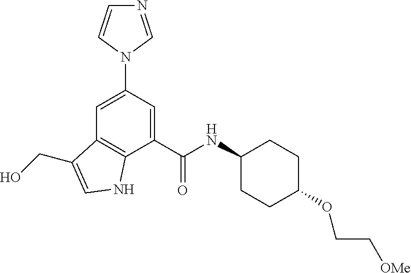 [SMILES: COCCO[C@H]1CC[C@H](NC(=O)c2cc(-n3ccnc3)cc3c(CO)c[nH]c23)CC1]

To a stirred solution 3-formyl-5-(1H-imidazol-1-yl)-N-((1r,4r)-4-(2-methoxyethoxy)cyclohexyl)-1H-indole-7-carboxamide (80 mg, 0.19 mmol) in MeOH (3 mL), NaBH₄ (11 mg, 0.29 mmol) was added at 0° C. under nitrogen atmosphere and stirred at RT 6 h. Reaction was monitoring by TLC, starting material was consumed. The reaction mixture was quenched with water (5 mL) and extracted with 10% MeOH in DCM (20 mL). The organic layer was evaporated under vacuum and the resulting crude was purified by Prep-HPLC (Method B). The Prep. HPLC fraction was concentrated and resulting residue was dissolved in 10% MeOH in DCM (10 mL), washed with water, dried over anhydrous Na₂SO₄, filtered, concentrated under reduced pressure and lyophilized to get title compound. Yield: 20% (16.05 mg, off white solid). ¹HNMR (400 MHz, DMSO-d₆): 11.09 (s, 1H), 8.38 (d, J=7.7 Hz, 1H), 8.16 (d, J=1.0 Hz, 1H), 7.95 (d, J=1.8 Hz, 1H), 7.87 (d, J=1.9 Hz, 1H), 7.71-7.68 (m, 1H), 7.40 (d, J=2.4 Hz, 1H), 7.13-7.10 (m, 1H), 4.90 (t, J=5.7 Hz, 1H), 4.70 (d, J=5.6 Hz, 2H), 3.87-3.84 (m, 1H), 3.60-3.56 (m, 2H), 3.44-3.40 (m, 2H), 3.35-3.30 (m, 4H), 2.07-1.95 (m, 4H), 1.46-1.40 (m, 4H). LCMS: (Method C) 413.3 (M+H)

### Example 19: 5-(1H-imidazol-1-yl)-N-((1r,4r)-4-(2-methoxyethoxy)cyclohexyl)-3-(trifluoromethyl)-1H-indole-7-carboxamide

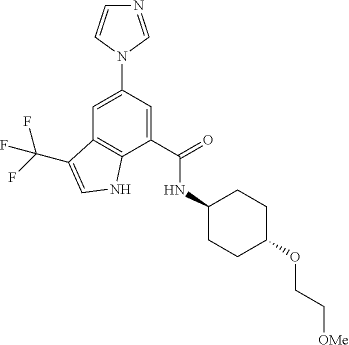 [SMILES: COCCO[C@H]1CC[C@H](NC(=O)c2cc(-n3ccnc3)cc3c(C(F)(F)F)c[nH]c23)CC1]

To a stirred solution of 5-(1H-imidazol-1-yl)-3-iodo-N-((1r,4r)-4-(2-methoxyethoxy)cyclohexyl)-1H-indole-7-carboxamide (0.4 g, 0.78 mmol) in dry DCM (5 mL), FeCl₂ (9.97 mg, 0.07 mmol) and Togni II reagent (0.49 mg, 1.57 mmol) were added at RT. Then the reaction mixture was stirred at RT for 48 h. After completion, the reaction mixture was filtered through Celite and washed with 10% MeOH in DCM (100 mL). The combined filtrate was evaporated under vacuum and the resulting crude was purified by Prep-HPLC (Method B). The prep-fraction was collected, concentrated under reduced pressure, dissolved in 10% MeOH/DCM (10 mL) and washed with water. The organic layer was dried over anhydrous Na₂SO₄, concentrated under reduced pressure and lyophilized to get the title compound. Yield: 8% (30 mg, off white solid). ¹HNMR (400 MHz, DMSO-d₆): 12.14 (s, 1H), 8.54 (d, J=8.0 Hz, 1H), 8.23 (s, 1H), 8.08 (s, 1H), 7.99 (s, 1H), 7.76 (s, 1H), 7.14 (s, 2H), 3.87-3.85 (m, 1H), 3.56-3.55 (m, 2H), 3.44-3.43 (m, 2H), 3.34-3.29 (m, 4H), 2.07-2.02 (m, 2H), 1.98-1.95 (m, 2H), 1.45-1.39 (m, 2H), 1.24-1.22 (m, 2H). LCMS: (Method A) 451.2 (M+H).

### Example 20: 3-cyano-N-((1r,4r)-4-(2-methoxyethoxy)cyclohexyl)-5-(thiazol-5-yl)-1H-indole-7-carboxamide

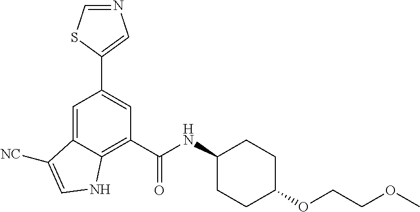 [SMILES: COCCO[C@H]1CC[C@H](NC(=O)c2cc(-c3cncs3)cc3c(C#N)c[nH]c23)CC1]

To a stirred solution of N-((1r,4r)-4-(2-methoxyethoxy)cyclohexyl)-5-(thiazol-5-yl)-1H-indole-7-carboxamide (0.1 g, 0.25 mmol) in acetonitrile (2.0 mL) at 0° C., chlorosulfonyl isocyanate (0.18 g, 1.27 mmol) was added slowly over 2 min. After complete addition, the reaction mixture was stirred for 30 min at 0° C., then DMF (0.183 g, 2.50 mmol) was added slowly over 2 min. The reaction mixture was stirred at 0° C. for 30 min and at RT for 16 h. After completion (monitored by TLC), the reaction mixture was quenched with water (2.0 mL), then stirred for 15 min at RT and extracted with EtOAc (3×5 mL). The combined organic layer was washed with water (5 mL) followed by brine (5 mL) and dried over anhydrous Na₂SO₄. The organic layer was filtered and concentrated under vacuum. The crude residue was purified by flash chromatography on Biotage Isolera (silica gel: 100-200 mesh, eluent: 0-10% MeOH in DCM). The material obtained was further purified by Prep-HPLC (Method B). Yield: 20% (21 mg, off white solid). ¹H NMR (400 MHz, DMSO-d₆): δ 12.25 (s, 1H), 9.12 (s, 1H), 8.65 (br s, 1H), 8.42 (s, 1H), 8.23 (s, 1H), 8.11 (s, 1H), 8.03 (s, 1H), 3.93-3.83 (m, 1H), 3.57-3.54 (m, 2H), 3.45-3.42 (m, 2H), 3.31-3.26 (m, 4H), 2.08-2.04 (m, 2H), 1.97-1.94 (m, 2H), 1.47-1.40 (m, 2H), 1.31-1.23 (m, 2H). LCMS: (Method C) 425.0 (M+H).

### Example 21: 3-cyano-5-(1H-imidazol-1-yl)-N-((1r, 4r)-4-(2-methoxyethoxy)cyclohexyl)-1H-indole-7-carboxamide

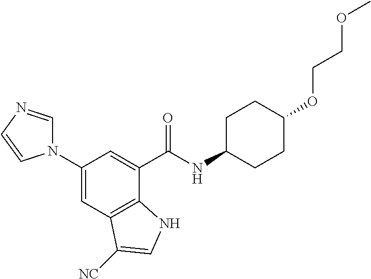 [SMILES: COCCO[C@H]1CC[C@H](NC(=O)c2cc(-n3ccnc3)cc3c(C#N)c[nH]c23)CC1]

To a stirred solution of 5-(1H-imidazol-1-yl)-N-((1r,4r)-4-(2-methoxyethoxy)cyclohexyl)-1H-indole-7-carboxamide (350 mg, 0.916 mmol) in ACN (10.0 mL) chlorosulfonyl isocyanate (0.079 mL, 0.916 mmol) was added slowly over 2 min at 0° C. and after stirring for 30 min DMF (0.073 mL, 1.00 mmol) was added at same temperature. Further stirring was continued for 30 min at same temperature and at RT for 3 h. Reaction was monitored by TLC, starting material was consumed. The reaction mixture was quenched with water (3.0 mL) and stirred for 15 min. The resulting reaction mixture was evaporated in vacuum to get crude compound which was purified by flash chromatography (Biotage Isolera, 230-400 mesh silica gel, 0-10% MeOH in DCM) to afford the title compound Yield: 47% (0.175 g, Off white solid). ¹H NMR (400 MHz, DMSO-d₆): δ 12.28 (s, 1H), 8.56 (d, J=7.72 Hz, 1H), 8.34 (s, 1H), 8.28 (s, 1H), 8.05 (s, 2H), 7.87 (s, 1H), 7.16 (s, 1H), 3.88-3.84 (m, 1H), 3.56-3.53 (m, 2H), 3.44-3.41 (m, 2H), 3.26-3.25 (m, 4H), 2.07-2.04 (m, 2H), 1.96-1.93 (m, 2H), 1.46-1.37 (m, 2H), 1.31-1.22 (m, 2H). LCMS: (Method C) 408.3 (M+H),

### Example 22: 5-(1H-imidazol-1-yl)-3-iodo-N-((1r,4r)-4-(2-methoxyethoxy)cyclohexyl)-1H-indole-7-carboxamide

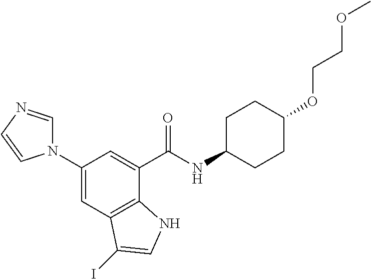 [SMILES: COCCO[C@H]1CC[C@H](NC(=O)c2cc(-n3ccnc3)cc3c(I)c[nH]c23)CC1]

To a stirred solution of 5-(1H-imidazol-1-yl)-N-((1r,4r)-4-(2-methoxyethoxy)cyclohexyl)-1H-indole-7-carboxamide (500 mg, 1.30 mmol) in ACN (5.0 mL) was added NIS (320 mg, 1.43 mmol) at −10° C. and stirred at same temperature for 4 h at −12° C. Reaction was monitored by TLC, starting material was consumed. The reaction mixture was quenched with 10% Na₂S203 (5.0 mL). The suspension was stirred for 15 min at 0° C. and solids were collected by filtrations to afford the title compound which was purified by flash chromatography (Biotage Isolera, 100-200 mesh silica gel, 0-10% MeOH in DCM) to afford the title compound Yield: 74% (490 mg, White solid). ¹H NMR (400 MHz, DMSO-d₆): δ 11.65 (s, 1H), 8.19 (s, 1H), 7.88 (s, 1H), 7.45 (s, 1H), 7.56-7.52 (m, 2H), 7.12 (s, 1H), 3.86-3.85 (m, 1H), 3.54-3.53 (m, 2H), 3.44-3.42 (m, 2H), 3.26-3.25 (m, 4H), 2.05-2.03 (m, 2H), 1.96-1.93 (m, 2H), 1.42-1.36 (m, 2H), 1.28-1.25 (m, 2H). LCMS: (Method C) 509.0 (M+H)

### Example 23: 5-(1H-imidazol-1-yl)-N7-((1r,4r)-4-(2-methoxyethoxy)cyclohexyl)-1H-indole-3,7-dicarboxamide

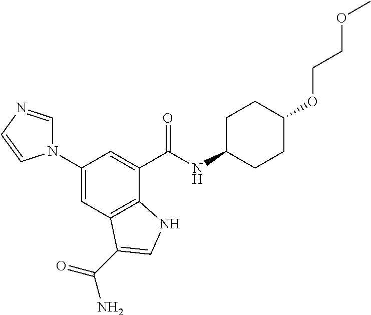 [SMILES: COCCO[C@H]1CC[C@H](NC(=O)c2cc(-n3ccnc3)cc3c(C(N)=O)c[nH]c23)CC1]

A mixture of 3-cyano-5-(1H-imidazol-1-yl)-N-((1r,4r)-4-(2-methoxyethoxy)cyclohexyl)-1H-indole-7-carboxamide (150 mg, 0.368 mmol) in EtOH (8.0 mL), 3N NaOH solution (12.28 mL, 36.8 mmol) and H₂O₂ (4.17 mL, 36.8 mmol) were added at RT and the reaction mixture as heated at 78° C. for 48 h. After completion the reaction, the reaction mixture was concentrated under vacuum and co-distilled with MeOH twice. The resulting crude was purified by flash chromatography (Biotage Isolera, 230-400 mesh silica gel, 0-15% MeOH in DCM) to afford the title compound Yield: 41% (63.6 mg, White solid). ¹HNMR (400 MHz, DMSO-d₆): δ 11.76 (s, 1H), 8.47 (d, J=6.5 Hz, 1H), 8.39 (s, 1H), 8.17-8.15 (m, 2H), 7.91 (s, 1H), 7.68 (s, 1H), 7.61 (br s, 1H), 7.13 (s, 1H), 6.95 (br s, 1H), 3.87-3.85 (m, 1H), 3.56-3.54 (m, 2H), 3.44-3.42 (m, 2H), 3.26-3.25 (s, 4H), 2.07-2.04 (m, 2H), 1.96-1.93 (m, 2H), 1.46-1.37 (m, 2H), 1.31-1.22 (m, 2H). LCMS: (Method C) 426.1 (M+H)

### Example 24: N-(1,1-dioxidotetrahydro-2H-thiopyran-4-yl)-6-(thiazol-5-yl)-1H-indole-4-carboxamide

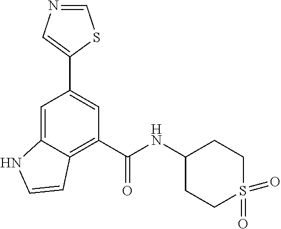 [SMILES: O=C(NC1CCS(=O)(=O)CC1)c1cc(-c2cncs2)cc2[nH]ccc12]

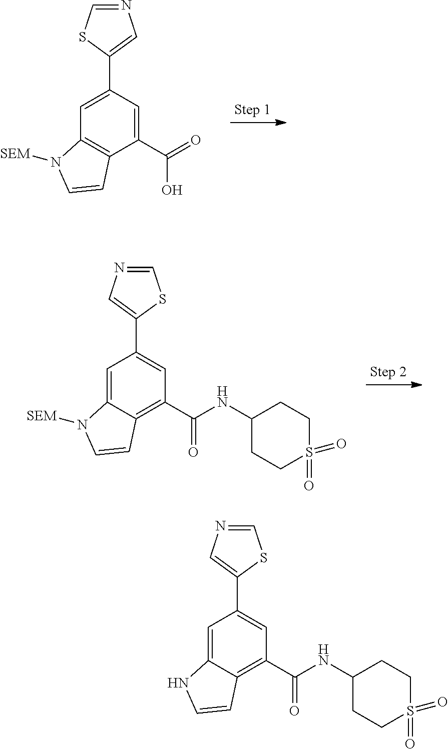 [SMILES: C[Si](C)(C)CCOCn1ccc2c(C(=O)O)cc(-c3cncs3)cc21 ; C[Si](C)(C)CCOCn1ccc2c(C(=O)NC3CCS(=O)(=O)CC3)cc(-c3cncs3)cc21 ; O=C(NC1CCS(=O)(=O)CC1)c1cc(-c2cncs2)cc2[nH]ccc12]

### Step 1: N-(1,1-dioxidotetrahydro-2H-thiopyran-4-yl)-6-(thiazol-5-yl)-1-((2-(trimethylsilyl) ethoxy) methyl)-1H-indole-4-carboxamide

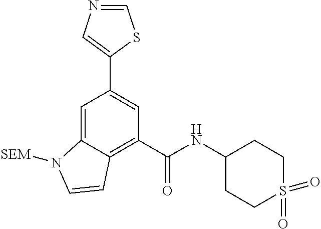 [SMILES: C[Si](C)(C)CCOCn1ccc2c(C(=O)NC3CCS(=O)(=O)CC3)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (5 mL) at 0° C. HATU (0.30 g, 0.80 mmol) and DIPEA (0.24 mL, 1.33 mmol) were added under nitrogen atmosphere and stirred for 5 min. Then 4-aminotetrahydro-2H-thiopyran 1,1-dioxide (0.09 g, 0.64 mmol) was added at same temperature and stirring was continued at RT for 16 h. After completion of reaction, the reaction mixture was diluted with ice cold water (10 mL) and extracted with EtOAc (50 mL). The organic solution was washed with water (25 mL), brine solution (50 mL), dried over anhydrous Na₂SO₄ and concentrated under vacuum. The resulting crude product was used for next step without purification. Yield: 74% (0.2 g, Brown gummy solid). LCMS: (Method C) 506.2 (M+H)

### Step 2: N-(1,1-dioxidotetrahydro-2H-thiopyran-4-yl)-6-(thiazol-5-yl)-1H-indole-4-carboxamide

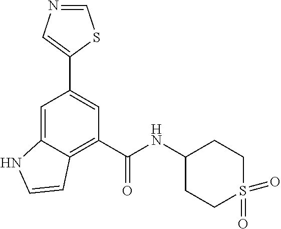 [SMILES: O=C(NC1CCS(=O)(=O)CC1)c1cc(-c2cncs2)cc2[nH]ccc12]

To a stirred solution of N-(1,1-dioxidotetrahydro-2H-thiopyran-4-yl)-6-(thiazol-5-yl)-1-((2-(trimethylsilyl)ethoxy)methyl)-1H-indole-4-carboxamide (0.2 g, 0.39 mmol) in THF (5 mL) at RT, TBAF (1.0 M in THF) (1.18 mL, 1.18 mmol) was added and stirred for 16 h at 70° C. After completion of reaction, the reaction mixture was quenched with ice cold water (10 mL) and extracted with EtOAc (100 mL). The organic solution was washed with brine solution (50 mL) dried over anhydrous Na₂SO₄, concentrated under vacuum and the resulting crude was purified by Prep-HPLC (Method A). The prep-fraction was concentrated under reduced pressure and resulting solid was dissolved in 10% MeOH in DCM (10 mL). The organic layer was washed with water, dried over anhydrous Na₂SO₄, concentrated and lyophilized to get the title compound. Yield: 8% (11.58 mg, off white solid). ¹HNMR (400 MHz, DMSO-d₆): 11.42 (s, 1H), 9.06 (s, 1H), 8.43 (d, J=7.8 Hz, 1H), 8.32 (s, 1H), 7.78 (s, 1H), 7.70 (d, J=1.3 Hz, 1H), 7.51 (d, J=2.5 Hz, 1H), 6.80 (d, J=2.5 Hz, 1H), 4.31-4.28 (m, 1H), 3.16-3.13 (m, 2H), 2.51-2.49 (m, 2H), 2.18-2.19 (m, 4H). LCMS: (Method C) 376.1 (M+H)

### Example 25: N-(2-fluoro-6-(trifluoromethyl)benzyl)-6-(thiazol-5-yl)-1H-indole-4-carboxamide

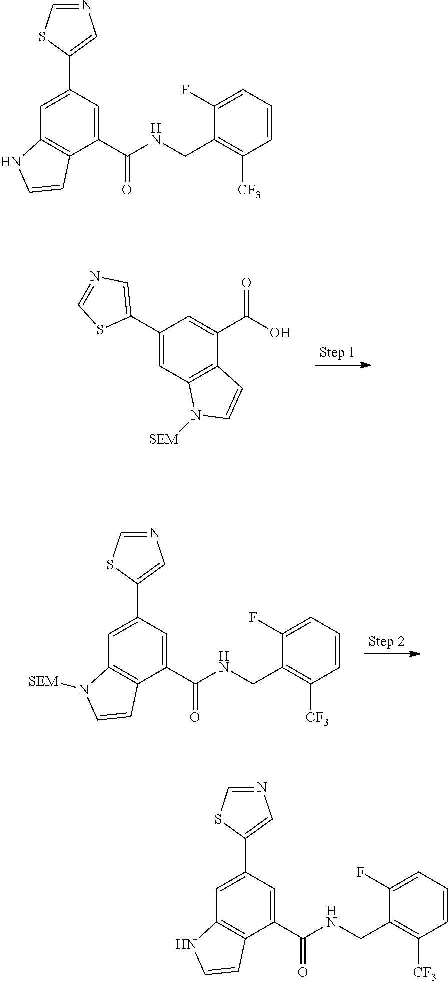 [SMILES: O=C(NCc1c(F)cccc1C(F)(F)F)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)NCc3c(F)cccc3C(F)(F)F)cc(-c3cncs3)cc21 ; O=C(NCc1c(F)cccc1C(F)(F)F)c1cc(-c2cncs2)cc2[nH]ccc12]

### Step 1: N-(2-fluoro-6-(trifluoromethyl)benzyl)-6-(thiazol-5-yl)-1-((2-(trimethylsilyl)ethoxy)methyl)-1H-indole-4-carboxamide

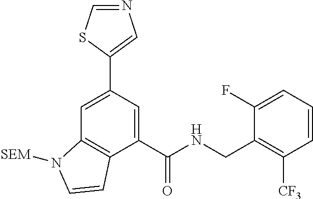 [SMILES: C[Si](C)(C)CCOCn1ccc2c(C(=O)NCc3c(F)cccc3C(F)(F)F)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (2 mL) at 0° C., HATU (304 mg, 0.80 mmol) was added followed by DIPEA (0.29 mL, 1.60 mmol) under nitrogen atmosphere. The reaction was stirred at 0° C. for 10 min and then (2-fluoro-6-(trifluoromethyl)phenyl)methanamine (123 mg, 0.64 mmol) was added and the reaction mixture was stirred for another 16 h at RT. The reaction mixture was quenched with ice cold water (10 mL) and extracted with EtOAc (2×10 mL). The resulting organic layer was washed with water (2×10 mL), brine solution (2×10 mL), dried over anhydrous Na₂SO₄, filtered and the filtrate was concentrated under vacuum. The resulting crude residue was used for next step without further purification. Yield: 82% (0.24 g, brown solid). LCMS: (Method A) 550.2 (M+H)

### Step 2: N-(2-fluoro-6-(trifluoromethyl)benzyl)-6-(thiazol-5-yl)-1H-indole-4-carboxamide

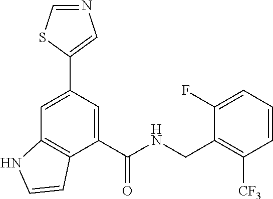 [SMILES: O=C(NCc1c(F)cccc1C(F)(F)F)c1cc(-c2cncs2)cc2[nH]ccc12]

To a stirred solution of N-(2-fluoro-6-(trifluoromethyl)benzyl)-6-(thiazol-5-yl)-1-((2-(trimethylsilyl)ethoxy)methyl)-1H-indole-4-carboxamide (240 mg, 0.43 mmol) in THF (10 mL) at RT, a solution of TBAF in THF (1.31 mL, 1.31 mmol, 1.0 M) was added slowly and the reaction mixture was heated to reflux for 16 h. After completion, the reaction mixture was cooled to RT and concentrated under reduced pressure. The crude residue was suspended with ice cold water (10 mL) and then extracted with EtOAc (2×10 mL). The resulting organic layer was washed with water (2×10 mL), brine solution (2×10 mL), dried over anhydrous Na₂SO₄, filtered and the filtrate was concentrated under vacuum. The resulting crude residue was purified by Prep-HPLC (Method A). The prep-fraction was collected, concentrated under reduced pressure and diluted with 10% MeOH in DCM (10 mL). The organic layer was washed with 10% aq. NaHCO₃ solution (4 mL), followed by brine solution and water. The organic layer was dried over anhydrous Na₂SO₄, filtered, concentrated under reduced pressure and lyophilized to get the title compound. Yield: 11% (20.4 mg, white solid). ¹H NMR (400 MHz, DMSO-d₆): δ 11.42 (s, 1H), 9.03 (s, 1H), 8.72-8.69 (m, 1H), 8.25 (s, 1H), 7.77 (s, 1H), 7.67-7.61 (m, 4H), 7.51 (d, J=2.8 Hz, 1H), 6.82 (d, J=2.8 Hz, 1H), 4.69 (d, J=3.8 Hz, 2H). LCMS: (Method A) 419.9 (M+H)

### Example 26: N-(2-(methylsulfonyl)ethyl)-6-(thiazol-5-yl)-1H-indole-4-carboxamide

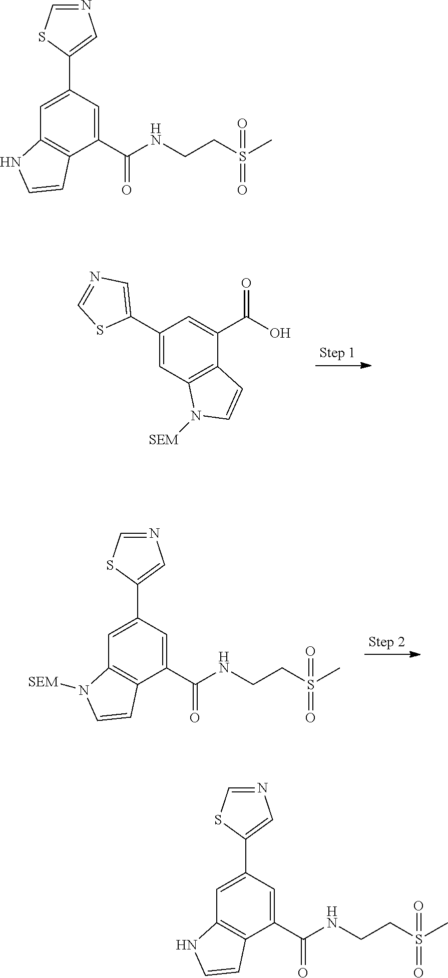 [SMILES: CS(=O)(=O)CCNC(=O)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)NCCS(C)(=O)=O)cc(-c3cncs3)cc21 ; CS(=O)(=O)CCNC(=O)c1cc(-c2cncs2)cc2[nH]ccc12]

### Step 1: N-(2-(methylsulfonyl)ethyl)-6-(thiazol-5-yl)-1-((2-(trimethylsilyl)ethoxy)methyl)-1H-indole-4-carboxamide

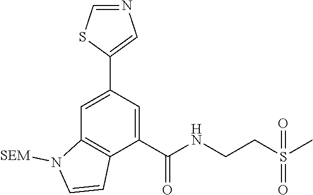 [SMILES: C[Si](C)(C)CCOCn1ccc2c(C(=O)NCCS(C)(=O)=O)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (2 mL) at 0° C., HATU (300 mg, 0.80 mmol) and DIPEA (0.206 g, 1.60 mmol) were added under nitrogen atmosphere and the reaction mixture was stirred at 0° C. for 10 min. Then 2-(methylsulfonyl)ethan-1-amine (78 mg, 0.64 mmol) was added and the reaction mixture was stirred for 16 h at RT. The reaction mixture was diluted with ice cold water (10 mL) and extracted with EtOAc (2×10 mL). The combined organic layer was washed with water (2×10 mL), brine solution (2×10 mL), dried over anhydrous Na₂SO₄, filtered and the filtrate was concentrated under vacuum. The resulting crude residue was used in the next step without further purification. Yield: 98% (0.25 g, pale yellow solid). LCMS: (Method C) 480.2 (M+H)

### Step 2: N-(2-(methylsulfonyl)ethyl)-6-(thiazol-5-yl)-1H-indole-4-carboxamide

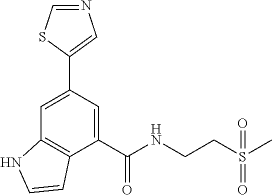 [SMILES: CS(=O)(=O)CCNC(=O)c1cc(-c2cncs2)cc2[nH]ccc12]

To a stirred solution of N-(2-(methylsulfonyl)ethyl)-6-(thiazol-5-yl)-1-((2-(trimethylsilyl)ethoxy) methyl)-1H-indole-4-carboxamide (250 mg, 0.52 mmol) in THF (10 mL), a solution of TBAF in THF (1.56 mL, 1.56 mmol, 1.0 M) was added slowly at RT and the reaction mixture was heated to reflux for 16 h. After completion, the reaction mixture was concentrated under reduced pressure. The residue was suspended with ice cold water (10 mL) and extracted with EtOAc (2×10 mL). The combined organic layer was washed with water (2×10 mL), brine solution (2×10 mL), dried over anhydrous Na₂SO₄, filtered and the filtrate was concentrated under vacuum. The crude residue was purified by Prep-HPLC (Method A). The prep fraction was concentrated under reduced pressure. The resulting residue was diluted with 10% MeOH in DCM (10 mL) and washed with 10% aq. NaHCO₃ solution (4 mL) followed by brine solution and water. The organic layer was dried over anhydrous Na₂SO₄, filtered, concentrated under reduced pressure and lyophilized to get the title compound. Yield: 11% (13.32 mg, white solid). ¹H NMR (400 MHz, DMSO-d₆): δ 11.46 (s, 1H), 9.06 (s, 1H), 8.64 (t, J=5.6 Hz, 1H), 8.30 (s, 1H), 7.81 (s, 1H), 7.75 (d, J=1.4 Hz, 1H), 7.54-7.53 (m, 1H), 6.91 (s, 1H), 3.77-3.72 (m, 2H), 3.46 (t, J=6.8 Hz, 2H), 3.08 (s, 3H). LCMS: (Method C) 350.1 (M+H)

### Example 27: N-((tetrahydro-2H-pyran-4-yl)methyl)-6-(thiazol-5-yl)-1H-indole-4-carboxamide

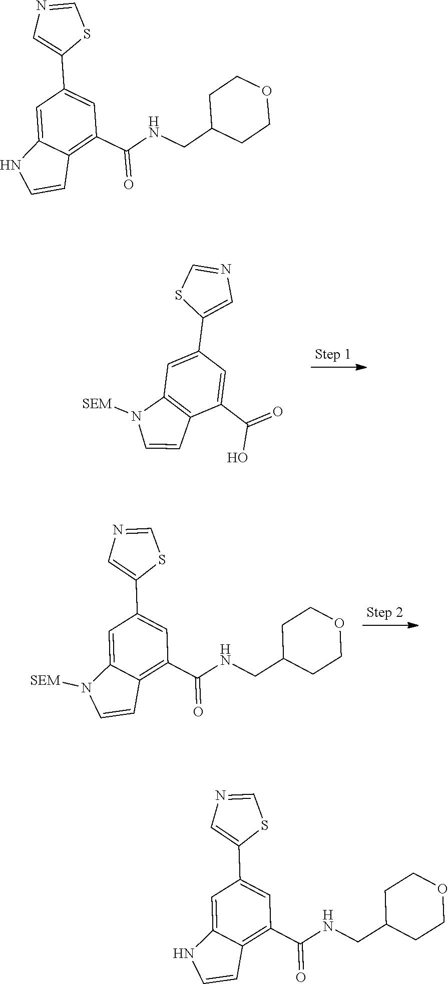 [SMILES: O=C(NCC1CCOCC1)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)NCC3CCOCC3)cc(-c3cncs3)cc21 ; O=C(NCC1CCOCC1)c1cc(-c2cncs2)cc2[nH]ccc12]

### Step 1: N-((tetrahydro-2H-pyran-4-yl)methyl)-6-(thiazol-5-yl)-1-((2-(trimethylsilyl)ethoxy) methyl)-1H-indole-4-carboxamide

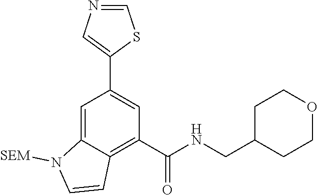 [SMILES: C[Si](C)(C)CCOCn1ccc2c(C(=O)NCC3CCOCC3)cc(-c3cncs3)cc21]

To a stirred solution of 6-(thiazol-5-yl)-1-((2-(trimethylsilyl)ethoxy)methyl)-1H-indole-4-carboxylic acid (200 mg, 0.53 mmol) in DMF (5 mL) at 0° C. HATU (304 mg, 0.80 mmol) and DIPEA (0.24 mL, 1.33 mmol) were added under nitrogen atmosphere at 0° C. and after 5 min (tetrahydro-2H-pyran-4-yl)methanamine (74 mg, 0.64 mmol) was added. Then the reaction mixture was stirred for 16 h at RT. The reaction mixture was quenched with ice cold water (10 mL) and then extracted with EtOAc (50 mL). The organic solution was washed with brine solution (50 mL), water (25 mL) dried over anhydrous Na₂SO₄ and concentrated under vacuum. The resulting crude product was used for next step without further purification. Yield: 99% (250 mg, Brown gummy solid). LCMS: (Method C) 472.2 (M+H)

### Step 2: N-((tetrahydro-2H-pyran-4-yl)methyl)-6-(thiazol-5-yl)-1H-indole-4-carboxamide

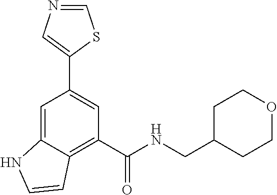 [SMILES: O=C(NCC1CCOCC1)c1cc(-c2cncs2)cc2[nH]ccc12]

To a stirred solution of N-((tetrahydro-2H-pyran-4-yl)methyl)-6-(thiazol-5-yl)-1-((2-(trimethylsilyl)ethoxy)methyl)-1H-indole-4-carboxamide (0.20 g, 0.42 mmol) in THF (5 mL) at RT, TBAF (1.0 M in THF) (1.27 mL, 1.27 mmol) was added and stirred for 16 h at 70° C. The reaction mixture was quenched with 10% NaHCO₃ solution (50 mL) and then extracted with EtOAc (100 mL). The organic solution was washed with brine solution (50 mL), dried over anhydrous Na₂SO₄, and concentrated under vacuum. The resulting crude was purified by Prep-HPLC (Method A). The prep-fraction was collected, concentrated under reduced pressure, diluted with 10% MeOH in DCM (10 mL) and washed with water. The organic layer was dried over anhydrous Na₂SO₄, concentrated under reduced pressure and lyophilized to get the title compound. Yield: 16% (22.88 mg, off white solid). ¹HNMR (400 MHz, DMSO-d₆): 11.40 (s, 1H), 9.06 (s, 1H), 8.43 (d, J=6.0 Hz, 1H), 8.31 (s, 1H), 7.77 (s, 1H), 7.74 (d, J=1.6 Hz, 1H), 7.5 (t, J=2.8 Hz, 1H), 6.80 (s, 1H), 3.88-3.85 (m, 2H), 3.34-3.32 (m, 4H), 1.88-1.86 (m, 1H), 1.67-1.64 (m, 2H), 1.30-1.27 (m, 2H). LCMS: (Method C) 342.2 (M+H)

### Example 28: N-((1r,3s,5R,7S)-3-hydroxyadamantan-1-yl)-6-(thiazol-5-yl)-1H-indole-4-carboxamide

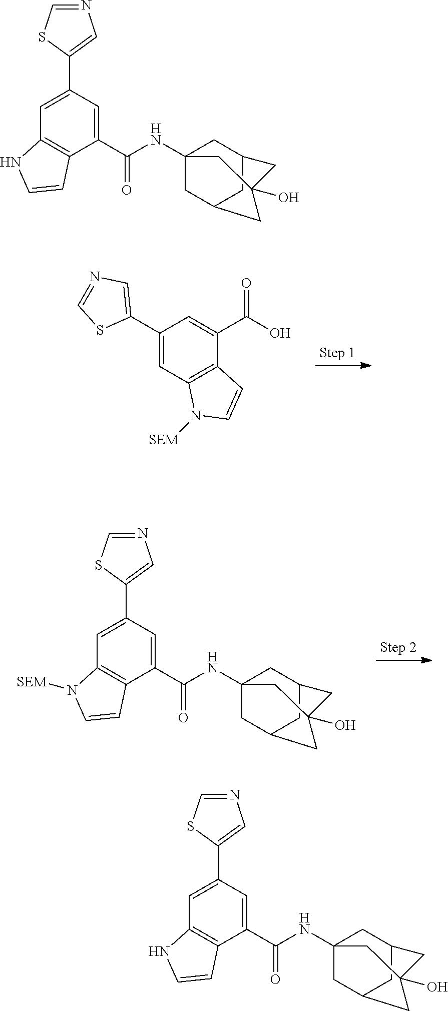 [SMILES: O=C(NC12CC3CC(CC(O)(C3)C1)C2)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)NC34CC5CC(CC(O)(C5)C3)C4)cc(-c3cncs3)cc21 ; O=C(NC12CC3CC(CC(O)(C3)C1)C2)c1cc(-c2cncs2)cc2[nH]ccc12]

### Step 1: N-((1r,3s,5R,7S)-3-hydroxyadamantan-1-yl)-6-(thiazol-5-yl)-1-((2-(trimethylsilyl)ethoxy)methyl)-1H-indole-4-carboxamide

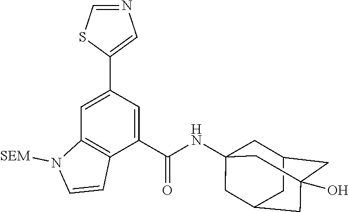 [SMILES: C[Si](C)(C)CCOCn1ccc2c(C(=O)NC34CC5CC(CC(O)(C5)C3)C4)cc(-c3cncs3)cc21]

To a stirred solution of 6-(thiazol-5-yl)-1-((2-(trimethylsilyl)ethoxy)methyl)-1H-indole-4-carboxylic acid (200 mg, 0.53 mmol) in DMF (2 mL), HATU (300 mg, 0.80 mmol) was added at 0° C. followed by DIPEA (0.206 g, 1.60 mmol) under nitrogen atmosphere and the reaction mixture was stirred at 0° C. for 10 min. Then 3-aminoadamantan-1-ol (107 mg, 0.64 mmol) was added and the reaction mixture was stirred for 16 h at RT. The reaction mixture was diluted with ice cold water (10 mL) and extracted with EtOAc (2×10 mL). The combined organic layer was washed with water (2×10 mL), brine solution (2×10 mL), dried over anhydrous Na₂SO₄, filtered and the filtrate was concentrated under vacuum. The resulting crude residue was used for next step without further purification. Yield: 86% (0.24 g, brown gum). LCMS: (Method C) 524.10 (M+H).

### Step 2: N-((1r,3s,5R,7S)-3-hydroxyadamantan-1-yl)-6-(thiazol-5-yl)-1H-indole-4-carboxamide

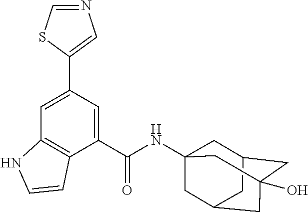 [SMILES: O=C(NC12CC3CC(CC(O)(C3)C1)C2)c1cc(-c2cncs2)cc2[nH]ccc12]

To a stirred solution of N-((1r,3s,5R,7S)-3-hydroxyadamantan-1-yl)-6-(thiazol-5-yl)-1-((2-(trimethylsilyl)ethoxy)methyl)-1H-indole-4-carboxamide (240 mg, 0.46 mmol) in THF (10 mL), a solution of TBAF in THF (1.37 mL, 1.0 M, 1.37 mmol) was added slowly at RT and the reaction mixture was heated to reflux for 16 h. After completion, the reaction mixture was concentrated under reduced pressure. The residue was diluted with ice cold water (10 mL) and extracted with EtOAc (2×10 mL). The combined organic layer was washed with water (2×10 mL), brine solution (2×10 mL), dried over anhydrous Na₂SO₄, filtered and the filtrate was concentrated under vacuum. The crude residue was purified by Prep-HPLC (Method A). The prep fraction was concentrated under reduced pressure, the residue was diluted with 10% MeOH in DCM (10 mL) and washed with 10% aq. NaHCO₃ solution (4 mL) followed by brine solution and water. The organic layer was dried over anhydrous Na₂SO₄, filtered, concentrated under reduced pressure and lyophilized to get the title compound. Yield: 19% (35.1 mg, off white solid). ¹H NMR (400 MHz, DMSO-d₆): δ 11.39 (s, 1H), 9.05 (s, 1H), 8.31 (s, 1H), 7.74 (s, 1H), 7.68 (s, 1H), 7.59 (d, J=1.6 Hz, 1H), 7.49 (d, J=2.8 Hz, 1H), 6.73 (d, J=2.8 Hz, 1H), 4.53 (s, 1H), 2.20 (br s, 2H), 2.08-1.98 (m, 6H), 1.63-1.50 (m, 6H). LCMS: (Method C) 394.2 (M+H)

### Example 29: N-((1r,4r)-4-(2-hydroxypropan-2-yl)cyclohexyl)-6-(thiazol-5-yl)-1H-indole-4-carboxamide

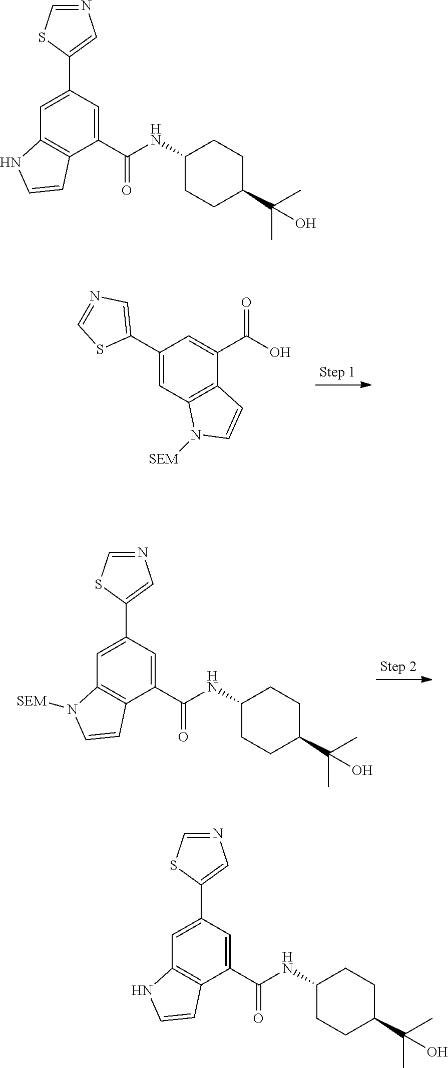 [SMILES: CC(C)(O)[C@H]1CC[C@H](NC(=O)c2cc(-c3cncs3)cc3[nH]ccc23)CC1 ; C[Si](C)(C)CCOCn1ccc2c(C(=O)O)cc(-c3cncs3)cc21 ; CC(C)(O)[C@H]1CC[C@H](NC(=O)c2cc(-c3cncs3)cc3c2ccn3COCC[Si](C)(C)C)CC1 ; CC(C)(O)[C@H]1CC[C@H](NC(=O)c2cc(-c3cncs3)cc3[nH]ccc23)CC1]

### Step 1: N-((1r,4r)-4-(2-hydroxypropan-2-yl)cyclohexyl)-6-(thiazol-5-yl)-1-((2-(trimethylsilyl)ethoxy)methyl)-1H-indole-4-carboxamide

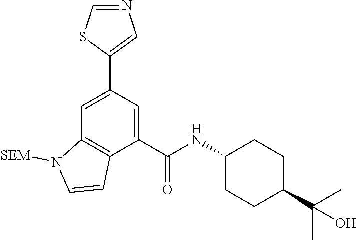 [SMILES: CC(C)(O)[C@H]1CC[C@H](NC(=O)c2cc(-c3cncs3)cc3c2ccn3COCC[Si](C)(C)C)CC1]

To a stirred solution of 6-(thiazol-5-yl)-1-((2-(trimethylsilyl)ethoxy)methyl)-1H-indole-4-carboxylic acid (0.20 g, 0.53 mmol) in DMF (2 mL), HATU (300 mg, 0.80 mmol) was added at 0° C. followed by DIPEA (0.28 mL, 1.60 mmol) under nitrogen atmosphere and the reaction mixture was stirred at 0° C. for 10 min. Then 2-((1r,4r)-4-aminocyclohexyl) propan-2-ol (100 mg, 0.64 mmol) was added and the reaction mixture was stirred for another 16 h at RT. The reaction mixture was diluted with ice cold water (10 mL) and extracted with EtOAc (2×10 mL). The resulting organic layer was washed with water (2×10 mL), brine solution (2×10 mL), dried over anhydrous Na₂SO₄, filtered and the filtrate was concentrated under vacuum. The resulting crude residue was used for next step without further purification. Yield: 87% (0.24 g, off white solid). LCMS: (Method C) 514.3 (M+H).

### Step 2: N-((1r,4r)-4-(2-hydroxypropan-2-yl)cyclohexyl)-6-(thiazol-5-yl)-1H-indole-4-carboxamide

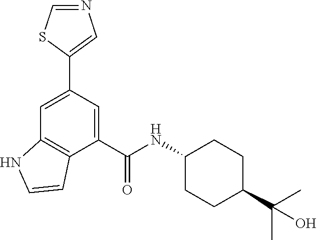 [SMILES: CC(C)(O)[C@H]1CC[C@H](NC(=O)c2cc(-c3cncs3)cc3[nH]ccc23)CC1]

To a stirred solution N-((1r,4r)-4-(2-hydroxypropan-2-yl)cyclohexyl)-6-(thiazol-5-yl)-1-((2-(trimethylsilyl)ethoxy)methyl)-1H-indole-4-carboxamide (230 mg, 0.45 mmol) in THF (10 mL), a solution of TBAF in THF (1.34 mL, 1.0 M, 1.34 mmol) was added slowly at RT and the reaction mixture was heated to reflux for 16 h. After completion, the reaction mixture was concentrated under reduced pressure. The resulting residue was suspended with ice cold water (10 mL) and extracted with EtOAc (2×10 mL). The resulting organic layer was washed with water (2×10 mL), brine solution (2×10 mL), dried over anhydrous Na₂SO₄, filtered and the filtrate was concentrated under vacuum. The crude material was purified by Prep-HPLC (Method A). The prep fraction was concentrated under reduced pressure, the residue was diluted with 10% MeOH in DCM (10 mL), washed with 10% aq. NaHCO₃ solution (4 mL) followed by brine solution and water. The organic layer was dried over anhydrous Na₂SO₄, filtered, concentrated under reduced pressure and lyophilized to get the title compound. Yield: 11% (37.4 mg, off white solid). ¹H NMR (400 MHz, DMSO-d₆): δ 11.39 (s, 1H), 9.06 (d, J=0.8 Hz, 1H), 8.32 (d, J=0.8 Hz, 1H), 8.15 (d, J=8.0 Hz, 1H), 7.76 (s, 1H), 7.67 (d, J=1.6 Hz, 1H), 7.50-7.49 (m, 1H), 6.82 (d, J=2.0 Hz, 1H), 4.06 (s, 1H), 3.81-3.74 (m, 1H), 1.97-1.95 (m, 2H), 1.87-1.84 (m, 2H), 1.36-1.26 (m, 2H), 1.20-1.00 (m, 9H). LCMS: (Method C) 384.2 (M+H)

### Example 30: N-((1r,3r)-3-(2-methoxyethoxy)cyclobutyl)-6-(thiazol-5-yl)-1H-indole-4-carboxamide

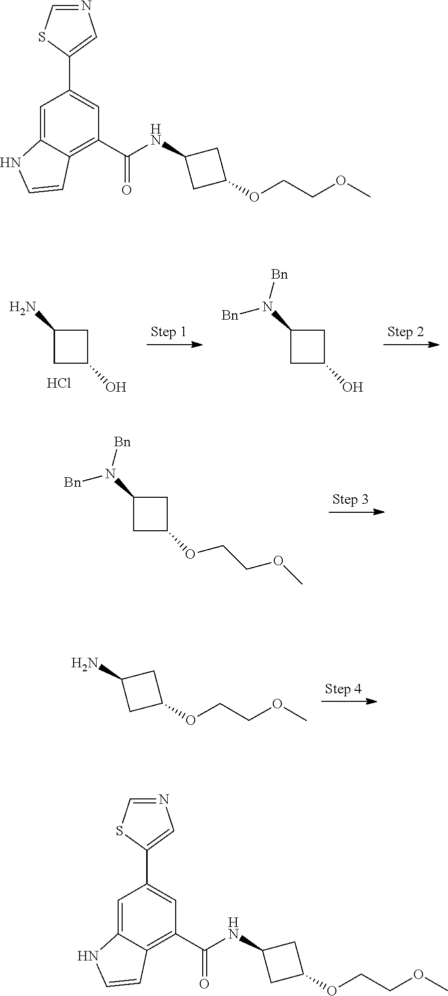

### Step 1: (1r,3r)-3-(dibenzylamino)cyclobutan-1-ol

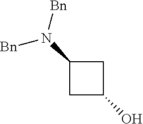 [SMILES: O[C@H]1C[C@H](N(Cc2ccccc2)Cc2ccccc2)C1]

To a stirred solution of trans-3-aminocyclobutanol hydrochloride (1.0 g, 8.09 mmol) in acetonitrile (20 mL), potassium carbonate (4.46 g, 3.23 mmol) and benzyl bromide (2.83 g, 16.59 mmol) were added at RT and the reaction mixture was heated at 75° C. for 16 h. The reaction mixture was filtered through Celite, the Celite bed was washed with EtOAc and the filtrate was concentrated under vacuum. The resulting crude residue was washed with petroleum ether to afford the title compound. Yield: 85% (1.85 g, white solid). ¹H NMR (400 MHz, DMSO-d₆): δ 7.33-7.20 (m, 10H), 4.84 (d, J=4.4 Hz, 1H), 4.17-4.14 (m, 1H), 3.44 (s, 4H), 3.36-3.30 (s, 1H), 2.13-2.07 (m, 2H), 1.87-1.81 (m, 2H). LCMS: (Method A) 268.2 (M+H)

### Step 2: (1r, 3r)-N, N-dibenzyl-3-(2-methoxyethoxy)cyclobutan-1-amine

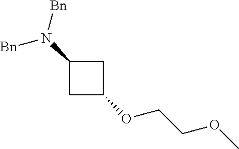 [SMILES: COCCO[C@H]1C[C@H](N(Cc2ccccc2)Cc2ccccc2)C1]

To a stirred solution of (1r,3r)-3-(dibenzylamino)cyclobutan-1-ol (1.8 g, 9.73 mmol) in DMPU (20 mL) at RT, sodium hydride (0.64 g, 16.10 mmol, 60% in paraffin oil) was added under nitrogen atmosphere. After stirring for 5 min, 1-bromo-2-methoxyethane (2.33 g, 16.80 mmol) was added at RT over a period of 10 min. During the addition of 1-bromo-2-methoxyethane frothing was observed. After complete addition, the reaction mixture was heated at 67° C. for 2 h. Additional sodium hydride (0.13 g, 3.25 mol, 60% in paraffin oil) followed by 1-bromo-2-methoxyethane (0.46 g, 3.30 mmol) were added at 67° C. and the reaction mixture was stirred at 67° C. for another 2 h. After completion (monitored by TLC), the reaction mixture was cooled to RT and slowly poured into ice (5 g) with continuous stirring. The suspension was extracted with EtOAc (3×30 mL). The combined organic layer was washed with water (2×25 mL), dried over anhydrous Na₂SO₄, filtered and the filtrate was concentrated under vacuum. The residue was dissolved in 1,4-dioxane (30 mL), to this HCl in 1,4-dioxane (4 N, 20 mL) was added and the reaction mixture was stirred for 20 min at RT. The mixture was concentrated under vacuum. The residual solid was dissolved in H₂O and basified with aq. NaOH solution (1 N) and extracted with EtOAc (3×30 mL). The combined organic layer was washed with water (2×25 mL), dried over anhydrous Na₂SO₄, filtered and the filtrate was concentrated under vacuum. Yield: 82% (1.8 g). ¹H NMR (400 MHz, CDCl₃): δ 7.32-7.29 (m, 8H), δ 7.25-7.22 (m, 2H), 4.09-4.04 (m, 1H), 3.57-3.49 (m, 8H), 3.48-3.40 (m, 4H), 2.16-2.13 (m, 4H). LCMS: (Method C) 326.1 (M+H)

### Step 3: (1r, 3r)-3-(2-methoxyethoxy)cyclobutan-1-amine

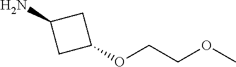

To a stirred solution of (1r,3r)-3-(dibenzylamino)cyclobutan-1-ol (1.8 g, 5.53 mmol) in absolute ethanol (20 mL), Pd(OH)₂/C (0.38 g, 20%) was added at RT and the mixture was stirred overnight under hydrogen atmosphere at RT. After completion (monitored by LCMS), the reaction mixture was filtered through Celite bed. The filtrate was concentrated under vacuum to afford the title compound which was used in the next step without further purification. Yield: 94% (0.78 g, colorless liquid). ¹H NMR (400 MHz, DMSO-d₆): 4.09-4.04 (m, 1H), 3.43-3.40 (m, 2H), 3.39-3.34 (m, 6H), 2.08-2.02 (m, 2H), 1.85-1.80 (m, 2H).

### Step 4: N-((1r,3r)-3-(2-methoxyethoxy)cyclobutyl)-6-(thiazol-5-yl)-1H-indole-4-carboxamide

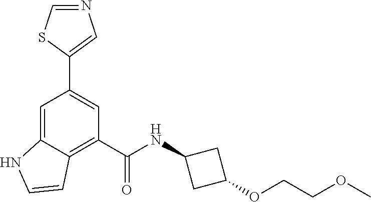 [SMILES: COCCO[C@H]1C[C@H](NC(=O)c2cc(-c3cncs3)cc3[nH]ccc23)C1]

To a stirred solution of 6-(thiazol-5-yl)-1H-indole-4-carboxylic acid (80 mg, 0.32 mmol) in DMF (3 mL) at RT, were added HATU (212 mg, 0.55 mmol) and DIPEA (127 mg, 0.98 mmol) followed by (1r,3r)-3-(2-methoxyethoxy)cyclobutan-1-amine (57 mg, 0.39 mmol) and the reaction mixture was stirred at RT for 16 h. After completion (monitored by TLC), the reaction mixture was quenched with water (10 mL) and extracted with EtOAc (3×20 mL). The combined organic layer was washed with water (10 mL), brine (10 mL), dried over anhydrous Na₂SO₄ and concentrated under vacuum. The resulting crude residue was purified by flash chromatography on Biotage Isolera (200-400 mesh silica gel, eluent: 0-5% methanol in DCM) to afford the title compound. Yield: 17% (21 mg, off white solid). ¹H NMR (400 MHz, DMSO-d₆): δ 11.41 (s, 1H), 9.06 (d, J=0.8 Hz, 1H), 8.63 (d, J=7.2 Hz, 1H), 8.32 (d, J=0.8 Hz, 1H), 7.77 (s, 1H), 7.71 (d, J=1.2 Hz, 1H), 7.52-7.50 (m, 1H), 6.84-6.83 (m, 1H), 4.53-4.48 (m, 1H), 4.18-4.15 (m, 1H), 3.47-3.43 (m, 4H), 3.33 (s, 3H), 2.35-2.27 (m, 4H). LCMS: (Method C) 372.1 (M+H)

### Example 31: N-((1R,3R)-3-hydroxycyclopentyl)-6-(thiazol-5-yl)-1H-indole-4-carboxamide

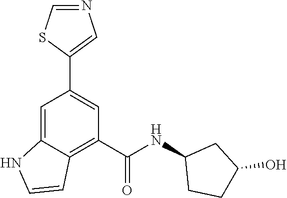 [SMILES: O=C(N[C@@H]1CC[C@@H](O)C1)c1cc(-c2cncs2)cc2[nH]ccc12]

To a stirred solution of 6-(thiazol-5-yl)-1H-indole-4-carboxylic acid (80 mg, 0.32 mmol) in DMF (5 mL), were added HATU (212 mg, 0.55 mmol) and DIPEA (147 mg, 1.14 mmol) followed by (trans)-3-aminocyclopentan-1-ol (54 mg, 0.39 mmol) and the reaction mixture was stirred at RT for 16 h. After completion (monitored by TLC), the reaction mixture was quenched with water (10 mL) and extracted with EtOAc (3×20 mL). The combined organic layer was washed with water (10 mL), brine (10 mL), dried over anhydrous Na₂SO₄ and concentrated under vacuum. The resulting crude residue was purified by flash chromatography on Biotage Isolera (200-400 mesh silica gel, eluent: 0-5% methanol in DCM) to afford the title compound Yield: 34% (36.67 mg, off white solid). ¹H NMR (400 MHz, DMSO-d₆): δ 11.39 (s, 1H), 9.06 (d, J=0.8 Hz, 1H), 8.31 (d, J=0.4 Hz, 1H), 8.26 (d, J=7.6 Hz, 1H), 7.76-7.75 (m, 1H), 7.67 (d, J=1.6 Hz, 1H), 7.51-7.49 (m, 1H), 6.82-6.81 (m, 1H), 4.54-4.49 (m, 2H), 4.26-4.22 (m, 1H), 2.13-2.08 (m, 1H), 1.96-1.87 (m, 2H), 1.78-1.71 (m, 1H), 1.57-1.48 (m, 2H). LCMS: (Method C) 328.0 (M+H)

### Example 32: N-((1s,3s)-3-(2-methoxyethoxy)cyclobutyl)-6-(thiazol-5-yl)-1H-indole-4-carboxamide

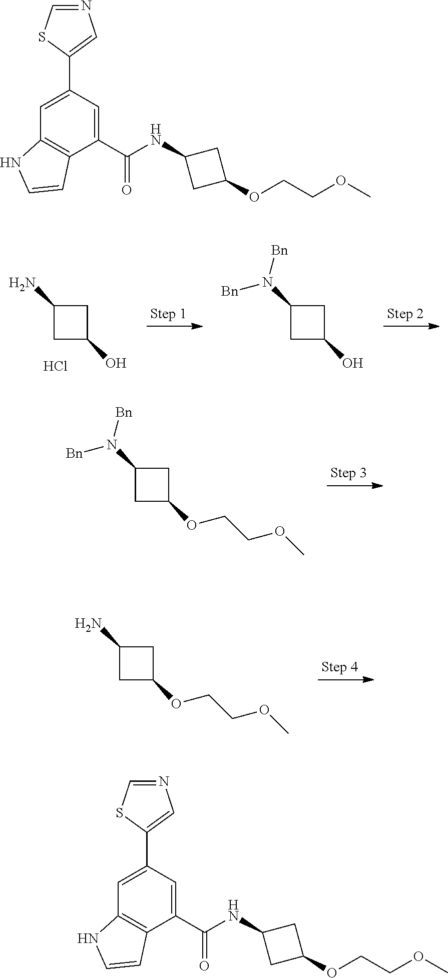

### Step 1: (1s,3s)-3-(dibenzylamino) cyclobutan-1-ol

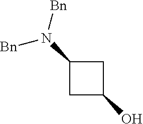 [SMILES: O[C@H]1C[C@@H](N(Cc2ccccc2)Cc2ccccc2)C1]

To a stirred solution of cis-3-aminocyclobutanol hydrochloride (1.0 g, 8.09 mmol) in acetonitrile (20 mL), potassium carbonate (4.46 g, 32.27 mmol) and benzyl bromide (2.83 g, 16.59 mmol) were added at RT and the reaction mixture was heated at 75° C. for 16 h. The solid residue was filtered washed with EtOAc and the filtrate was concentrated under vacuum. The resulting solid residue was washed with petroleum ether to afford the title compound. Yield: 87% (1.9 g, white solid). ¹H NMR (400 MHz, CDCl₃): δ 7.35-7.24 (m, 10H), 4.01-3.92 (m, 1H), 3.51 (s, 4H), 2.72-2.67 (m, 1H), 2.50-2.43 (m, 2H), 1.84-1.76 (m, 2H). LCMS: (Method C) 268.1 (M+H)

### Step 2: (1s, 3s)-N,N-dibenzyl-3-(2-methoxyethoxy)cyclobutan-1-amine

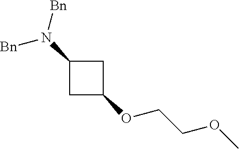 [SMILES: COCCO[C@H]1C[C@@H](N(Cc2ccccc2)Cc2ccccc2)C1]

To a stirred solution of cis-3-(dibenzylamino)cyclobutan-1-ol (1.9 g, 7.11 mmol) in DMPU (10 mL), sodium hydride (0.68 g, 17.0 mmol, 60% in paraffin oil) was added at RT under nitrogen atmosphere. After 5 min of stirring at RT, 1-bromo-2-methoxyethane (2.46 g, 17.7 mmol) was added over a period of 10 min at RT. During the addition of 1-bromo-2-methoxyethane foam formation was observed. Then the reaction mixture was stirred for 4 h at 65° C. After completion, the reaction mixture was cooled to RT and slowly poured into ice cold water with continuous stirring. The suspension was extracted with EtOAc (3×30 mL). The combined organic layer was washed with water (2×25 mL), dried over anhydrous Na₂SO₄, filtered and the filtrate was concentrated under vacuum. The resulting crude residue was dissolved in 1,4-dioxane (30 mL), HCl in 1,4-dioxane (20 mL, 4 N) was added and the mixture was stirred for 20 min at RT. The reaction mixture was concentrated under vacuum and the resulting solid was dissolved in H₂O and basified with aq. NaOH solution (1 N). The suspension was extracted with EtOAc (3×30 mL). The combined organic layer was washed with water (2×25 mL), dried over anhydrous Na₂SO₄, filtered and the filtrate was concentrated under vacuum. Yield: 82% (1.9 g, colorless liquid). ¹H NMR (400 MHz, DMSO-d₆): δ 7.34-7.23 (m, 10H), 3.59-3.56 (m, 1H), 3.46 (s, 4H), 3.39-3.33 (m, 4H), 3.22-3.16 (m, 4H), 2.31-2.27 (m, 2H), 1.66-1.59 (m, 2H). LCMS: (Method C) 326.2 (M+H)

### Step 3: (1s,3s)-3-(2-methoxyethoxy)cyclobutan-1-amine

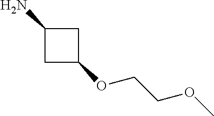

To a stirred solution of cis-N,N-dibenzyl-3-(2-methoxyethoxy)cyclobutan-1-amine (1.9 g, 5.84 mmol) in absolute ethanol (20 mL), Pd(OH)₂/C (0.4 g, 20%) was added at RT and the mixture was stirred for overnight under hydrogen at RT. After completion of the reaction, the reaction mixture was filtered through Celite bed. The filtrate was evaporated under vacuum to afford the title compound. It was used in the next step without further purification. Yield: 96% (0.81 g, colorless liquid). LCMS: (Method C) 146.2 (M+H)

### Step 4: N-((1s,3s)-3-(2-methoxyethoxy)cyclobutyl)-6-(thiazol-5-yl)-1H-indole-4-carboxamide

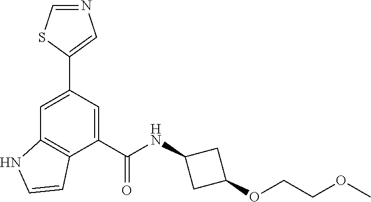 [SMILES: COCCO[C@H]1C[C@@H](NC(=O)c2cc(-c3cncs3)cc3[nH]ccc23)C1]

To a stirred solution of 6-(thiazol-5-yl)-1H-indole-4-carboxylic acid (0.1 g, 0.41 mmol) in DMF (2 mL) at 0° C. were added DIPEA (0.22 mL, 1.27 mmol) and HATU (0.233 g, 0.61 mmol). After stirring for 5 min, a solution of cis-3-(2-methoxyethoxy) cyclobutan-1-amine (89 mg, 0.61 mmol) in DMF (2.5 mL) was added and the reaction mixture was stirred at RT for 2.5 h. The reaction mixture was monitored by TLC. After completion, the reaction mixture was diluted with water (5 mL) and the resulting suspension was extracted with EtOAc (2×10 mL). The combined organic layer was washed with water (20 mL), brine (20 mL), dried over anhydrous Na₂SO₄ and concentrated under vacuum. The crude residue was purified by Prep-HPLC (Method A). The prep. fraction was concentrated under reduced pressure. The residue was diluted with 10% MeOH in DCM (10 mL) and washed with 10% aq. NaHCO₃ solution (4 mL) followed by brine solution and water. The organic layer was dried over anhydrous Na₂SO₄, filtered, dried under reduced pressure and lyophilized to get the title compound. Yield: 61% (40.53 mg, yellow gummy solid). ¹H NMR (400 MHz, DMSO-d₆): δ 11.39 (s, 1H), 9.06 (s, 1H), 8.58 (d, J=8.0 Hz, 1H), 8.33 (s, 1H), 7.77 (s, 1H), 7.75 (s, 1H), 7.51-7.49 (m, 1H), 6.86 (s, 1H), 4.16-4.08 (m, 1H), 3.78-3.71 (m, 1H), 3.44 (s, 4H), 3.26 (s, 3H), 2.68-2.62 (m, 2H), 2.06-1.99 (m, 2H). LCMS: (Method C) 372.1 (M+H)

### Example 33: N-(4-morpholinobenzyl)-6-(thiazol-5-yl)-1H-indole-4-carboxamide

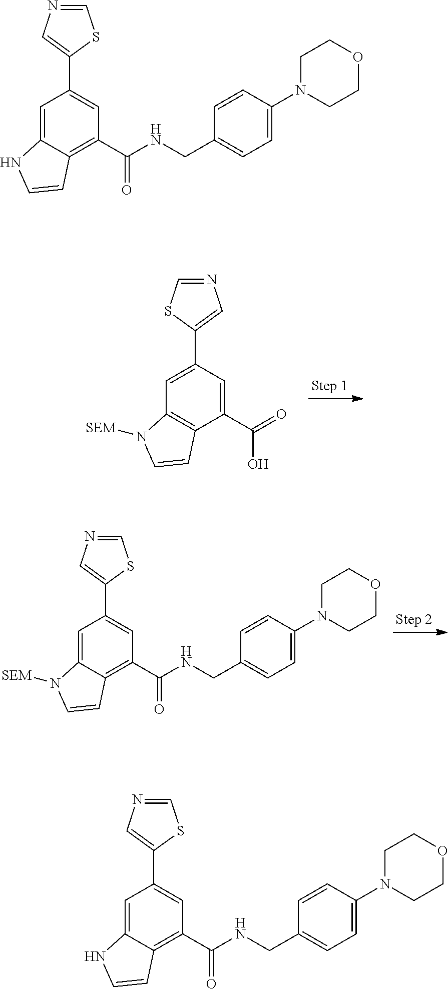 [SMILES: O=C(NCc1ccc(N2CCOCC2)cc1)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)NCc3ccc(N4CCOCC4)cc3)cc(-c3cncs3)cc21 ; O=C(NCc1ccc(N2CCOCC2)cc1)c1cc(-c2cncs2)cc2[nH]ccc12]

### Step 1: N-(4-morpholinobenzyl)-6-(thiazol-5-yl)-1-((2-(trimethylsilyl)ethoxy)methyl)-1H-indole-4-carboxamide

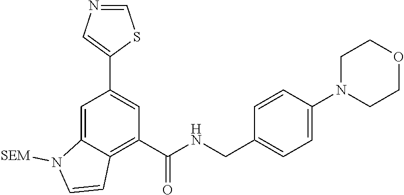 [SMILES: C[Si](C)(C)CCOCn1ccc2c(C(=O)NCc3ccc(N4CCOCC4)cc3)cc(-c3cncs3)cc21]

To a stirred solution of 6-(thiazol-5-yl)-1-((2-(trimethylsilyl)ethoxy)methyl)-1H-indole-4-carboxylic acid (200 mg, 0.53 mmol) in DMF (5 mL) HATU (304 mg, 0.80 mmol) and DIPEA (0.24 mL, 1.33 mmol) were added under nitrogen atmosphere at 0° C. After 10 min (4-morpholinophenyl)methanamine (123 mg, 0.64 mmol) was added and stirred at RT for 16 h. The reaction mixture was diluted with ice cold water (10 mL) and extracted with EtOAc (50 mL). The organic solution was washed with brine (2×50 mL), water (2×25 mL), dried over anhydrous Na₂SO₄, and the filtrate was concentrated under vacuum. The resulting crude product was used for next step without further purification. Yield: 75% (220 mg, Brown gummy solid). LCMS: (Method C) 549.3 (M+H)

### Step 2 N-(4-morpholinobenzyl)-6-(thiazol-5-yl)-1H-indole-4-carboxamide

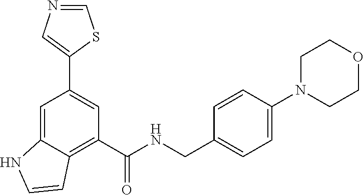 [SMILES: O=C(NCc1ccc(N2CCOCC2)cc1)c1cc(-c2cncs2)cc2[nH]ccc12]

To a stirred solution of N-(4-morpholinobenzyl)-6-(thiazol-5-yl)-1-((2-(trimethylsilyl)ethoxy)methyl)-1H-indole-4-carboxamide (200 mg, 0.36 mmol) in THF (5 mL) TBAF (1.0 M in THF) (1.09 mL, 1.09 mmol) was added at RT and stirred for 16 h at 70° C. The reaction mixture was quenched with ice cold water (10 mL) and then extracted with EtOAc (50 mL). The organic solution was washed with water (2×50 mL), brine (2×50 mL), dried over anhydrous Na₂SO₄, filtered and the filtrate was concentrated under vacuum. The resulting crude was purified by Prep-HPLC (Method A). The prep-fraction was collected, concentrated under reduced pressure, dissolved in 10% MeOH in DCM (10 mL) and washed with water. The organic layer was dried over anhydrous Na₂SO₄, concentrated under reduced pressure and lyophilized to get the title compound. Yield: 4% (5.53 mg, Pale brown solid). ¹HNMR (400 MHz, DMSO-d₆): 11.43 (s, 1H), 9.05 (s, 1H), 8.87 (t, J=6.0 Hz, 1H), 8.31 (s, 1H), 7.79 (s, 2H), 7.5 (t, J=2.8 Hz, 1H), 7.21 (s, 1H), 7.24 (s, 1H), 6.94-6.92 (m, 3H), 4.50 (d, J=5.6 Hz, 2H), 3.7 (t, J=4.4 Hz, 4H), 3.1 (t, J=4.8 Hz, 4H). LCMS: (Method C) 419.2 (M+H)

### Example 34: N-(Trans-3-(2-methoxyethoxy)cyclopentyl)-6-(thiazol-5-yl)-1H-indole-4-carboxamide

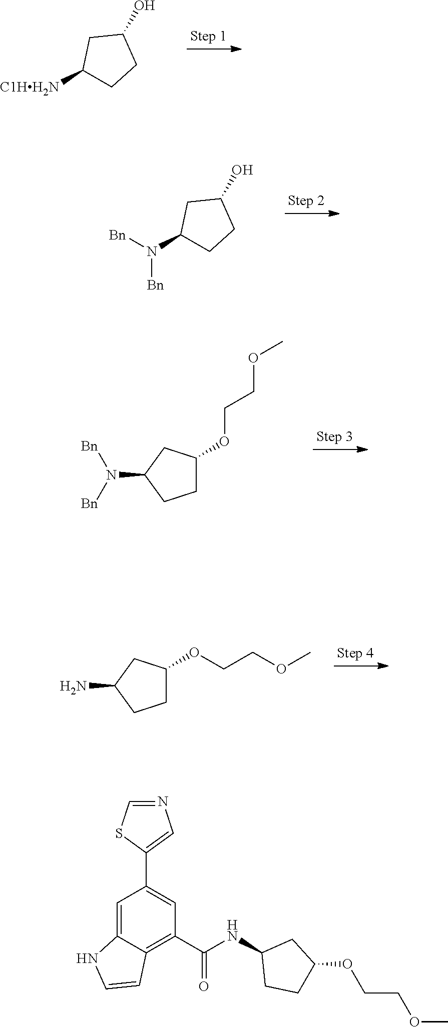

### Step 1: Trans-3-(dibenzylamino)cyclopentan-1-ol

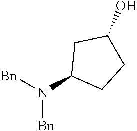 [SMILES: O[C@@H]1CC[C@@H](N(Cc2ccccc2)Cc2ccccc2)C1]

To a stirred solution of trans-3-aminocyclopentan-1-ol hydrochloride¹ (0.80 g, 5.81 mmol) in CH₃CN (10 mL), potassium carbonate (2.40 g, 17.44 mmol) and benzyl bromide (2.03 g, 11.91 mmol) were added at RT and the reaction mixture was heated at 75° C. for 5 h. After completion (monitored by LCMS), the reaction mixture was quenched with ice cold water (30 mL). The solid precipitate was collected by filtration and washed with cold water (3×10 mL). The solid residue was dissolved in EtOAc (50 mL), dried over anhydrous Na₂SO₄, filtered and the filtrate was concentrated under vacuum. The resulting crude residue was washed with cold petroleum ether to afford the title compound. Yield: 49% (0.80 g, colorless liquid). ¹H NMR (400 MHz, CDCl₃): δ 7.39-7.31 (m, 4H), 7.31-7.24 (m, 4H), 7.23-7.21 (m, 2H), 4.41-4.37 (m, 1H), 3.61 (s, 4H), 3.57-3.51 (m, 1H), 2.02-1.94 (m, 2H), 1.85-1.79 (m, 2H), 1.66-1.56 (m, 2H). LCMS: (Method C) 282.2 (M+H) ¹ Although trans-3-aminocyclopentan-1-ol hydrochloride is structurally depicted as a single enantiomer, the structure is meant to depict relative and not absolute stereochemistry, i.e., the compound is a racemic mixture of the two trans stereoisomers.

### Step 2: Trans-N,N-dibenzyl-3-(2-methoxyethoxy)cyclopentan-1-amine

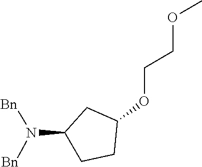 [SMILES: COCCO[C@@H]1CC[C@@H](N(Cc2ccccc2)Cc2ccccc2)C1]

To a stirred solution of trans-3-(dibenzylamino)cyclopentan-1-ol (0.8 g, 2.84 mmol) in DMPU (10 mL), sodium hydride (257 mg, 6.96 mmol, 60% in paraffin oil) was added at RT under nitrogen atmosphere. After stirring for 5 min, 1-bromo-2-methoxyethane (987 mg, 7.10 mmol) was added at RT over a period of 10 min. During the addition of 1-bromo-2-methoxyethane foaming was observed. After complete addition, the reaction mixture was stirred for 1 h at 65° C. An additional portion of sodium hydride (257 mg, 6.96 mmol, 60% in paraffin oil) followed by 1-bromo-2-methoxyethane (987 mg, 7.10 mmol) were added at 65° C. The reaction mixture was stirred at 65° C. for another 2 h. After completion (monitored by TLC), the reaction mixture was cooled to RT and slowly poured into ice with continuous stirring. The suspension was extracted with EtOAc (3×30 mL). The combined organic layer was washed with water (2×15 mL), dried over anhydrous Na₂SO₄, filtered and the filtrate was concentrated under vacuum. The resulting crude residue was purified by flash chromatography on Biotage Isolera (silica gel: 230-400 mesh, eluent: 10-40% EtOAc in petroleum ether) to get the title compound. Yield: 83% (0.80 g, light yellow gum). ¹H NMR (400 MHz, DMSO-d₆): δ 7.35-7.28 (m, 8H), 7.23-7.19 (m, 2H), 3.91-3.85 (m, 1H), 3.53 (s, 4H), 3.38-3.25 (m, 5H), 3.19 (s, 3H), 1.88-1.77 (m, 1H), 1.76-1.62 (m, 3H), 1.52-1.44 (m, 2H). LCMS: (Method C) 340.3 (M+H)

### Step 3: Trans-3-(2-methoxyethoxy)cyclopentan-1-amine

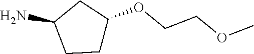

To a stirred solution of trans-N,N-dibenzyl-3-(2-methoxyethoxy)cyclopentan-1-amine (0.80 g, 2.36 mmol) in absolute ethanol (20 mL), Pd(OH)₂/C (150 mg, 20%) was added at RT and the mixture was stirred overnight under hydrogen atmosphere at RT. After completion (monitored by LCMS), the reaction mixture was filtered through Celite bed. The filtrate was evaporated under vacuum to afford the title compound which was used in the next step without further purification. Yield: 93% (350 mg, colorless liquid). ¹H NMR (400 MHz, DMSO-d₆): δ 3.94-3.92 (m, 1H), 3.28-3.23 (m, 3H), 3.23-3.22 (m, 2H), 1.95-1.72 (m, 3H), 1.58-1.41 (m, 2H), 1.39-1.18 (m, 1H). LCMS: (Method C) 160.2 (M+H)

### Step 4: N-(Trans-3-(2-methoxyethoxy)cyclopentyl)-6-(thiazol-5-yl)-1H-indole-4-carboxamide

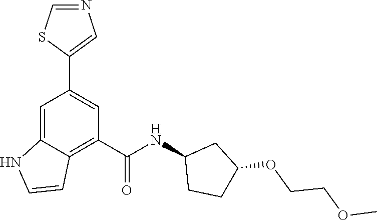 [SMILES: COCCO[C@@H]1CC[C@@H](NC(=O)c2cc(-c3cncs3)cc3[nH]ccc23)C1]

To a stirred solution of 6-(thiazol-5-yl)-1H-indole-4-carboxylic acid (80 mg, 0.32 mmol) in DMF (10 mL) were added EDCI (125 mg, 0.65 mmol), HOBt (100 mg, 0.65 mmol) and DIPEA (126 mg, 0.98 mmol) at RT. After stirring for 5 min, trans-3-(2-methoxyethoxy)cyclopentan-1-amine (104 mg, 0.65 mmol) was added at RT and the reaction mixture was stirred at RT for 16 h. The reaction mixture was monitored by TLC and quenched with water (15 mL). The resulting suspension was extracted with EtOAc (3×15 mL). The combined organic layer was washed with water (10 mL), brine (10 mL), dried over anhydrous Na₂SO₄ and solvent was evaporated under vacuum. The resulting crude residue was purified by flash chromatography on Biotage Isolera (230-400 mesh silica gel, eluent with 0-5% methanol in DCM) to afford the title compound. Yield: 28% (35 mg, yellow gummy solid). ¹H NMR (400 MHz, DMSO-d₆): δ 11.40 (s, 1H), 9.06 (d, J=0.8 Hz, 1H), 8.31-8.29 (m, 2H), 7.8 (s, 1H), 7.7 (d, J=1.6 Hz, 1H), 7.5 (t, J=2.8 Hz, 1H), 6.8 (t, J=2.0 Hz, 1H), 4.45-4.41 (m, 1H), 4.04-4.01 (m, 1H), 3.48-3.44 (m, 4H), 3.3 (s, 3H), 2.06-1.81 (m, 3H), 1.79-1.74 (m, 1H), 1.64-1.57 (m, 2H). LCMS: (Method A) 386.1 (M+H)

### Example 35: N-(piperidin-4-yl)-6-(thiazol-5-yl)-1H-indole-4-carboxamide hydrochloride

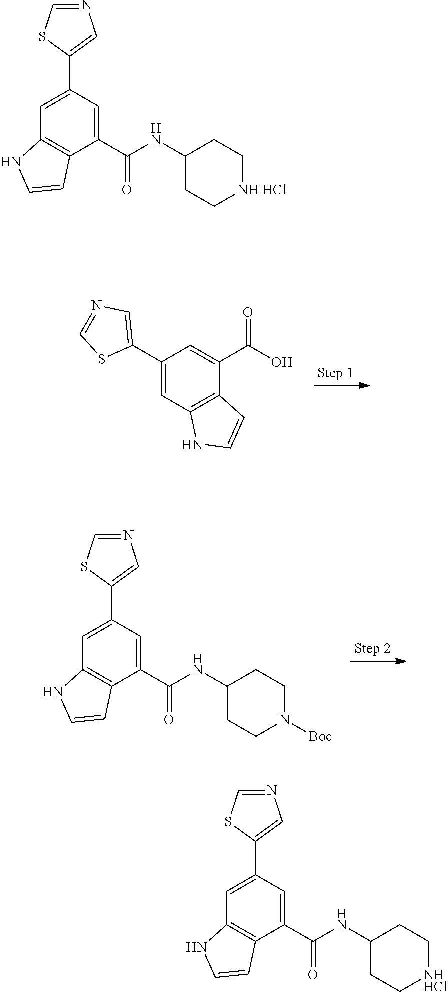

### Step 1: tert-butyl 4-(6-(thiazol-5-yl)-1H-indole-4-carboxamido)piperidine-1-carboxylate

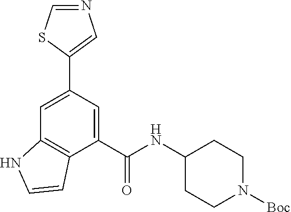 [SMILES: CC(C)(C)OC(=O)N1CCC(NC(=O)c2cc(-c3cncs3)cc3[nH]ccc23)CC1]

To a stirred solution of 6-(thiazol-5-yl)-1H-indole-4-carboxylic acid (150 mg, 0.61 mmol) in DMF (3 mL) at 0° C., HATU (280 mg, 0.74 mmol) was added followed by DIPEA (0.237 g, 1.84 mmol) under nitrogen atmosphere and the stirring was continued at 0° C. for 10 min. Then tert-butyl 4-aminopiperidine-1-carboxylate (147 mg, 0.73 mmol) was added and the reaction mixture was stirred for another 16 h at RT. The reaction mixture was diluted with ice cold water (10 mL) and extracted with EtOAc (2×10 mL). The combined organic layer was washed with water (2×10 mL), brine solution (2×10 mL), dried over anhydrous Na₂SO₄, filtered and the filtrate was concentrated under vacuum. The resulting crude residue was purified by flash chromatography on Biotage Isolera (230-400 mesh silica gel, eluent: 70% EtOAc in petroleum ether) to afford the title compound. Yield: 50% (0.13 g). ¹H NMR (400 MHz, DMSO-d₆): δ 11.41 (s, 1H), 9.06 (s, 1H), 8.31 (s, 1H), 8.26 (d, J=8.0 Hz, 1H), 7.77 (s, 1H), 7.69 (d, J=1.2 Hz, 1H), 7.52-7.50 (m, 1H), 6.83 (s, 1H), 4.07-4.06 (m, 3H), 2.88 (br s, 2H), 1.90-1.83 (m, 2H), 1.49-1.39 (s, 11H). LCMS: (Method B) 425.1 (M−H)

### Step 2: N-(piperidin-4-yl)-6-(thiazol-5-yl)-1H-indole-4-carboxamide hydrochloride

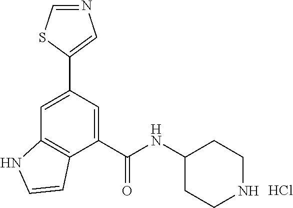 [SMILES: O=C(NC1CCNCC1)c1cc(-c2cncs2)cc2[nH]ccc12 ; Cl]

To a stirred solution tert-butyl 4-(6-(thiazol-5-yl)-1H-indole-4-carboxamido)piperidine-1-carboxylate (105 mg, 0.25 mmol) in 1,4-dioxane (5 mL) at RT was added HCl in 1,4-dioxane (5 mL, 4 M) slowly and the reaction mixture was stirred at RT for 2 h. After completion, the reaction mixture was concentrated under reduced pressure. The resulting crude residue was triturated with diethyl ether (2×10 mL) to afford the title compound. Yield: 11% (13.3 mg, white solid). ¹H NMR (400 MHz, DMSO-d₆): δ 11.49 (s, 1H), 9.09 (m, 1H), 8.98 (br s, 1H), 8.88-8.82 (m, 1H), 8.53 (d, J=7.6 Hz, 1H), 8.35 (d, J=0.8 Hz, 1H), 7.79-7.78 (m, 1H), 7.74 (d, J=1.6 Hz, 1H), 7.52-7.51 (m, 1H), 6.83-6.82 (m, 1H), 4.17-4.15 (m, 1H), 3.39-3.35 (m, 2H), 3.07-3.07 (m, 2H), 2.06-2.06 (m, 2H), 1.88-1.87 (m, 2H). LCMS: (Method B) 327.2 (M+H)

### Example 36: N-(3-hydroxy-3-methylcyclobutyl)-6-(thiazol-5-yl)-1H-indole-4-carboxamide

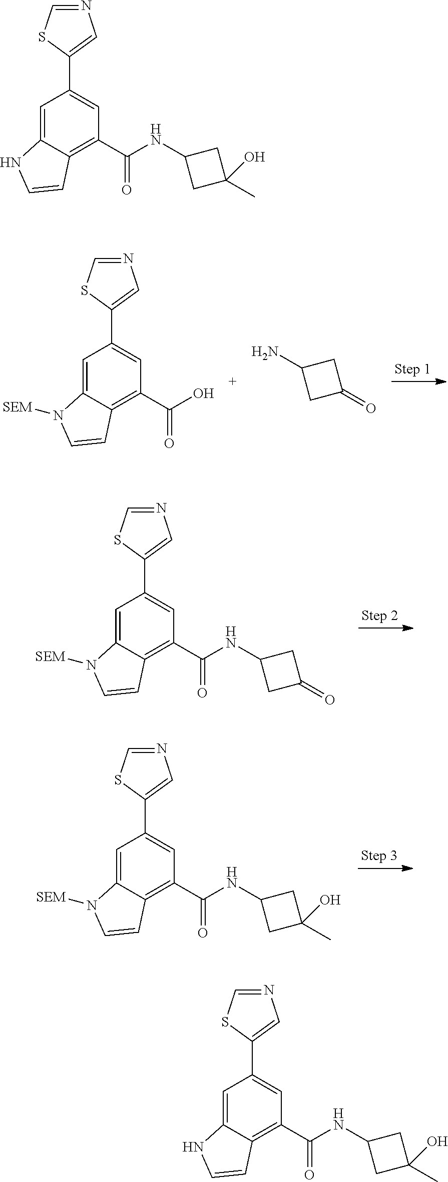 [SMILES: CC1(O)CC(NC(=O)c2cc(-c3cncs3)cc3[nH]ccc23)C1 ; C[Si](C)(C)CCOCn1ccc2c(C(=O)O)cc(-c3cncs3)cc21 ; NC1CC(=O)C1 ; C[Si](C)(C)CCOCn1ccc2c(C(=O)NC3CC(=O)C3)cc(-c3cncs3)cc21 ; CC1(O)CC(NC(=O)c2cc(-c3cncs3)cc3c2ccn3COCC[Si](C)(C)C)C1 ; CC1(O)CC(NC(=O)c2cc(-c3cncs3)cc3[nH]ccc23)C1]

### Step 1: N-(3-oxocyclobutyl)-6-(thiazol-5-yl)-1-((2-(trimethylsilyl)ethoxy)methyl)-1H-indole-4-carboxamide

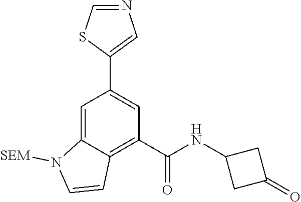 [SMILES: C[Si](C)(C)CCOCn1ccc2c(C(=O)NC3CC(=O)C3)cc(-c3cncs3)cc21]

To a stirred solution of 6-(thiazol-5-yl)-1-((2-(trimethylsilyl)ethoxy)methyl)-1H-indole-4-carboxylic acid (0.30 g, 0.80 mmol) in DMF (5 mL) at RT, were added HATU (517 mg, 1.36 mmol) and DIPEA (0.41 mL, 310 mg, 2.40 mmol) followed by 3-aminocyclobutan-1-one (143 mg, 0.96 mmol) and the reaction mixture was stirred at RT for 16 h. After completion (monitored by TLC), the reaction mixture was quenched with water (10 mL) and extracted with EtOAc (3×20 mL). The combined organic layer was washed with water (10 mL), brine (10 mL), dried over anhydrous Na₂SO₄ and concentrated under vacuum. The resulting crude residue was purified by flash chromatography on Biotage Isolera (200-400 mesh silica gel, eluent: 0-5% methanol in DCM) to afford the title compound. Yield: 71% (250 mg, white solid). ¹H NMR (400 MHz, DMSO-d₆): δ 9.09 (d, J=0.4 Hz, 1H), 8.93 (d, J=6.8 Hz, 1H), 8.36-8.35 (m, 1H), 8.03 (s, 1H), 7.84-7.82 (m, 1H), 7.65 (d, J=3.2 Hz, 1H), 6.95-6.92 (m, 1H), 5.67 (s, 2H), 4.67-4.61 (m, 1H), 3.49-3.41 (m, 4H), 3.33-3.30 (m, 2H), 0.8 (t, J=8.0 Hz, 2H), −0.10 (s, 9H). LCMS: (Method A) 442.0 (M+H)

### Step 2: N-(3-hydroxy-3-methylcyclobutyl)-6-(thiazol-5-yl)-1-((2-(trimethylsilyl)ethoxy)methyl)-1H-indole-4-carboxamide

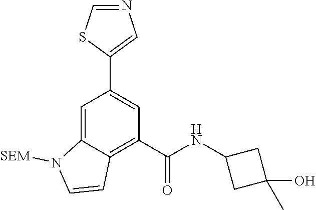 [SMILES: CC1(O)CC(NC(=O)c2cc(-c3cncs3)cc3c2ccn3COCC[Si](C)(C)C)C1]

To a stirred solution of N-(3-oxocyclobutyl)-6-(thiazol-5-yl)-1-((2-(trimethylsilyl)ethoxy) methyl)-1H-indole-4-carboxamide (250 mg, 0.56 mmol) in a mixture of THF (10 mL) and diethyl ether (10 mL), was added MeLi (0.78 mL, 1.6 M in diethyl ether, 1.24 mmol) at 0° C. and the reaction mixture was stirred for 20 min at RT. The reaction mixture was quenched with methanol and concentrated under reduced pressure. The resulting crude residue was purified by flash chromatography on Biotage Isolera (silica gel: 100-200 mesh, eluent: 1-5% MeOH in DCM) to get the title compound. Yield: 58% (150 mg, yellow solid). LCMS: (Method A) 458.1 (M+H)

### Step 3: N-(3-hydroxy-3-methylcyclobutyl)-6-(thiazol-5-yl)-1H-indole-4-carboxamide

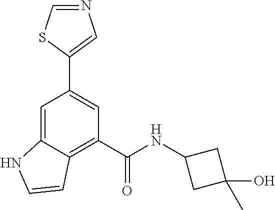 [SMILES: CC1(O)CC(NC(=O)c2cc(-c3cncs3)cc3[nH]ccc23)C1]

To a stirred solution of N-(3-hydroxy-3-methylcyclobutyl)-6-(thiazol-5-yl)-1-((2-(trimethylsilyl)ethoxy)methyl)-1H-indole-4-carboxamide (150 mg, 0.56 mmol) in THF (10 mL), was added TBAF (0.99 mL, 1.0 M in THF, 0.99 mmol) at RT and the reaction mixture was stirred for 16 h at 75° C. After completion (monitored by TLC), the reaction mixture was quenched with water (10 mL) and extracted with EtOAc (3×20 mL). The combined organic layer was washed with water (10 mL), brine (10 mL) and dried over anhydrous Na₂SO₄. The solvent was evaporated in vacuum. The resulting crude residue was purified by flash chromatography on Biotage Isolera (200-400 mesh silica gel, eluent 0-5% methanol in DCM) followed by Prep-HPLC (Method A) to afford the title compound. Yield: 2% (2.1 mg, white solid). ¹H NMR (400 MHz, DMSO-d₆): δ 11.39 (s, 1H), 9.06 (d, J=0.4 Hz, 1H), 8.54 (d, J=7.2 Hz, 1H), 8.33-8.32 (m, 1H), 7.76 (s, 1H), 7.74 (d, J=1.6 Hz, 1H), 7.51-7.49 (m, 1H), 6.86-6.85 (m, 1H), 4.99 (s, 1H), 4.09-4.03 (m, 1H), 2.36-2.32 (m, 2H), 2.18-2.13 (m, 2H), 1.30 (s, 3H). LCMS: (Method A) 328.2 (M+H)

### Example 37: N-((1r,4r)-4-(3-methoxypropoxy)cyclohexyl)-6-(thiazol-5-yl)-1H-indole-4-carboxamide

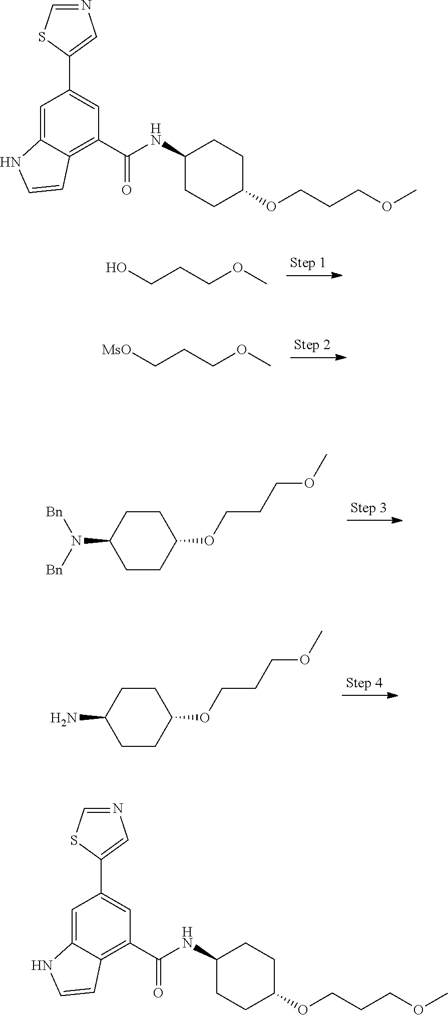

### Step 1: 3-methoxypropyl methanesulfonate

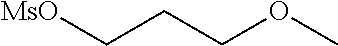

To a stirred solution of 3-methoxypropanol (1.00 g, 11.11 mmol) and triethylamine (4.6 mL, 33.33 mmol) in DCM (20 mL) at 0° C. was added methanesulfonyl chloride (1.52 g, 13.33 mmol) slowly and the reaction mixture was stirred at RT for 1 h. After completion (monitored by TLC), the reaction mixture was quenched with sat. aq. NaHCO₃ and extracted with EtOAc. The combined organic layer was washed with brine, dried over anhydrous Na₂SO₄, filtered and concentrated under reduced pressure to get the title compound which was used in the next step without further purification. Yield: 80% (1.5 g, colorless liquid). ¹H NMR (400 MHz, DMSO-d₆): δ 4.26-4.23 (m, 2H), 3.42-3.39 (m, 2H), 3.26 (s, 3H), 3.21 S, 3H), 1.93-1.87 (m, 2H).

### Step 2: (1r,4r)-N,N-dibenzyl-4-(3-methoxypropoxy)cyclohexan-1-amine

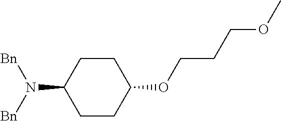 [SMILES: COCCCO[C@H]1CC[C@H](N(Cc2ccccc2)Cc2ccccc2)CC1]

To a stirred solution of (1r,4r)-4-(dibenzylamino)cyclohexan-1-ol (1.0 g, 3.39 mmol) in DMPU (20 mL), sodium hydride (333 mg, 8.47 mmol, 60% in paraffin oil) was added at RT under nitrogen atmosphere. After stirring for 5 min (the mixture became exothermic), a solution of 3-methoxypropyl methanesulfonate (1.42 g, 8.47 mmol) in DMPU (2 mL) was added over a period of 10 min. The reaction mixture was then stirred for 3 h at 65° C. After completion (TLC), the reaction mixture was cooled to RT and slowly poured into ice-water (50 mL) with continuous stirring. The suspension was extracted with EtOAc (3×100 mL). The combined organic layer was washed with water (2×100 mL), dried over anhydrous Na₂SO₄, filtered and the filtrate was concentrated under vacuum. The resulting crude residue was purified by flash chromatography on Biotage Isolera (silica gel: 230-400 mesh, eluent: 10-40% EtOAc in petroleum ether) to get the title compound. Yield: 62% (750 mg). ¹H NMR (400 MHz, DMSO-d₆): δ 7.35-7.27 (m, 8H), 7.22-7.18 (m, 2H), 3.56 (s, 4H), 3.39 (t, J=6.4 Hz, 2H), 3.35-3.30 (m, 2H), 3.19 (s, 3H), 3.15-3.09 (m, 1H), 2.42-2.36 (m, 1H), 2.02-1.97 (m, 2H), 1.83-1.78 (m, 2H), 1.68-1.62 (m, 2H), 1.45-1.36 (m, 2H), 0.99-0.91 (m, 2H). LCMS: (Method C) 368.3 (M+H)

### Step 3: (1r,4r)-4-(3-methoxypropoxy)cyclohexan-1-amine

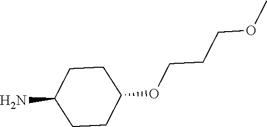

To a stirred solution of (1r,4r)-N,N-dibenzyl-4-(3-methoxypropoxy)cyclohexan-1-amine (750 mg, 2.04 mmol) in absolute ethanol (20 mL), Pd(OH)₂/C (150 mg, 20%) was added at RT and the mixture was stirred for overnight under hydrogen at RT. After completion (monitored by LCMS), the reaction mixture was filtered through Celite bed. The filtrate was evaporated under vacuum to afford the title compound which was used in the next step without further purification. Yield: 91% (350 mg, colorless liquid). ¹H NMR (400 MHz, DMSO-d₆): δ 3.44-3.39 (m, 2H), 3.33-3.33 (m, 3H), 3.21 (s, 3H), 3.16-3.08 (m, 1H), 1.90-1.85 (m, 2H), 1.69-1.63 (m, 4H), 1.17-0.96 (m, 4H). LCMS: (Method A) 188.2 (M+H)

### Step 4: N-((1r,4r)-4-(3-methoxypropoxy)cyclohexyl)-6-(thiazol-5-yl)-1H-indole-4-carboxamide

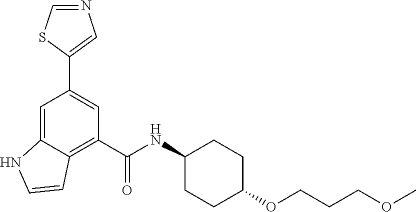 [SMILES: COCCCO[C@H]1CC[C@H](NC(=O)c2cc(-c3cncs3)cc3[nH]ccc23)CC1]

To a stirred solution of 6-(thiazol-5-yl)-1H-indole-4-carboxylic acid (80 mg, 0.32 mmol) in DMF (3 mL) were added HATU (249 mg, 0.65 mmol) and DIPEA (127 mg, 0.98 mmol) at RT. After stirring for 5 min, a solution of (1r,4r)-4-(3-methoxypropoxy)cyclohexan-1-amine (59.5 mg, 0.49 mmol) in DMF (0.5 mL) was added at RT and the reaction mixture was stirred at RT for 16 h. After completion (monitored by TLC), the reaction mixture was quenched with water (10 mL) and extracted with EtOAc (3×15 mL). The combined organic layer was washed with water (10 mL), brine (10 mL), dried over anhydrous Na₂SO₄ and concentrated under vacuum. The resulting crude residue was purified by flash chromatography on Biotage Isolera (230-400 mesh silica gel, eluent: 10-50% EtOAc in petroleum ether) to afford the title compound. Yield: 31% (41 mg, yellow solid). ¹H NMR (400 MHz, DMSO-d₆): δ 11.39 (s, 1H), 9.05 (s, 1H), 8.31 (s, 1H), 8.17 (d, J=7.6 Hz, 1H), 7.76 (s, 1H), 7.66 (d, J=1.2 Hz, 1H), 7.51-7.49 (m, 1H), 6.81 (s, 1H), 3.85-3.81 (m, 1H), 3.48-3.41 (m, 2H), 3.40-3.36 (m, 2H), 3.30-3.19 (m, 4H), 2.04-2.01 (m, 2H), 1.94-1.91 (m, 2H), 1.74-1.67 (m, 2H), 1.46-1.38 (m, 2H), 1.30-1.21 (m, 2H). LCMS: (Method C) 414.1 (M+H)

### Example 38: N-((1r,4r)-4-(2-methoxyethoxy)cyclohexyl)-6-(thiazol-5-yl)-1H-indole-4-carboxamide

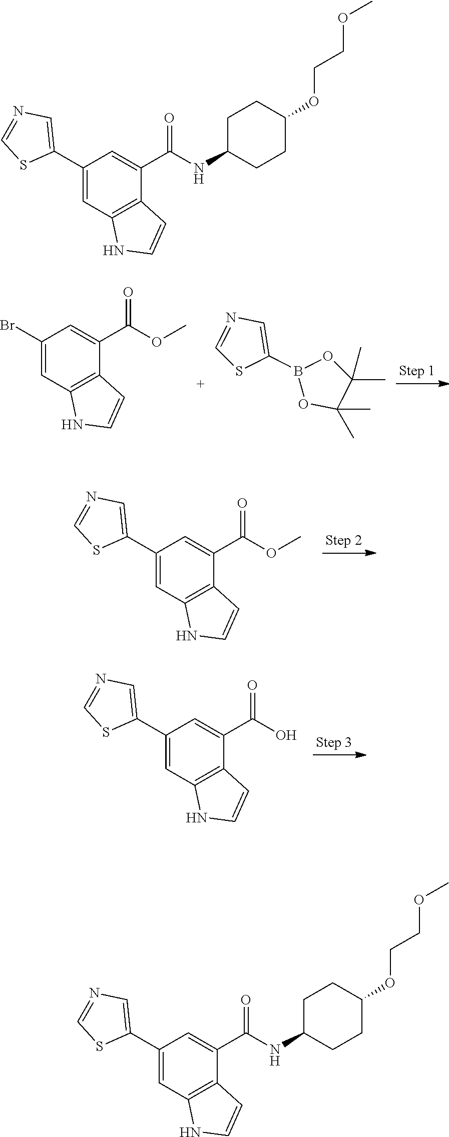

### Step 1: methyl 6-(thiazol-5-yl)-1H-indole-4-carboxylate

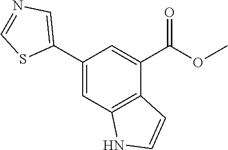 [SMILES: COC(=O)c1cc(-c2cncs2)cc2[nH]ccc12]

To a stirred solution of methyl 6-bromo-1H-indole-4-carboxylate (2.0 g, 7.87 mmol) in a mixture of 1,4-dioxane (50 mL) and water (5 mL) at RT, 5-(4,4,5,5-tetramethyl-1,3,2-dioxaborolan-2-yl)thiazole (2.15 g, 10.23 mmol), CuI (149 mg, 0.79 mmol) and potassium carbonate (3.25 g, 23.61 mmol) were added at RT and the mixture was purged with nitrogen gas for 2 min. Then Pd(dppf)Cl₂.DCM (275 mg, 0.39 mmol) was added and the reaction mixture was heated at 120° C. for 16 h. After completion, the reaction mixture was filtered through Celite and the Celite-bed was washed with DCM (100 mL). The combined filtrate was concentrated under vacuum and the resulting crude residue was purified by flash chromatography on Biotage Isolera (50% EtOAc in hexanes) to get the title compound. Yield: 59% (1.2 g, off white solid). ¹H NMR (400 MHz, DMSO-d₆): δ 11.63 (s, 1H), 9.08 (d, J=0.8 Hz, 1H), 8.33 (d, J=0.4 Hz, 1H), 7.98-7.97 (m, 2H), 7.64-7.63 (m, 1H), 6.97-6.96 (m, 1H), 3.94 (s, 3H). LCMS: (Method C) 259.0 (M+H)

### Step 2: 6-(thiazol-5-yl)-1H-indole-4-carboxylic acid

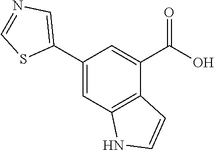 [SMILES: O=C(O)c1cc(-c2cncs2)cc2[nH]ccc12]

To a stirred solution of methyl 6-(thiazol-5-yl)-1H-indole-4-carboxylate (1.2 g, 4.65 mmol) in a mixture of THF (24 mL) and water (8 mL) at RT, was added NaOH (0.558 g, 13.95 mmol) and the reaction mixture was stirred at RT for 16 h. The reaction mixture was monitored by TLC. After complete consumption of the starting material, the reaction mixture was concentrated under vacuum. The residue was dissolved in 1,4-dioxane (20 mL), acidified with HCl in 1,4-dioxane (5 mL, 4 M) and then concentrated under reduced pressure to get the title compound Yield: 100% (1.4 g crude yield, 100% yield is 1.13 g, Pale yellow solid). ¹H NMR (400 MHz, DMSO-d₆): δ 11.82 (s, 1H), 9.13 (s, 1H), 8.33 (s, 1H), 7.96 (s, 2H), 7.59-7.58 (m, 1H), 6.95 (s, 1H). LCMS: (Method C) 245.1 (M+H)

### Step 3: N-((1r,4r)-4-(2-methoxyethoxy)cyclohexyl)-6-(thiazol-5-yl)-1H-indole-4-carboxamide

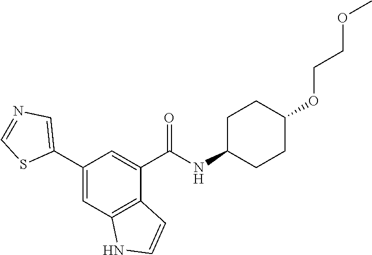 [SMILES: COCCO[C@H]1CC[C@H](NC(=O)c2cc(-c3cncs3)cc3[nH]ccc23)CC1]

To a stirred solution of 6-(thiazol-5-yl)-1H-indole-4-carboxylic acid (0.9 g, 3.68 mmol) in DMF (18 mL) at 0° C., were added DIPEA (1.98 mL, 11.0 mmol) and HATU (1.68 g, 4.42 mmol). After stirring for 5 min, a solution of (1r,4r)-4-(2-methoxyethoxy)cyclohexan-1-amine (0.765 g, 4.42 mmol) was added at 0° C. and the reaction mixture was stirred at RT for 16 h. The reaction mixture was monitored by TLC. After completion, the reaction mixture was diluted with water (50 mL) and the resulting suspension was extracted with EtOAc (2×100 mL). The combined organic layer was washed with water (50 mL), brine (50 mL), dried over anhydrous Na₂SO₄ and concentrated under vacuum. The resulting crude residue was purified by flash chromatography on Biotage Isolera (230-400 mesh silica gel, eluent: 3% MeOH in DCM) followed by reverse phase purification (Method A). The collected fraction was concentrated under reduced pressure. The residue was diluted with 10% MeOH in DCM (10 mL) and washed with 10% aq. NaHCO₃ solution (4 mL), brine solution and water. The organic layer was dried over anhydrous Na₂SO₄, filtered, concentrated under reduced pressure and lyophilized to get the title compound. Yield: 30% (0.447 g, off white solid). ¹H NMR (400 MHz, DMSO-d₆): δ 11.39 (s, 1H), 9.06 (s, 1H), 8.31 (s, 1H), 8.17 (d, J=7.6 Hz, 1H), 7.76 (s, 1H), 7.66 (s, 1H), 7.50-7.49 (m, 1H), 6.81 (s, 1H), 3.86-3.84 (m, 1H), 3.56-3.53 (m, 2H), 3.45-3.42 (m, 2H), 3.30-3.26 (m, 4H), 2.05-2.02 (m, 2H), 1.94-1.91 (m, 2H), 1.46-1.37 (m, 2H), 1.30-1.22 (m, 2H). LCMS: (Method A) 400.0 (M+H)

### Example 39: N-(1-(2-fluorophenyl)ethyl)-6-(thiazol-5-yl)-1H-indole-4-carboxamide

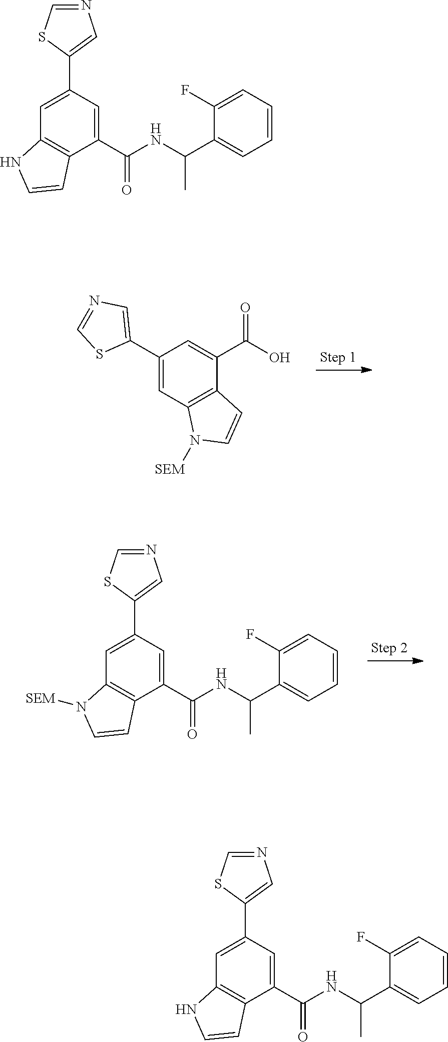 [SMILES: CC(NC(=O)c1cc(-c2cncs2)cc2[nH]ccc12)c1ccccc1F ; C[Si](C)(C)CCOCn1ccc2c(C(=O)O)cc(-c3cncs3)cc21 ; CC(NC(=O)c1cc(-c2cncs2)cc2c1ccn2COCC[Si](C)(C)C)c1ccccc1F ; CC(NC(=O)c1cc(-c2cncs2)cc2[nH]ccc12)c1ccccc1F]

### Step 1: N-(1-(2-fluorophenyl)ethyl)-6-(thiazol-5-yl)-1-((2-(trimethylsilyl)ethoxy)methyl)-1H-indole-4-carboxamide

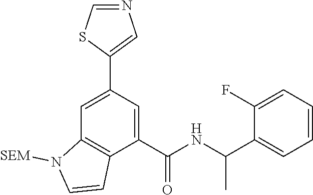 [SMILES: CC(NC(=O)c1cc(-c2cncs2)cc2c1ccn2COCC[Si](C)(C)C)c1ccccc1F]

To a stirred solution of 6-(thiazol-5-yl)-1-((2-(trimethylsilyl)ethoxy)methyl)-1H-indole-4-carboxylic acid (0.2 g, 0.53 mmol) in DMF (2 mL) at 0° C., HATU (304 mg, 0.80 mmol) was added followed by DIPEA (0.29 mL, 1.60 mmol) under nitrogen atmosphere. The reaction was continued at 0° C. for 10 min, then 1-(2-fluorophenyl)ethan-1-amine (89 mg, 0.64 mmol) was added, and the reaction mixture was stirred for another 16 h at RT. The reaction mixture was diluted with ice-cold water (10 mL) and extracted with EtOAc (2×10 mL). The combined organic layer was washed with water (2×10 mL), brine solution (2×10 mL), dried over anhydrous Na₂SO₄, filtered and the filtrate was concentrated under vacuum. The resulting crude residue was used for the next step without further purification. Yield: 83% (0.22 g, off white solid). LCMS: (Method A) 496.2 (M+H)

### Step 2: N-(1-(2-fluorophenyl)ethyl)-6-(thiazol-5-yl)-1H-indole-4-carboxamide

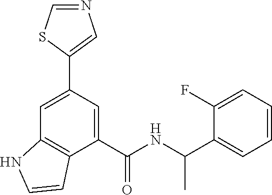 [SMILES: CC(NC(=O)c1cc(-c2cncs2)cc2[nH]ccc12)c1ccccc1F]

To a stirred solution of N-(1-(2-fluorophenyl)ethyl)-6-(thiazol-5-yl)-1-((2-(trimethylsilyl)ethoxy)methyl)-1H-indole-4-carboxamide (220 mg, 0.44 mmol) in THF (10 mL) at RT, a solution of TBAF in THF (1.33 mL, 1.33 mmol, 1.0 M) was added slowly. The reaction mixture was heated to reflux for 16 h. After completion, the reaction mixture was cooled to RT and concentrated under reduced pressure. The crude residue was suspended with ice-cold water (10 mL) and extracted with EtOAc (2×10 mL). The resulting organic layer was washed with water (2×10 mL), brine solution (2×10 mL), dried over anhydrous Na₂SO₄, filtered and the filtrate was concentrated under vacuum. The crude residue was purified by Prep-HPLC (Method A). The prep fraction was collected, concentrated under reduced pressure. The residue was diluted with 10% MeOH in DCM (10 mL), washed with 10% aq. NaHCO₃ solution (4 mL), brine solution and water. The organic layer was dried over anhydrous Na₂SO₄, filtered, concentrated under reduced pressure and lyophilized to get the title compound. Yield: 11% (17.93 mg, white solid). ¹H NMR (400 MHz, DMSO-d₆): δ 11.43 (s, 1H), 9.07 (s, 1H), 8.87 (d, J=7.6 Hz, 1H), 8.35 (s, 1H), 7.81 (s, 1H), 7.79 (s, 1H), 7.55 (t, J=6.7 Hz, 1H), 7.50 (s, 1H), 7.33-7.28 (m, 1H), 7.22-7.16 (m, 2H), 6.79 (s, 1H), 5.45-5.40 (m, 1H), 1.52 (d, J=7.0 Hz, 3H). LCMS: (Method C) 366.2 (M+H)

### Example 40: N-((1R,2S)-2-methylcyclohexyl)-6-(thiazol-5-yl)-1H-indole-4-carboxamide

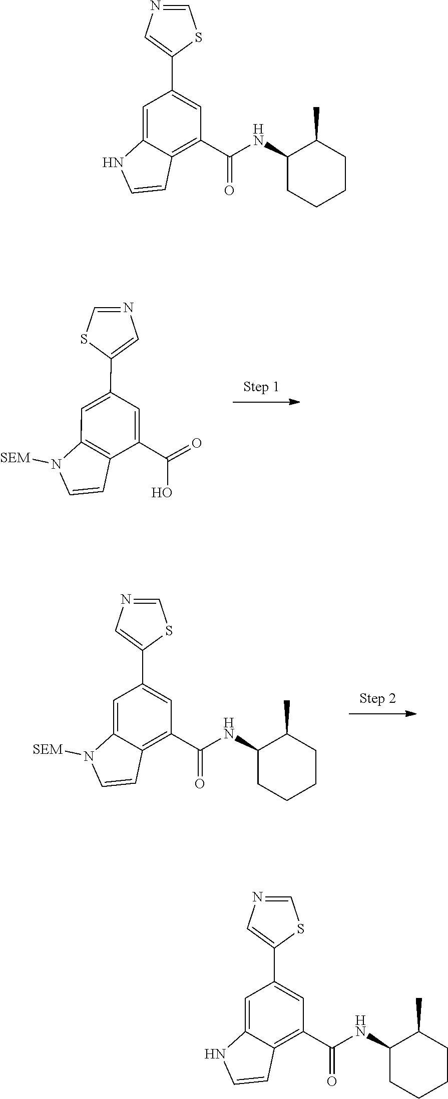 [SMILES: C[C@H]1CCCC[C@H]1NC(=O)c1cc(-c2cncs2)cc2[nH]ccc12 ; C[Si](C)(C)CCOCn1ccc2c(C(=O)O)cc(-c3cncs3)cc21 ; C[C@H]1CCCC[C@H]1NC(=O)c1cc(-c2cncs2)cc2c1ccn2COCC[Si](C)(C)C ; C[C@H]1CCCC[C@H]1NC(=O)c1cc(-c2cncs2)cc2[nH]ccc12]

### Step 1: N-((1R,2S)-2-methylcyclohexyl)-6-(thiazol-5-yl)-1-((2-(trimethylsilyl) ethoxy) methyl)-1H-indole-4-carboxamide

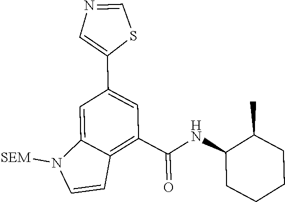 [SMILES: C[C@H]1CCCC[C@H]1NC(=O)c1cc(-c2cncs2)cc2c1ccn2COCC[Si](C)(C)C]

To a stirred solution of 6-(thiazol-5-yl)-1-((2-(trimethylsilyl)ethoxy)methyl)-1H-indole-4-carboxylic acid (200 mg, 0.53 mmol) in DMF (2 mL) HATU (304 mg, 0.80 mmol) and DIPEA (0.24 mL, 1.33 mmol) were added under nitrogen atmosphere at 0° C. and then after 2 min, (1R,2S)-2-methylcyclohexan-1-amine (72 mg, 0.64 mmol) was added. Further the reaction mixture was stirred for 16 h at RT. After completion of reaction, the reaction mixture was diluted with ice cold water (10 mL) and extracted with EtOAc (50 mL). The organic solution was washed with brine solution (50 mL), water (25 mL), dried over anhydrous Na₂SO₄ and concentrated under vacuum. The resulting crude product was used for next step without further purification. Yield: 84% (210 mg, Brown gummy solid). LCMS: (Method C) 470.2 (M+H)

### Step 2: N-((1R,2S)-2-methylcyclohexyl)-6-(thiazol-5-yl)-1H-indole-4-carboxamide

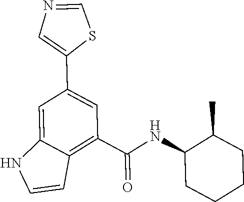 [SMILES: C[C@H]1CCCC[C@H]1NC(=O)c1cc(-c2cncs2)cc2[nH]ccc12]

To a stirred solution of N-((1R,2S)-2-methylcyclohexyl)-6-(thiazol-5-yl)-1-((2-(trimethylsilyl)ethoxy)methyl)-1H-indole-4-carboxamide (200 mg, 0.42 mmol) in THF (5 mL) TBAF (1.0 M in THF) (1.27 mL, 1.27 mmol) was added at RT and stirred for 16 h at 70° C. After completion of reaction, the reaction mixture was quenched with ice cold water (10 mL) and extracted with EtOAc (50 mL). The organic solution was washed with brine solution (2×25 mL), dried over anhydrous Na₂SO₄ and concentrated under vacuum. The resulting crude was purified by Prep. HPLC (Method B). The prep-fraction was collected, concentrated under reduced pressure. The resulting solid was dissolved in 10% MeOH in DCM (10 mL) washed with water, dried over anhydrous Na₂SO₄, concentrated under reduced pressure and lyophilized to get the title compound. Yield: 13% (19.29 mg, off white solid). ¹HNMR (400 MHz, DMSO-d₆): 11.41 (s, 1H), 9.05 (s, 1H), 8.31 (s, 1H), 7.94 (d, J=8.4 Hz, 1H), 7.77 (s, 1H), 7.6 (d, J=1.4 Hz, 1H), 7.50 (t, J=2.6 Hz, 1H), 6.70 (s, 1H), 4.17-4.14 (m, 1H), 3.51-3.45 (m, 1H), 2.08-2.00 (m, 1H), 1.76-1.72 (m, 2H), 1.67-1.64 (m, 3H), 1.48-1.43 (m, 2H), 0.9 (d, J=7.0 Hz, 3H). LCMS: (Method C) 340.3 (M+H)

### Example 41: N-((1r,4r)-4-hydroxycyclohexyl)-6-(thiazol-5-yl)-1H-indole-4-carboxamide

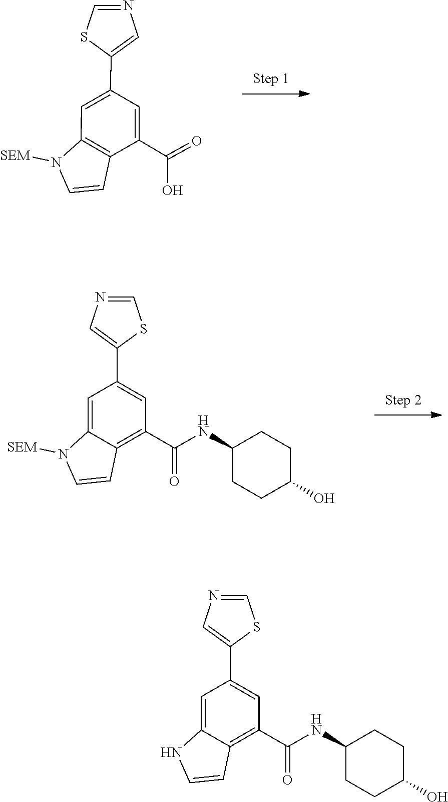 [SMILES: C[Si](C)(C)CCOCn1ccc2c(C(=O)O)cc(-c3cncs3)cc21 ; C[Si](C)(C)CCOCn1ccc2c(C(=O)N[C@H]3CC[C@H](O)CC3)cc(-c3cncs3)cc21 ; O=C(N[C@H]1CC[C@H](O)CC1)c1cc(-c2cncs2)cc2[nH]ccc12]

### Step 1: N-((1r,4r)-4-hydroxycyclohexyl)-6-(thiazol-5-yl)-1-((2-(trimethylsilyl)ethoxy)methyl)-1H-indole-4-carboxamide

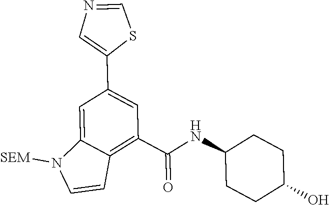 [SMILES: C[Si](C)(C)CCOCn1ccc2c(C(=O)N[C@H]3CC[C@H](O)CC3)cc(-c3cncs3)cc21]

To a stirred solution of 6-(thiazol-5-yl)-1-((2-(trimethylsilyl)ethoxy)methyl)-1H-indole-4-carboxylic acid (0.3 g, 0.80 mmol) in DMF (5 mL) at 0° C., HATU (0.45 g, 1.20 mmol) followed by DIPEA (0.36 mL, 2.09 mmol) were added under nitrogen atmosphere and the reaction mixture was stirred at 0° C. for 5 min. Then trans-4-aminocyclohexan-1-ol (0.13 g, 1.20 mmol) was added and the reaction mixture was stirred for 16 h at RT. After completion the reaction mixture was diluted with ice cold water (10 mL) and then extracted with EtOAc (50 mL). The combined organic layer was washed with water (25 mL), brine solution (50 mL), dried over anhydrous Na₂SO₄ and filtered. The filtrate was concentrated under vacuum to obtain the title compound which was used in the next step without purification. Yield: 79% (0.30 g, brown gummy solid). LCMS: (Method A) 472.1 (M+H)

### Step 2: N-((1r,4r)-4-hydroxycyclohexyl)-6-(thiazol-5-yl)-1H-indole-4-carboxamide

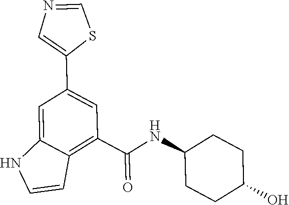 [SMILES: O=C(N[C@H]1CC[C@H](O)CC1)c1cc(-c2cncs2)cc2[nH]ccc12]

To a stirred solution of N-((1r,4r)-4-hydroxycyclohexyl)-6-(thiazol-5-yl)-1-((2-(trimethylsilyl)ethoxy)methyl)-1H-indole-4-carboxamide (0.3 g, 0.63 mmol) in THF (5 mL) at RT, a solution of TBAF in THF (1.90 mL, 1.0 M, 1.90 mmol) was added and the reaction mixture was stirred for 16 h at 70° C. After completion (starting material was consumed according to TLC), the reaction mixture was diluted with ice cold water (10 mL) and extracted with EtOAc (50 mL). The resulting organic layer was washed with brine solution (50 mL), dried over anhydrous Na₂SO₄, filtered and the filtrate was concentrated under vacuum. The resulting crude residue was purified by Prep-HPLC (Method A). The prep-fraction was collected, concentrated under reduced pressure, diluted with 10% MeOH in DCM (10 mL) and washed with water. The organic layer was dried over anhydrous Na₂SO₄, filtered, concentrated under reduced pressure and lyophilized to get the title compound. Yield: 14% (31.35 mg, off white solid). ¹HNMR (400 MHz, DMSO-d₆): 11.39 (s, 1H), 9.06 (s, 1H), 8.31 (s, 1H), 8.14 (d, J=7.9 Hz, 1H), 7.76 (s, 1H), 7.66 (d, J=1.3 Hz, 1H), 7.51-7.50 (m, 1H), 6.82 (s, 1H), 4.57 (d, J=4.4 Hz, 1H), 3.83-3.77 (m, 1H), 3.44-3.36 (m, 1H), 1.90-1.87 (m, 4H), 1.46-1.37 (m, 2H), 1.32-1.23 (m, 2H). LCMS: (Method C) 342.2 (M+H)

### Example 42: N-(1-methoxy-2-methylpropan-2-yl)-6-(thiazol-5-yl)-1H-indole-4-carboxamide

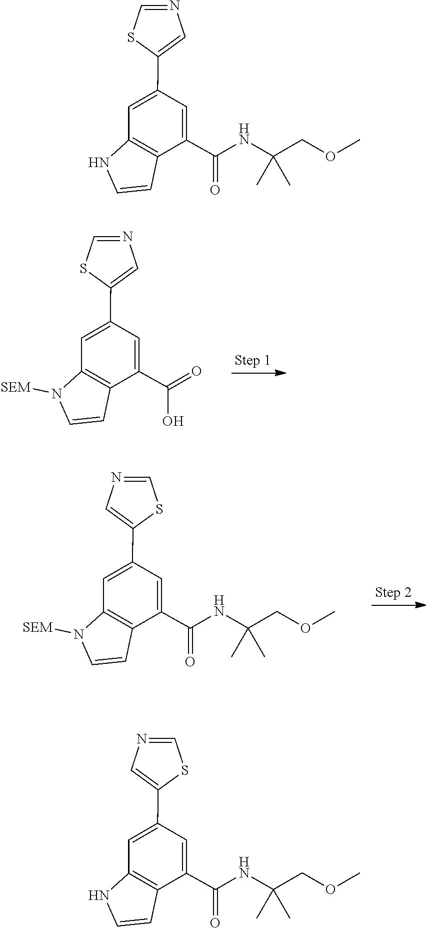

### Step-1: N-(1-methoxy-2-methylpropan-2-yl)-6-(thiazol-5-yl)-1-((2-(trimethylsilyl)ethoxy)methyl)-1H-indole-4-carboxamide

To a stirred solution of 6-(thiazol-5-yl)-1-((2-(trimethylsilyl)ethoxy)methyl)-1H-indole-4-carboxylic acid (200 mg, 0.534 mmol) in DMF (2 mL) were added DIPEA (0.28 mL, 1.60 mmol) and HATU (304 mg, 0.802 mmol) at 0° C. After stirring for 5 min a solution of 1-methoxy-2-methylpropan-2-amine (0.066 g, 0.641 mmol) in DMF (0.5 mL) was added at same temperature and further reaction was stirred at RT for 16 h. The reaction mixture was monitored by TLC and quenched with water (5 mL). The resulting suspension was extracted with EtOAc (2×10 mL). The combined organic layer was washed with water (10 mL), brine (10 mL), dried over anhydrous Na₂SO₄ and solvent was evaporated under vacuum. The resulting crude product was used for next step without further purification. Yield: 81.62% (0.2 g, Brown solid) LCMS: (Method C) 460.2 (M+H)

### Step-2: N-(1-methoxy-2-methylpropan-2-yl)-6-(thiazol-5-yl)-1H-indole-4-carboxamide

To a stirred solution of N-(1-methoxy-2-methylpropan-2-yl)-6-(thiazol-5-yl)-1-((2-(trimethylsilyl)ethoxy)methyl)-1H-indole-4-carboxamide (190 mg, 0.413 mmol) in THF (5 mL) was added at 1M TBAF in THF (1.24 mL,1.24 mmol) and RT and heated at 70° C. for 16 h. After completion of the reaction, the reaction mixture was evaporated under vacuum and the resulting crude product which was purified by Prep-HPLC. The collected fraction was concentered under vacuum. The resulting residue was dissolved in DCM and neutralized with 10% NaHCO₃ aqueous solution. The organic phase was washed with water, brine, dried over anhydrous Na₂SO₄ and evaporated to afford the title compound. Yield: 10% (13.38 mg, off white solid). ¹HNMR (400 MHz, DMSO-d₆): δ 11.42 (s, 1H), 9.04 (s, 1H), 8.30 (s, 1H), 7.76 (s, 1H), 7.60 (d, J=1.4 Hz, 1H), 7.56 (s, 1H), 7.51 (d, J=2.3 Hz, 1H), 6.75 (d, J=2.7 Hz, 1H), 3.57 (s, 2H), 3.32 (s, 3H), 1.40 (s, 6H). LCMS: (Method C) 330.2 (M+H)

### Example 43: N-(2-hydroxy-2-methylpropyl)-6-(thiazol-5-yl)-1H-indole-4-carboxamide

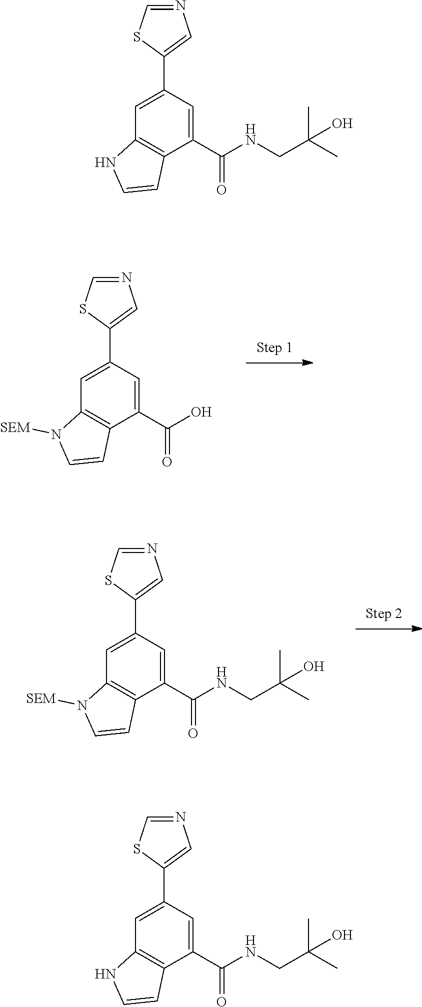 [SMILES: CC(C)(O)CNC(=O)c1cc(-c2cncs2)cc2[nH]ccc12 ; C[Si](C)(C)CCOCn1ccc2c(C(=O)O)cc(-c3cncs3)cc21 ; CC(C)(O)CNC(=O)c1cc(-c2cncs2)cc2c1ccn2COCC[Si](C)(C)C ; CC(C)(O)CNC(=O)c1cc(-c2cncs2)cc2[nH]ccc12]

### Step-1: N-(2-hydroxy-2-methylpropyl)-6-(thiazol-5-yl)-1-((2-(trimethylsilyl)ethoxy)methyl)-1H-indole-4-carboxamide

To a stirred solution of 6-(thiazol-5-yl)-1-((2-(trimethylsilyl)ethoxy)methyl)-1H-indole-4-carboxylic acid (200 mg, 0.534 mmol) in DMF (2 mL) were added DIPEA (0.28 mL, 1.60 mmol) and HATU (304 mg, 0.802 mmol) at 0° C. After stirring for 5 min a solution of 1-amino-2-methylpropan-2-ol (57 mg, 0.640 mmol) in DMF (0.5 mL) was added at same temperature and stirred at RT for 16 h. The reaction mixture was monitored by TLC and quenched with water (5 mL). The resulting suspension was extracted with EtOAc (2×10 mL). The combined organic layer was washed with water (10 mL), brine (10 mL), dried over anhydrous Na₂SO₄ and solvent was evaporated under vacuum. The resulting crude product was used for next step without further purification. Yield: 86.9% (0.2 g, Brown solid). LCMS: (Method C) 446.20 (M+H),

### Step-2: N-(2-hydroxy-2-methylpropyl)-6-(thiazol-5-yl)-1H-indole-4-carboxamide

To a stirred solution of N-(2-hydroxy-2-methylpropyl)-6-(thiazol-5-yl)-1-((2-(trimethylsilyl)ethoxy)methyl)-1H-indole-4-carboxamide (190 mg, 0.426 mmol) in THF (5 mL) was added at 1M TBAF in THF (1.28 mL 1.28 mmol) and heated at 70° C. for 16 h. After completion of the reaction, the reaction mixture was evaporated under vacuum and the resulting crude product which was purified by Prep-HPLC (Method A). The collected fraction was concentered under vacuum. The resulting residue was dissolved in DCM and neutralized with 10% NaHCO₃ aqueous solution. The organic phase was washed with water, brine, dried over anhydrous Na₂SO₄ and evaporated to afford the title compound. Yield: 6% (7.78 mg, off white solid). ¹HNMR 400 MHz, DMSO-d₆): δ 11.44 (s, 1H), 9.05 (s, 1H), 8.32 (s, 1H), 8.13 (t, J=5.6 Hz, 1H), 7.79 (s, 1H), 7.74 (d, J=1.6 Hz, 1H), 7.53 (t, J=2.6 Hz, 1H), 6.82 (t, J=2.0 Hz, 1H), 4.62 (s, 1H), 3.32 (s, 2H), 1.16 (s, 6H). LCMS: (Method C) 316.0 (M+H)

### Example 44: N-(adamantan-1-yl)-6-(thiazol-5-yl)-1H-indole-4-carboxamide

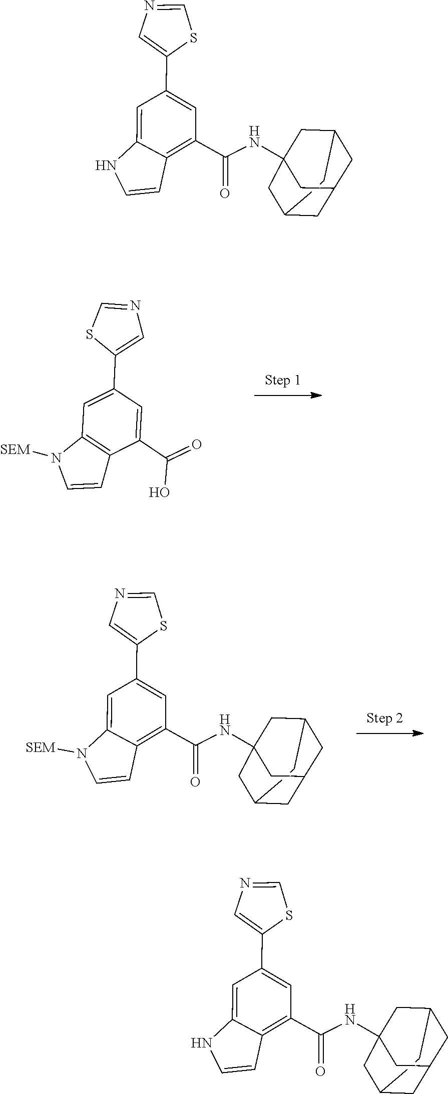 [SMILES: CCC1CC2CC(C)CC(NC(=O)c3cc(-c4cncs4)cc4[nH]ccc34)(C1)C2 ; C[Si](C)(C)CCOCn1ccc2c(C(=O)O)cc(-c3cncs3)cc21 ; CCC1CC2CC(C)CC(NC(=O)c3cc(-c4cncs4)cc4c3ccn4COCC[Si](C)(C)C)(C1)C2 ; CCC1CC2CC(C)CC(NC(=O)c3cc(-c4cncs4)cc4[nH]ccc34)(C1)C2]

### Step 1: N-(adamantan-1-yl)-6-(thiazol-5-yl)-1-((2-(trimethylsilyl)ethoxy)methyl)-1H-indole-4-carboxamide

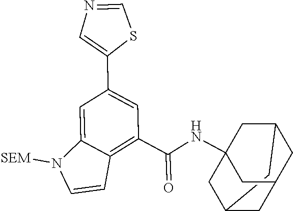 [SMILES: C[Si](C)(C)CCOCn1ccc2c(C(=O)NC34CC5CC(CC(C5)C3)C4)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (5 mL) at 0° C., HATU (0.30 g, 0.79 mmol) followed by DIPEA (0.24 mL, 1.33 mmol) were added under nitrogen atmosphere and then reaction mixture was stirred at 0° C. for 5 min. Then adamantan-1-amine (0.09 g, 0.64 mmol) was added and the reaction mixture was stirred for 16 h at RT. After completion (starting material was consumed according to TLC), the reaction mixture was diluted with ice cold water (5 mL) and extracted with EtOAc (50 mL). The resulting organic layer was washed with water (25 mL), brine solution (50 mL), dried over anhydrous Na₂SO₄, filtered and the filtrate was concentrated under vacuum to get the title compound which was used in the next step without purification. Yield: 73.6% (0.2 g, brown gummy solid). LCMS: (Method A) 508.00 (M+H)

### Step 2: N-(adamantan-1-yl)-6-(thiazol-5-yl)-1H-indole-4-carboxamide

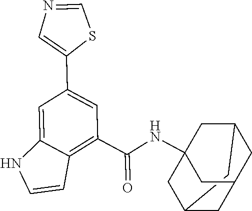 [SMILES: O=C(NC12CC3CC(CC(C3)C1)C2)c1cc(-c2cncs2)cc2[nH]ccc12]

To a stirred solution of N-(adamantan-1-yl)-6-(thiazol-5-yl)-1-((2-(trimethylsilyl)ethoxy)methyl)-1H-indole-4-carboxamide (0.2 g, 0.39 mmol) in THF (5 mL) at RT, a solution of TBAF in THF (1.18 mL, 1.0 M, 1.18 mmol) was added and the reaction mixture was stirred for 16 h at 70° C. After completion (starting material was consumed according to TLC), the reaction mixture was diluted with water (50 mL) and extracted with EtOAc (100 mL). The resulting organic layer was washed with brine solution (50 mL), dried over anhydrous Na₂SO₄, filtered and the filtrate was concentrated under vacuum. The resulting crude residue was purified by Prep-HPLC (Method A). The prep-fraction was collected, concentrated under reduced pressure, diluted with 10% MeOH in DCM (10 mL) and washed with aq. NaHCO₃ solution and water. The organic layer was dried over anhydrous Na₂SO₄, filtered, concentrated under reduced pressure and lyophilized to get the title compound. Yield: 29% (42.98 mg, off white solid). ¹H NMR (400 MHz, DMSO-d₆): 11.38 (s, 1H), 9.05 (d, J=0.8 Hz, 1H), 8.31 (s, 1H), 7.74 (s, 1H), 7.58-7.57 (m, 2H), 7.50 (d, J=3.2 Hz, 1H), 6.74 (d, J=2.8 Hz, 1H), 2.15-2.12 (m, 6H), 2.10-2.05 (m, 3H), 1.69 (s, 6H). LCMS: (Method C) 378.2 (M+H)

### Example 45: N-(tetrahydro-2H-pyran-4-yl)-6-(thiazol-5-yl)-1H-indole-4-carboxamide

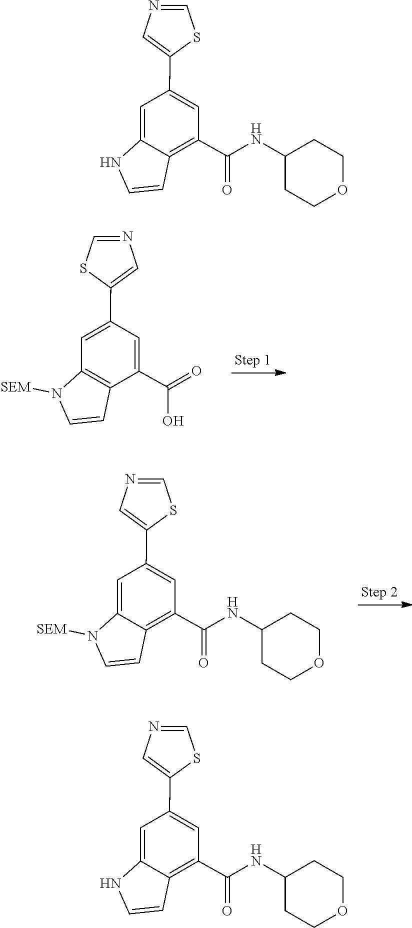

### Step 1: N-(tetrahydro-2H-pyran-4-yl)-6-(thiazol-5-yl)-1-((2-(trimethylsilyl)ethoxy)methyl)-1H-indole-4-carboxamide

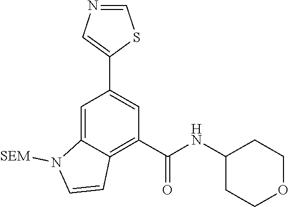 [SMILES: C[Si](C)(C)CCOCn1ccc2c(C(=O)NC3CCOCC3)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (5 mL) at 0° C., HATU (0.30 g, 0.80 mmol) followed by DIPEA (0.24 mL, 1.33 mmol) were added under nitrogen atmosphere and the reaction mixture was stirred at 0° C. for 5 min. Then tetrahydro-2H-pyran-4-amine (0.06 g, 0.64 mmol) was added and the reaction mixture was stirred for 16 h at RT. After completion (starting material was consumed according to TLC), the reaction mixture was diluted with ice cold water (20 mL) and extracted with EtOAc (50 mL). The resulting organic solution was washed with water (25 mL), brine solution (30 mL), dried over anhydrous Na₂SO₄, filtered and the filtrate was concentrated under vacuum to get the title compound which was used in next step without purification. Yield: 82% (0.2 g, brown gummy solid). LCMS: (Method A) 458.00 (M+H)

### Step 2: N-(tetrahydro-2H-pyran-4-yl)-6-(thiazol-5-yl)-1H-indole-4-carboxamide

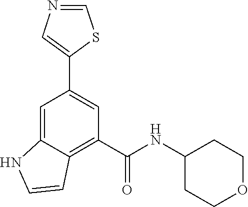 [SMILES: O=C(NC1CCOCC1)c1cc(-c2cncs2)cc2[nH]ccc12]

To a stirred solution of N-(tetrahydro-2H-pyran-4-yl)-6-(thiazol-5-yl)-1-((2-(trimethylsilyl)ethoxy)methyl)-1H-indole-4-carboxamide (0.2 g, 0.43 mmol) in THF (5 mL) at RT, a solution of TBAF in THF (1.31 mL, 1.0 M, 1.31 mmol) was added and the redaction mixture was stirred for 16 h at 70° C. After completion (starting material was consumed according to TLC), the reaction mixture was diluted with aq. 10% NaHCO₃ solution (20 mL) and extracted with EtOAc (50 mL). The resulting organic layer was washed with water (50 mL), brine solution (20 mL), dried over anhydrous Na₂SO₄, filtered and the filtrate was concentrated under vacuum. The resulting crude residue was purified by Prep-HPLC. (Method A). The prep-fraction was collected, concentrated under reduced pressure, diluted with 10% MeOH in DCM (10 mL) and washed with aq. NaHCO₃ solution and water. The organic layer was dried over anhydrous Na₂SO₄, filtered, concentrated under reduced pressure and lyophilized to get the title compound. Yield: 13% (18.28 mg, off white solid). ¹H NMR (400 MHz, DMSO-d₆): 11.41 (s, 1H), 9.06 (d, J=0.4 Hz, 1H), 8.32 (d, J=0.4 Hz, 1H), 8.29 (d, J=7.6 Hz, 1H), 7.78 (s, 1H), 7.70 (d, J=1.6 Hz, 1H), 7.52-7.50 (m, 1H), 6.83 (s, 1H), 4.11-4.04 (m, 1H), 3.93-3.88 (m, 2H), 3.45-3.39 (m, 2H), 1.85-1.81 (m, 2H), 1.68-1.58 (m, 2H). LCMS: (Method C) 328.2 (M+H)

### Example 46: N-((1r,3r)-3-hydroxycyclobutyl)-6-(thiazol-5-yl)-1H-indole-4-carboxamide

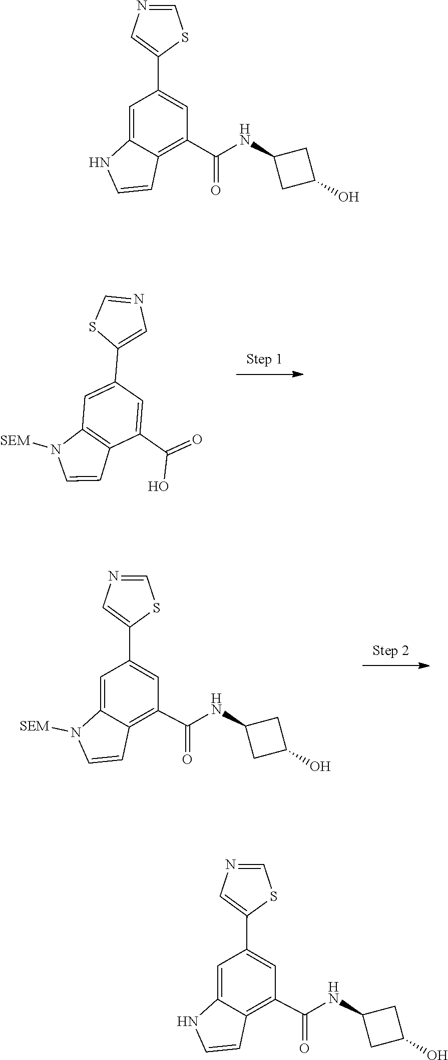 [SMILES: O=C(N[C@H]1C[C@H](O)C1)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)N[C@H]3C[C@H](O)C3)cc(-c3cncs3)cc21 ; O=C(N[C@H]1C[C@H](O)C1)c1cc(-c2cncs2)cc2[nH]ccc12]

### Step 1: N-((1r,3r)-3-hydroxycyclobutyl)-6-(thiazol-5-yl)-1-((2-(trimethylsilyl)ethoxy)methyl)-1H-indole-4-carboxamide

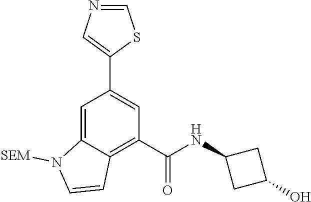 [SMILES: C[Si](C)(C)CCOCn1ccc2c(C(=O)N[C@H]3C[C@H](O)C3)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (5 mL) at 0° C., HATU (0.30 g, 0.80 mmol) followed by DIPEA (0.24 mL, 1.33 mmol) were added under nitrogen atmosphere and the reaction mixture was stirred at 0° C. for 5 min. Then (1r,3r)-3-aminocyclobutan-1-ol (0.07 g, 0.64 mmol) was added and then reaction was stirred for 16 h at RT. After completion (starting material was consumed according to TLC), the reaction mixture was diluted with ice-cold water (5 mL) and extracted with EtOAc (50 mL). The resulting organic layer was washed with water (2×25 mL), brine solution (50 mL), dried over anhydrous Na₂SO₄, filtered and the filtrate was concentrated under vacuum. The resulting crude residue was used for next step without purification. Yield: 84% (0.2 g, brown gummy solid). LCMS: (Method A) 444.00 (M+H)

### Step 2: N-((1r,3r)-3-hydroxycyclobutyl)-6-(thiazol-5-yl)-1H-indole-4-carboxamide

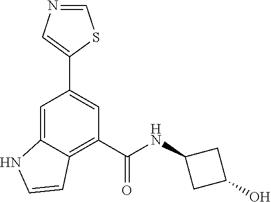 [SMILES: O=C(N[C@H]1C[C@H](O)C1)c1cc(-c2cncs2)cc2[nH]ccc12]

To a stirred solution of N-((1r,3r)-3-hydroxycyclobutyl)-6-(thiazol-5-yl)-1-((2-(trimethylsilyl)ethoxy)methyl)-1H-indole-4-carboxamide (0.2 g, 0.45 mmol) in THF (5 mL) at RT, a solution of TBAF in THF (1.35 mL, 1.0 M, 1.35 mmol) was added and the reaction mixture was stirred for 16 h at 70° C. After completion (starting material was consumed according to TLC), the reaction mixture was diluted with 10% NaHCO₃ solution (20 mL) and extracted with EtOAc (50 mL). The resulting organic layer was washed with water (20 mL), brine solution (20 mL), dried over anhydrous Na₂SO₄, filtered and the filtrate was concentrated under vacuum. The resulting crude residue was purified by Prep-HPLC (Method A). The prep-fraction was concentrated under reduced pressure, the resulting residue was diluted with 10% MeOH in DCM (10 mL) and washed with aq. NaHCO₃ solution and water. The organic layer was dried over anhydrous Na₂SO₄, filtered, concentrated under reduced pressure and lyophilized to get the title compound. Yield: 2% (3.65 mg, off white solid). ¹H NMR (400 MHz, DMSO-d₆): 11.41 (s, 1H), 9.06 (d, J=0.8 Hz, 1H), 8.57 (d, J=6.8 Hz, 1H), 8.32 (d, J=0.4 Hz, 1H), 7.77 (s, 1H), 7.70 (d, J=1.6 Hz, 1H), 7.51-7.49 (m, 1H), 6.83-6.81 (m, 1H), 5.04 (d, J=5.2 Hz, 1H), 4.53-4.48 (m, 1H), 4.38-4.33 (m, 1H), 2.34-2.30 (m, 2H), 2.27-2.19 (m, 2H), LCMS: (Method C) 314.1 (M+H)

### Example 47: N-cyclohexyl-6-(thiazol-5-yl)-1H-indole-4-carboxamide

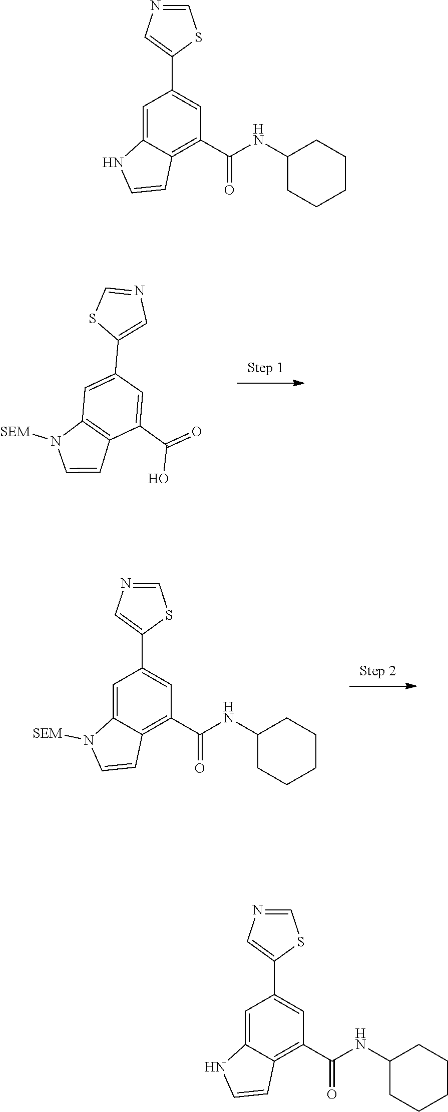 [SMILES: O=C(NC1CCCCC1)c1cc(-c2cncs2)cc2[nH]ccc12 ; C[Si](C)(C)CCOCn1ccc2c(C(=O)O)cc(-c3cncs3)cc21 ; C[Si](C)(C)CCOCn1ccc2c(C(=O)NC3CCCCC3)cc(-c3cncs3)cc21 ; O=C(NC1CCCCC1)c1cc(-c2cncs2)cc2[nH]ccc12]

### Step 1: N-cyclohexyl-6-(thiazol-5-yl)-1-((2-(trimethylsilyl)ethoxy)methyl)-1H-indole-4-carboxamide

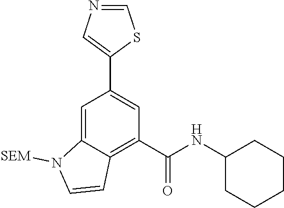 [SMILES: C[Si](C)(C)CCOCn1ccc2c(C(=O)NC3CCCCC3)cc(-c3cncs3)cc21]

To a stirred solution of 6-(thiazol-5-yl)-1-((2-(trimethylsilyl)ethoxy)methyl)-1H-indole-4-carboxylic acid (0.2 g, 0.53 mmol) in DMF (5 mL) at 0° C., were added HATU (0.30 g, 0.80 mmol) and DIPEA (0.24 mL, 1.33 mmol) under nitrogen atmosphere. After stirring for 10 min at 0° C., cyclohexanamine (0.06 g, 0.64 mmol) was added and the reaction mixture was stirred for 16 h at RT. After completion (starting material was consumed according to TLC), the reaction mixture was diluted with ice cold water (10 mL) and extracted with EtOAc (50 mL). The resulting organic layer was washed with water (2×25 mL), brine solution (20 mL), dried over anhydrous Na₂SO₄, filtered and the filtrate was concentrated under vacuum. The resulting crude residue was used for the next step without further purification. Yield: 82% (0.2 g, brown gummy solid). LCMS: (Method A) 456.0 (M+H)

### Step 2: N-cyclohexyl-6-(thiazol-5-yl)-1H-indole-4-carboxamide

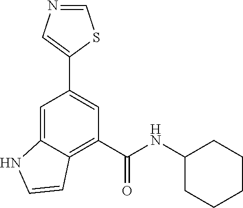 [SMILES: O=C(NC1CCCCC1)c1cc(-c2cncs2)cc2[nH]ccc12]

To a stirred solution of N-cyclohexyl-6-(thiazol-5-yl)-1-((2-(trimethylsilyl)ethoxy)methyl)-1H-indole-4-carboxamide (0.2 g, 0.43 mmol) in THF (5 mL) at RT, a solution of TBAF in THF (1.31 mL, 1.0 M, 1.31 mmol) was added and the reaction mixture was stirred for 16 h at 70° C. After completion (starting material was consumed according to TLC), the reaction mixture was diluted with 10% aq. NaHCO₃ solution (20 mL) and extracted with EtOAc (50 mL). The resulting organic layer was washed with water (2×50 mL), brine solution (20 mL), dried over anhydrous Na₂SO₄, filtered and the filtrate was concentrated under vacuum. The resulting crude residue was purified by Prep-HPLC (Method A). The prep-fraction was collected, concentrated under reduced pressure, diluted with 10% MeOH in DCM (10 mL) and washed with aq. NaHCO₃ solution and water. The organic layer was dried over anhydrous Na₂SO₄, filtered, concentrated under reduced pressure and lyophilized to get the title compound. Yield: 7% (10.88 mg, off white solid). ¹HNMR (400 MHz, DMSO-d₆): 11.39 (s, 1H), 9.06 (d, J=0.4 Hz, 1H), 8.31 (s, 1H), 8.16 (d, J=8.0 Hz, 1H), 7.76 (s, 1H), 7.67 (d, J=1.6 Hz, 1H), 7.50 (d, J=2.8 Hz, 1H), 6.82 (d, J=2.8 Hz, 1H), 3.84-3.80 (m, 1H), 1.91-1.88 (m, 2H), 1.77-1.72 (m, 2H), 1.65-1.62 (m, 1H), 1.42-1.26 (m, 4H), 1.20-1.12 (m, 1H), LCMS: (Method C) 326.2 (M+H)

### Example 48: N-((1s,4s)-4-(2-methoxyethoxy)cyclohexyl)-6-(thiazol-5-yl)-1H-indole-4-carboxamide

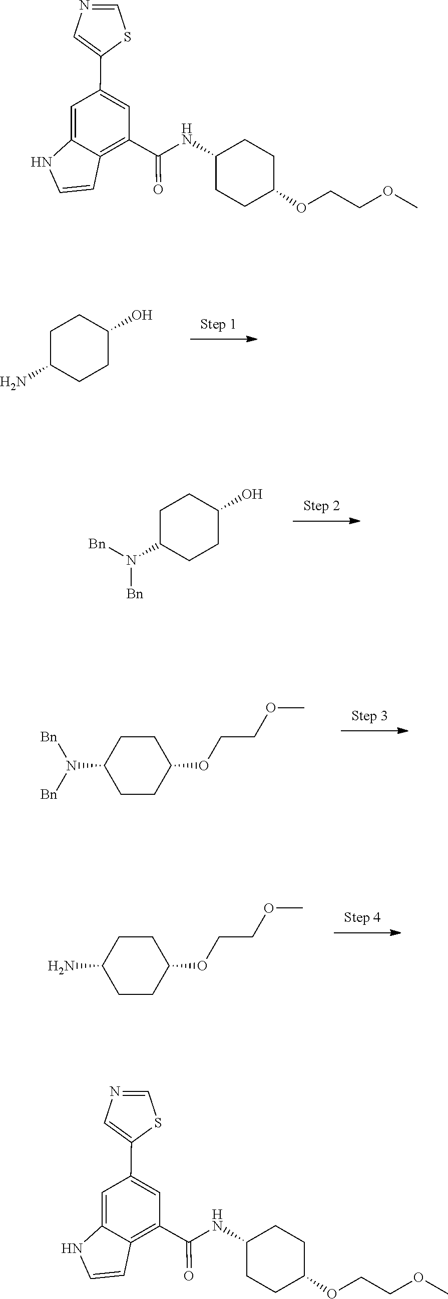

### Step 1: (1s,4s)-4-(dibenzylamino)cyclohexan-1-ol

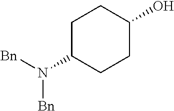

To a stirred solution of (1s,4s)-4-aminocyclohexan-1-ol hydrochloride (1.0 g, 6.59 mmol) in ACN (15 mL) at RT, K₂CO₃ (2.73 g, 19.78 mmol) and benzyl bromide (1.56 mL, 13.19 mmol) were added and the reaction mixture was heated at 70° C. for 5 h. After completion (starting material was consumed according to TLC analysis), the reaction mixture was diluted with ice cold water (100 mL) and extracted with MTBE (200 mL). The resulting organic layer was washed with water (2×100 mL), brine solution (200 mL), dried over anhydrous Na₂SO₄, filtered and the filtrate was concentrated under vacuum. The resulting crude residue was used for next the step without further purification. Yield: 76% (1.5 g, pale yellow oil). ¹H NMR (400 MHz, DMSO-d₆): 7.36-7.34 (m, 10H), 4.26 (d, J=3.6 Hz 1H), 3.75-3.70 (m, 1H), 3.59 (s, 4H), 2.41-2.35 (m, 1H), 1.79-1.66 (m, 4H), 1.55-1.51 (m, 2H), 1.25-1.22 (m, 2H). LCMS: (Method C) 296.2 (M+H)

### Step 2: (1s,4s)-N,N-dibenzyl-4-(2-methoxyethoxy)cyclohexan-1-amine

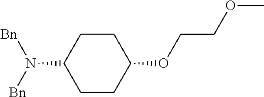

To a stirred solution of (1s,4s)-4-(dibenzylamino)cyclohexan-1-ol (1.5 g, 5.07 mmol) in DMPU (10 mL) at RT, sodium hydride (0.50 g, 12.69 mmol) was added under nitrogen atmosphere with continuous stirring (the mixture was became exothermic). Then 1-bromo-2-methoxyethane (1.76 g, 12.69 mmol) was added at RT over a period of 10 min. During addition of 1-bromo-2-methoxyethane foam formation was observed. After complete addition, the reaction mixture was stirred for 5 h at 50° C. After completion (monitored by TLC), the reaction mixture was cooled to RT and slowly poured into ice cold water (50 mL) with continuous stirring. The suspension was extracted with MTBE (300 mL). The organic layer was washed with water (2×200 mL), dried over anhydrous Na₂SO₄, filtered and the filtrate was concentrated under vacuum to get a brown oil residue. The crude residue was dissolved in HCl in dioxane (10 mL, 4 M), stirred for 10 min at RT and then concentrated. The resulting white solid was suspended in diethyl ether (20 mL) and the mixture stirred for 10 min. The mixture was filtered, the filtration cake was washed with diethyl ether (20 mL) and dried. The resulting hydrochloride salt was dissolved in aqueous NaOH solution (50 mL, 10% solution) and extracted with diethyl ether (100 mL). The combined organic layer was dried over Na₂SO₄, filtered, and then concentrated to get the title compound. Yield: 83% (1.5 g, pale brown oil). LCMS: (Method C) 354.3 (M+H)

### Step 3: (1s,4s)-4-(2-methoxyethoxy)cyclohexan-1-amine

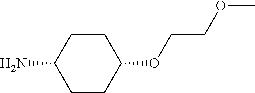

To a stirred solution of (1s,4s)-N,N-dibenzyl-4-(2-methoxyethoxy)cyclohexan-1-amine (1.5 g, 4.24 mmol) in absolute ethanol (10 mL) at RT, Pd(OH)₂ on carbon (0.20 g, 20% wt. basis) was added and the mixture was stirred for 16 h under hydrogen atmosphere at RT. After completion (monitored by TLC), the reaction mixture was filtered through Celite bed. The filtrate was evaporated under vacuum to afford the title compound which was used in the next step without further purification. Yield: 61% (0.450 g, colorless liquid). LCMS: (Method C) 174.2 (M+H)

### Step 4: N-((1s,4s)-4-(2-methoxyethoxy)cyclohexyl)-6-(thiazol-5-yl)-1H-indole-4-carboxamide

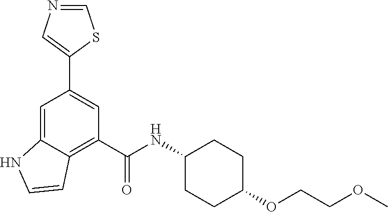 [SMILES: COCCO[C@H]1CC[C@@H](NC(=O)c2cc(-c3cncs3)cc3[nH]ccc23)CC1]

To a stirred solution of 6-(thiazol-5-yl)-1H-indole-4-carboxylic acid (0.1 g, 0.40 mmol) in DMF (3 mL) at 0° C., were added HATU (0.23 g, 0.61 mmol) followed by DIPEA (0.18 mL, 1.02 mmol) under nitrogen atmosphere. After stirring for 5 min at 0° C., (1s,4s)-4-(2-methoxyethoxy)cyclohexan-1-amine (0.10 g, 0.61 mmol) was added and the reaction mixture was stirred at RT for 16 h. After completion (starting material was consumed according to TLC), the reaction mixture was diluted with ice cold water (15 mL) and extracted with EtOAc (50 mL). The resulting organic layer was washed with water (2×25 mL), brine solution (2×50 mL), dried over anhydrous Na₂SO₄, filtered and the filtrate was concentrated under vacuum. The resulting crude residue was purified by reverse phase grace purification. The prep-fraction was concentrated under reduced pressure, the residue was diluted with 10% MeOH in DCM (10 mL) and washed with aq. NaHCO₃ solution and water. The organic layer was dried over anhydrous Na₂SO₄, filtered, concentrated under reduced pressure and lyophilized to get the title compound. Yield: 9% (14.90 mg, off white solid). ¹H NMR (400 MHz, DMSO-d₆): 11.38 (s, 1H), 9.05 (d, J=0.8 Hz, 1H), 8.32 (d, J=0.8 Hz, 1H), 8.24 (d, J=7.6 Hz, 1H), 7.75 (s, 1H), 7.72 (d, J=1.6 Hz, 1H), 7.50-7.49 (m, 1H), 6.85-6.84 (m, 1H), 3.93-3.87 (m, 1H), 3.53-3.50 (m, 3H), 3.48-3.45 (m, 2H), 3.30 (s, 3H), 1.90-1.86 (m, 2H), 1.76-1.60 (m, 4H), 1.55-1.48 (m, 2H). LCMS: (Method C) 400.2 (M+H)

### Example 49: methyl 5-(1H-imidazol-1-yl)-7-(((1r,4r)-4-(2-methoxyethoxy)cyclohexyl)carbamoyl)-1H-indole-3-carboxylate

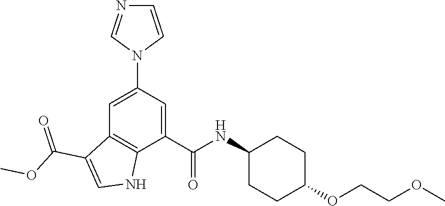

To a stirred solution of 3-cyano-5-(1H-imidazol-1-yl)-N-((1r,4r)-4-(2-methoxyethoxy)cyclohexyl)-1H-indole-7-carboxamide (200 mg, 0.490 mmol) in 10 mL methanol at RT Con HCl (2 mL) was added and heated to 100° C. for 96 h in sealed tube. After completion of reaction, the reaction mixture was concentrated under reduced pressure and the resulting crude was purified by Prep-HPLC (Method A). Prep fraction was collected, concentrated under reduced pressure. The resulting compound was dissolved in 10% MeOH in DCM (10 mL) and neutralized with sat.NaHCO₃ solution. Organic layer was dried over anhydrous Na₂SO₄, filtered, solvent removed and lyophilized to get off-white solid product. Yield: 25% (54.94 mg, Off-white solid). ¹HNMR (400 MHz, DMSO-d6): δ 12.02 (s, 1H), 8.54 (t, J=9.36 Hz, 1H), 8.22-8.21 (m, 2H), 8.05 (s, 1H), 7.99 (s, 1H), 7.74 (s, 1H), 7.15 (s, 1H), 3.88-3.84 (m, 4H), 3.57-3.54 (m, 2H), 3.45-3.43 (m, 2H), 3.36-3.35 (m, 4H), 2.09-2.04 (m, 2H), 1.99-1.93 (m, 2H), 1.44-1.41 (m, 2H), 1.31-1.25 (m, 2H), LCMS: (Method C) 441.3 (M+H)

### Example 50: N-((1r,4r)-4-(2-methoxyethoxy)cyclohexyl)-1-(oxetan-3-ylmethyl)-6-(thiazol-5-yl)-1H-indole-4-carboxamide

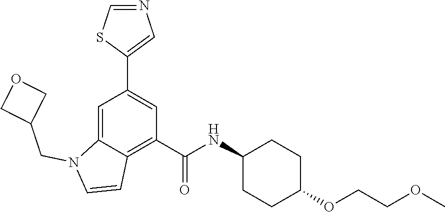 [SMILES: COCCO[C@H]1CC[C@H](NC(=O)c2cc(-c3cncs3)cc3c2ccn3CC2COC2)CC1]

To a stirred solution of N-((1r,4r)-4-(2-methoxyethoxy)cyclohexyl)-6-(thiazol-5-yl)-1H-indole-4-carboxamide (0.1 g. 0.25 mmol) in DMF (2 mL) at RT, was added cesium carbonate (244 mg, 0.75 mmol) and 3-(bromomethyl)oxetane (45.4 mg, 0.30 mmol) and the reaction mixture was stirred at RT for 16 h. After completion (monitored by TLC), the reaction mixture was quenched with water (10 mL) and extracted with EtOAc (3×10 mL). The combined organic layer was washed with water (5 mL), brine (5 mL), dried over anhydrous Na₂SO₄ and concentrated under vacuum. The resulting crude residue was purified by flash chromatography on Biotage Isolera (230-400 mesh silica gel, eluent: 0-5% methanol in DCM) to afford the title compound. Yield: 26% (30.81 mg, pale yellow solid). ¹H NMR (400 MHz, DMSO-d₆): δ 9.07 (d, J=0.4 Hz, 1H), 8.37 (d, J=0.8 Hz, 1H), 8.19 (d, J=7.6 Hz, 1H), 8.03 (s, 1H), 7.63 (d, J=1.2 Hz, 1H), 7.58 (d, J=3.2 Hz, 1H), 6.79-6.78 (m, 1H), 4.64-4.58 (m, 4H), 4.43 (t, J=6.0 Hz, 2H), 3.84-3.78 (m, 1H), 3.56-3.53 (m, 2H), 3.50-3.43 (m, 3H), 3.29-3.22 (s, 4H), 2.04-2.00 (m, 2H), 1.93-1.90 (m, 2H), 1.46-1.36 (m, 2H), 1.31-1.21 (m, 2H). LCMS: (Method A) 470.0 (M+H)

### Example 51: N-((1r,4r)-4-(2-methoxyethoxy)cyclohexyl)-1-methyl-6-(thiazol-5-yl)-1H-indole-4-carboxamide

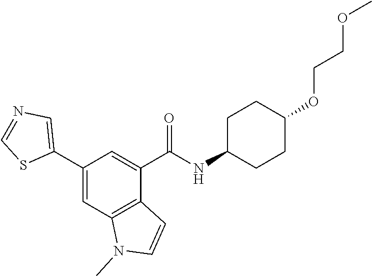 [SMILES: COCCO[C@H]1CC[C@H](NC(=O)c2cc(-c3cncs3)cc3c2ccn3C)CC1]

To a stirred solution of N-((1r,4r)-4-(2-methoxyethoxy)cyclohexyl)-6-(thiazol-5-yl)-1H-indole-4-carboxamide (0.1 g, 0.25 mmol) in DMF (2 mL), was added Cs₂CO₃ (179 mg, 0.55 mmol) and Mel (39 mg, 0.27 mmol) and the reaction mixture was stirred at RT for 16 h. The reaction mixture was quenched with water (10 mL) and extracted with EtOAc (3×20 mL). The combined organic layer was washed with water (5 mL), brine (5 mL), dried over anhydrous Na₂SO₄ and concentrated under vacuum. The resulting crude residue was purified by flash chromatography on Biotage Isolera (230-400 mesh silica gel, eluent: 0-5% methanol in DCM) to afford the title compound. Yield: 40% (20.7 mg, Pale yellow solid). ¹H NMR (400 MHz, DMSO-d₆): δ 9.07 (s, 1H), 8.36 (s, 1H), 8.19 (d, J=7.6 Hz, 1H), 7.90 (s, 1H), 7.67 (d, J=1.6 Hz, 1H), 7.47 (d, J=3.2 Hz, 1H), 6.80 (d, J=2.8 Hz, 1H), 3.88-3.80 (m, 4H), 3.56-3.53 (m, 2H), 3.45-3.43 (m, 2H), 3.29-3.22 (m, 4H), 2.05-2.02 (m, 2H), 1.94-1.91 (m, 2H), 1.47-1.32 (m, 2H), 1.28-1.21 (m, 2H). LCMS: (Method A) 414.0 (M+H)

### Example 52: 1-isopropyl-N-((1r,4r)-4-(2-methoxyethoxy)cyclohexyl)-6-(thiazol-5-yl)-1H-indole-4-carboxamide

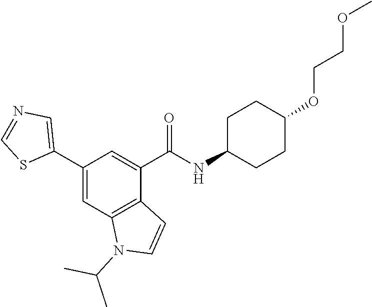 [SMILES: COCCO[C@H]1CC[C@H](NC(=O)c2cc(-c3cncs3)cc3c2ccn3C(C)C)CC1]

To a stirred solution of N-((1r,4r)-4-(2-methoxyethoxy)cyclohexyl)-6-(thiazol-5-yl)-1H-indole-4-carboxamide (0.1 g, 0.25 mmol) in DMF (2 mL), were added Cs₂CO₃ (244 mg, 0.75 mmol) and isopropyl iodide (51 mg, 0.30 mmol) at RT and the reaction mixture was stirred at RT for 16 h. The reaction mixture was quenched with water (10 mL) and extracted with EtOAc (3×20 mL). The combined organic layer was washed with water (10 mL), brine (10 mL), dried over anhydrous Na₂SO₄ and concentrated under vacuum. The resulting crude residue was purified by Prep-HPLC (Method A) followed by flash chromatography on Biotage Isolera (230-400 mesh silica gel, eluent: 0-5% methanol in DCM) to afford the title compound. Yield: 2.5% (2.75 mg, pale yellow solid). ¹H NMR (400 MHz, CD₃OD): δ 8.97 (s, 1H), 8.27 (s, 1H), 7.91 (s, 1H), 7.67 (d, J=1.6 Hz, 1H), 7.58 (d, J=3.2 Hz, 1H), 6.85 (d, J=2.8 Hz, 1H), 4.01-3.96 (m, 1H), 3.68-3.65 (m, 2H), 3.57-3.54 (m, 2H), 3.41-3.30 (m, 5H), 2.18-2.10 (m, 4H), 1.58-1.55 (m, 6H), 1.53-1.36 (m, 4H). LCMS: (Method C) 442.3 (M+H)

### Example 53: 1-(2-hydroxy-2-methylpropyl)-N-((1r,4r)-4-(2-methoxyethoxy)cyclohexyl)-6-(thiazol-5-yl)-1H-indole-4-carboxamide

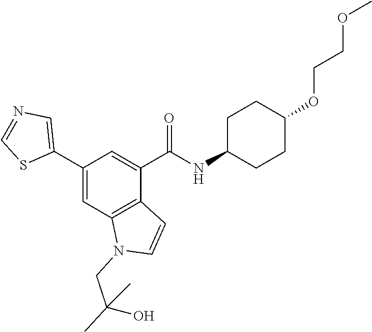 [SMILES: COCCO[C@H]1CC[C@H](NC(=O)c2cc(-c3cncs3)cc3c2ccn3CC(C)(C)O)CC1]

To a stirred solution of N-((1r,4r)-4-(2-methoxyethoxy)cyclohexyl)-6-(thiazol-5-yl)-1H-indole-4-carboxamide (0.1 g, 0.25 mmol) in DMF (2 mL), were added CsCO₃ (244 mg, 0.75 mmol) and 2,2-dimethyloxirane (21.6 mg, 0.30 mmol) and the reaction mixture was stirred at RT for 16 h. The reaction mixture was quenched with water (10 mL) and extracted with EtOAc (3×20 mL). The combined organic layer was washed with water (10 mL), brine (10 mL), dried over anhydrous Na₂SO₄ and concentrated under vacuum. The resulting crude residue was purified by Prep-HPLC (Method A) to afford the title compound. Yield: 4.4% (5.23 mg, white solid). ¹H NMR (400 MHz, DMSO-d₆): δ 9.06 (d, J=0.4 Hz, 1H), 8.33 (d, J=0.4 Hz, 1H), 8.19 (d, J=8.0 Hz, 1H), 8.02 (s, 1H), 7.61 (d, J=1.2 Hz, 1H), 7.47 (d, J=2.8 Hz, 1H), 6.80 (d, J=2.8 Hz, 1H), 4.71 (s, 1H), 4.17 (s, 2H), 3.84-3.80 (m, 1H), 3.56-3.54 (m, 2H), 3.45-3.34 (m, 2H), 3.28-3.22 (s, 4H), 2.05-2.02 (m, 2H), 1.94-1.91 (m, 2H), 1.46-1.37 (m, 2H), 1.30-1.22 (m, 2H), 1.11 (s, 6H). LCMS: (Method C) 472.2 (M+H)

### Example 54: 5-(1H-imidazol-1-yl)-N-((1r,4r)-4-(2-methoxyethoxy)cyclohexyl)-1-methyl-1H-indole-7-carboxamide

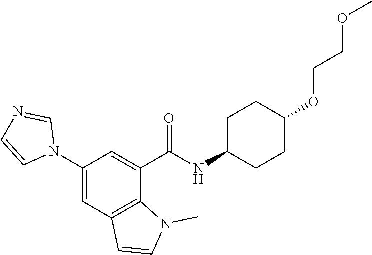 [SMILES: COCCO[C@H]1CC[C@H](NC(=O)c2cc(-n3ccnc3)cc3ccn(C)c23)CC1]

To a stirred solution of 5-(1H-imidazol-1-yl)-N-((1r,4r)-4-(2-methoxyethoxy)cyclohexyl)-1H-indole-7-carboxamide (100 mg, 0.26 mmol) in DMF (3.0 mL) at 0° C., Cs₂CO₃ (187 mg, 0.57 mmol) was added and the reaction mixture was stirred at 0° C. for 10 min. Then Mel (41 mg, 0.29 mmol) was added and the reaction mixture was stirred at RT for 6 h. The reaction was monitored by TLC. After completion, the reaction mixture was diluted with water (10 mL) and the resulting suspension was extracted with DCM (3×15 mL). The combined organic layer was washed with water (20 mL), brine (20 mL), dried over anhydrous Na₂SO₄ and concentrated under vacuum. The resulting crude residue was purified by Prep-HPLC (Method A). The prep fraction was concentered under vacuum, and the residue was dissolved in DCM and neutralized with 10% aq. NaHCO₃. The organic phase was washed with water, brine, dried over anhydrous Na₂SO₄ and evaporated to afford the title compound. Yield: 29% (30 mg, off white solid). ¹H NMR (400 MHz, DMSO-d₆): δ 8.55 (d, J=7.7 Hz, 1H), 8.16 (s, 1H), 7.83 (d, J=2.1 Hz, 1H), 7.70 (s, 1H), 7.46 (d, J=3.0 Hz, 1H), 7.28 (d, J=2.0 Hz, 1H), 7.10 (s, 1H), 6.55 (d, J=3.0 Hz, 1H), 3.83-3.78 (m, 4H), 3.55-3.52 (m, 2H), 3.44-3.41 (m, 2H), 3.28-3.21 (m, 4H), 2.03-1.95 (m, 4H), 1.39-1.22 (m, 4H). LCMS: (Method C) 397.1 (M+H)

### Example 55: 7-fluoro-N-((1r,4r)-4-(2-methoxyethoxy)cyclohexyl)-6-(thiazol-5-yl)-1H-indole-4-carboxamide

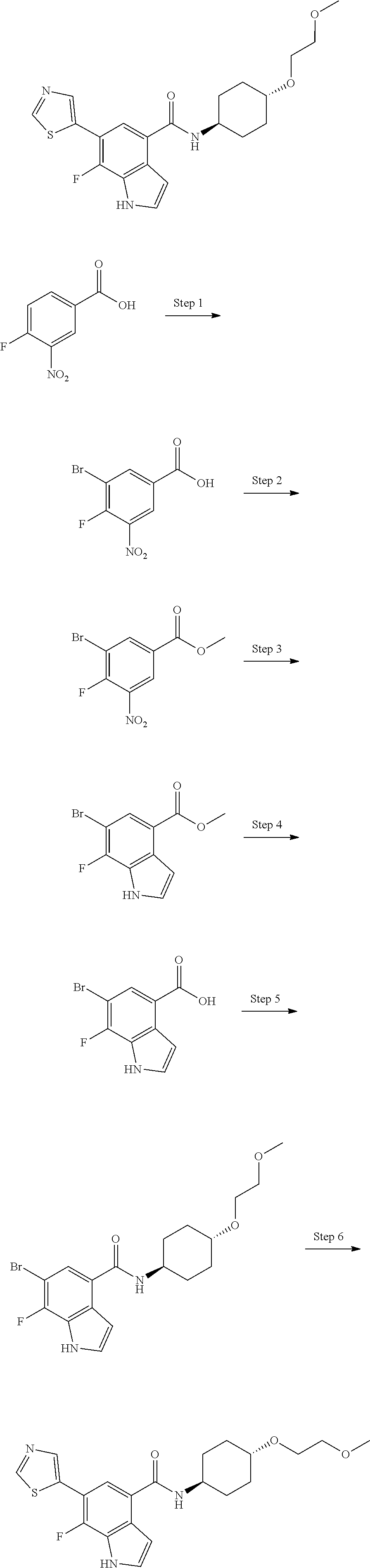

### Step 1: 3-bromo-4-fluoro-5-nitrobenzoic acid

To a stirred solution of 4-fluoro-3-nitrobenzoic acid (4 g, 21.60 mmol) in a Conc. H₂SO₄ (5 mL) at RT, NBS (3.84 g, 21.60 mmol) was added and the reaction mixture was heated at 70° C. for 7 h. After completion of reaction, the reaction mixture was poured into ice cold water (20 mL) and stirred it for 5 min. Solid was filtered, washed with water (2×25 mL) and dried to get title product. Yield: 70% (4.01 g, off white solid). ¹H NMR (400 MHz, DMSO-d₆): δ 14.01 (s, 1H), 8.54-8.51 (m, 2H). LCMS: (Method C) 261.9 (M+H)

### Step 2: methyl 3-bromo-4-fluoro-5-nitrobenzoate

To a stirred solution of 3-bromo-4-fluoro-5-nitrobenzoic acid (2 g, 7.57 mmol) in a MeOH (20 mL) at 0° C., SOCl₂ (1.09 mL, 15.15 mmol) was added and the reaction mixture was stirred at 70° C. for 16 h. The reaction mixture was monitored by TLC. After complete consumption of the starting material the reaction mixture was concentrated under vacuum. The residue was dissolved in EtOAc (500 mL), washed with 10% NaHCO₃ solution (200 mL), brine solution (200 mL), dried over anhydrous Na₂SO₄ and concentrated under vacuum to get title compound. Yield: 71.2% (1.5 g, off white solid). ¹H NMR (400 MHz, DMSO-d₆): 8.57-8.54 (m, 2H), 3.99 (s, 3H). LCMS: (Method C) 278.9 (M+H)

### Step 3: methyl 6-bromo-7-fluoro-1H-indole-4-carboxylate

To a stirred solution of methyl 3-bromo-4-fluoro-5-nitrobenzoate (700 mg, 2.51 mmol) in a THF (10 mL) at −78° C., vinyl magnesium bromide solution (1 M in THF) (10.07 mL, 10.07 mmol) was added and the reaction mixture was stirred at −78° C. for 4 h. The reaction mixture was monitored by TLC. After completion of reaction, the reaction mixture was warmed to RT and quenched with addition of saturated ammonium chloride solution (100 mL). The resulting suspension was extracted with EtOAc (200 mL), washed with brine (100 mL), water (100 mL), dried over anhydrous Na₂SO₄ and concentrated under vacuum. The resulting crude residue was purified by flash chromatography on Biotage Isolera (100-200 mesh silica gel, 5%-10% EtOAc in Petroleum ether) to get title compound. Yield: 18.2% (250 mg, yellow solid). ¹H NMR (400 MHz, DMSO-d₆): δ 12.26 (s, 1H), 7.86 (d, J=6.0 Hz, 1H), 7.66-7.65 (m, 1H), 7.03-7.00 (m, 1H), 3.91 (s, 3H). LCMS: (Method C) 271.8 (M−H)

### Step 4: 6-bromo-7-fluoro-1H-indole-4-carboxylic acid

To a stirred solution of methyl 6-bromo-7-fluoro-1H-indole-4-carboxylate (250 mg, 0.91 mmol) in a mixture of THF (3 mL) and water (0.8 mL) at RT, LiOH.H₂O (96.17 mg, 2.29 mmol) was added and stirred at RT for 16 h. After completion of reaction, the reaction mixture was evaporated under vacuum and residue was acidified with 1.5N HCl solution. The resulting suspension was extracted with EtOAc (100 mL), washed with brine solution (25 mL), water (25 mL), dried over anhydrous Na₂SO₄, filtered and the filtrate was concentrated under vacuum to get title compound. Yield: 88.7% (210 mg, yellow solid). ¹HNMR (400 MHz, DMSO-d₆): 7.84 (d, J=6.0 Hz, 1H), 7.62-7.60 (t, J=2.8 Hz, 1H), 7.03-7.01 (m, 1H), LCMS: (Method C) 256.0 (M−H)

### Step 5: 6-bromo-7-fluoro-N-((1r,4r)-4-(2-methoxyethoxy)cyclohexyl)-1H-indole-4-carboxamide

To a stirred solution of 6-bromo-7-fluoro-1H-indole-4-carboxylic acid (210 mg, 0.81 mmol) in DMF (5 mL) at 0° C. EDC.HCl (233 mg, 1.21 mmol, HOBt (164 mg, 1.21 mmol) and TEA (0.29 mL, 2.03 mmol) were added under nitrogen atmosphere. Then after 5 min (1r,4r)-4-(2-methoxyethoxy)cyclohexan-1-amine (169 mg, 0.97 mmol) in DMF was added at same temperature. Further reaction was stirred for another 16 h at RT. After completion of reaction, the reaction mixture was quenched with ice cold water (50 mL) and then extracted with EtOAc (100 mL). The resulting organic solution was washed with brine solution (50 mL), water (50 mL), dried over Na₂SO₄, and concentrated under vacuum to get crude product. The resulting crude residue was purified by flash chromatography on Biotage Isolera (100-200 mesh silica gel, 50%-80% EtOAc in petroleum ether) to get title compound. Yield: 68.3% (230 mg, yellow solid). ¹H NMR (400 MHz, DMSO-d₆): δ 11.99 (s, 1H), 8.18 (d, J=7.6 Hz, 1H), 7.62-7.60 (m, 1H), 7.55-7.51 (m, 1H), 6.91 (s, 1H), 3.75-3.70 (m, 1H), 3.53-3.50 (m, 2H), 3.42-3.40 (m, 2H), 3.25-3.16 (m, 4H), 2.20-1.86 (m, 4H), 1.39-1.22 (m, 4H). LCMS: (Method C) 415.1 (M+H)

### Step 6: 7-fluoro-N-((1r,4r)-4-(2-methoxyethoxy)cyclohexyl)-6-(thiazol-5-yl)-1H-indole-4-carboxamide

To a stirred solution of 6-bromo-7-fluoro-N-((1r,4r)-4-(2-methoxyethoxy)cyclohexyl)-1H-indole-4-carboxamide (102 mg, 0.24 mmol) and 5-(4,4,5,5-tetramethyl-1,3,2-dioxaborolan-2-yl)thiazole (78.1 mg, 0.37 mmol) in a mixture of 1,4 Dioxane (2 mL) and water (0.5 mL) nitrogen gas was purged for 5 min before the addition of K₂CO₃ (84.8 mg, 0.61 mmol), CuI (4.6 mg, 0.02 mmol) Pd(dppf)Cl₂.DCM (20 mg, 0.02 mmol) at RT. Then the reaction mixture was heated at 100° C. for 16 h. After completion of reaction, the reaction mixture was filtered through Celite and washed with EtOAc (100 mL). The filtrate was dried over anhydrous Na₂SO₄, filtered and the filtrate was concentrated under vacuum. The resulting crude residue was purified by flash chromatography on Biotage Isolera (100-200 mesh silica gel, 50%-70% EtOAc in Petroleum ether) to get crude compound. The resulting product was further purified by Prep-HPLC (Method B). The prep-fraction was collected, concentrated under reduced pressure, dissolved in 10% MeOH/DCM (10 mL) and washed with brine solution (5 mL) followed by water (10 mL). The organic layer was dried over anhydrous Na₂SO₄, filtered, concentrated under reduced pressure and lyophilized to get the title compound. Yield: 5% (5.32 mg, off white solid). ¹HNMR (400 MHz, DMSO-d₆): 12.05 (s, 1H), 9.18 (s, 1H), 8.39 (s, 1H), 8.19 (d, J=8.0 Hz, 1H), 7.74 (d, J=6.4 Hz, 1H), 7.56 (d, J=3.2 Hz, 1H), 6.92-6.91 (m, 1H), 3.83-3.79 (m, 1H), 3.56-3.50 (m, 2H), 3.45-3.40 (m, 2H), 3.29-3.20 (m, 4H), 2.05-2.00 (m, 2H), 1.94-1.91 (m, 2H), 1.45-1.37 (m, 2H), 1.30-1.22 (m, 2H). LCMS: (Method C) 418.1 (M+H)

### Example 56: 3-cyano-6-(1H-imidazol-1-yl)-N-((1r,4r)-4-(2-methoxyethoxy)cyclohexyl)-1H-indole-4-carboxamide

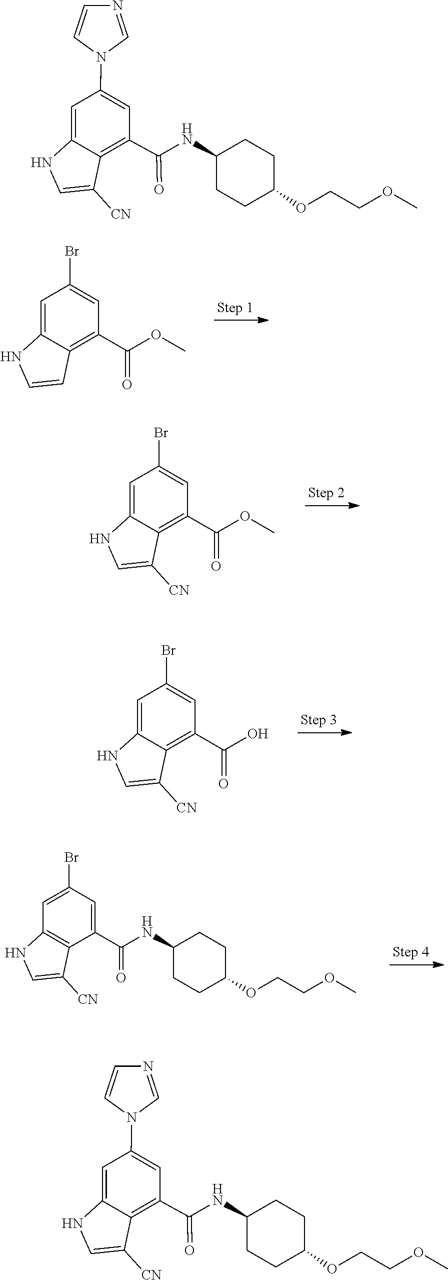 [SMILES: COCCO[C@H]1CC[C@H](NC(=O)c2cc(-n3ccnc3)cc3[nH]cc(C#N)c23)CC1 ; COC(=O)c1cc(Br)cc2[nH]ccc12 ; COC(=O)c1cc(Br)cc2[nH]cc(C#N)c12 ; N#Cc1c[nH]c2cc(Br)cc(C(=O)O)c12 ; COCCO[C@H]1CC[C@H](NC(=O)c2cc(Br)cc3[nH]cc(C#N)c23)CC1 ; COCCO[C@H]1CC[C@H](NC(=O)c2cc(-n3ccnc3)cc3[nH]cc(C#N)c23)CC1]

### Step 1: methyl 6-bromo-3-cyano-1H-indole-4-carboxylate

To a stirred solution of methyl 6-bromo-1H-indole-4-carboxylate (1.2 g, 47.06 mmol) in ACN (25 mL) chlorosulfonyl isocyanate (665 mg, 47.06 mmol) was added slowly over 2 min at 0° C. After complete addition, the reaction mixture was stirred for 45 min at 0° C., then NEt₃ (475 mg, 47.06 mmol) was added slowly over 2 min. Then further reaction mixture was stirred at RT for 16 h. After completion of reaction, the reaction mixture was quenched with water (20 mL), stirred for 15 min and extracted with EtOAc (3×50 mL). The combined organic layer was washed with water (25 mL), brine (25 mL), dried over anhydrous Na₂SO₄ and concentrated under vacuum. The crude residue was purified by flash chromatography on Biotage Isolera (silica gel: 100-200 mesh, eluent: 0-50% EtOAc in petroleum ether). Yield: 61% (0.8 g, yellow solid). ¹HNMR 400 MHz, DMSO-d6: δ 12.70 (s, 1H), 8.49 (d, J=3.2 Hz, 1H), 8.01 (d, J=2.0 Hz, 1H), 7.87 (d, J=1.6 Hz, 1H), 3.90 (s, 3H). LCMS: (Method C) 281.0 (M+H)

### Step 2: 6-bromo-3-cyano-1H-indole-4-carboxylic acid

To a stirred solution of methyl 6-bromo-3-cyano-1H-indole-4-carboxylate (0.8 g, 2.86 mmol) in THF:MeOH:H₂O (1:1:1, 30 mL) was added NaOH (344 mg, 8.60 mmol) at RT and the mixture was stirred for 16 h at same temp. After completion of the reaction, the reaction mixture was concentrated. The resulting crude residue was dissolved in water (15 ml) and it was acidified using HCl (2N). The resulting solid obtained was collected using filtration. Yield: 57% (430 mg, white solid). ¹HNMR (400 MHz, DMSO-d6): δ 13.46 (s, 1H), 12.62 (s, 1H), 8.45 (d, J=2.8 Hz, 1H), 7.97 (d, J=2.0 Hz, 1H), 7.83 (d, J=2.0 Hz, 1H). LCMS: (Method C) 262.9 (M−H)

### Step 3: 6-bromo-3-cyano-N-((1r,4r)-4-(2-methoxyethoxy)cyclohexyl)-1H-indole-4-carboxamide

To a stirred solution of 6-bromo-3-cyano-1H-indole-4-carboxylic acid (200 mg, 0.75 mmol) in THF:DCM (1:1, 4 mL), were added EDC.HCl (187 mg, 0.97 mmol), HOBt (152 mg, 1.12 mmol) and NEt₃ (0.3 mL, 2.25 mmol) followed by (1r,4r)-4-(2-methoxyethoxy)cyclohexan-1-amine (156 mg, 0.9 mmol) at RT and stirred for 12 h. After completion of reaction, the reaction mixture was concentrated and to the resulting crude water (10 mL) and extracted with DCM (3×20 mL). The combined organic layer was washed with brine (10 mL), dried over anhydrous Na₂SO₄ and concentrated under vacuum. The resulting crude residue was purified by flash chromatography on Biotage Isolera (230-400 mesh silica gel, 0-5% methanol in DCM) to afford the title compound.

Yield: 35% (110 mg, yellow solid). ¹HNMR 400 MHz, DMSO-d6: δ 12.5 (s, 1H), 8.41 (d, J=7.6 Hz, 1H), 8.34 (s, 1H), 7.81 (d, J=1.6 Hz, 1H), 7.41 (d, J=1.6 Hz, 1H), 3.74-3.73 (m, 1H), 3.55-3.52 (m, 2H), 3.44-3.41 (m, 2H), 3.28-3.22 (m, 4H), 2.02-1.98 (m, 4H), 1.36-1.23 (m, 4H). LCMS: (Method C) 420.0 (M−H)

### Step 4: 3-cyano-6-(1H-imidazol-1-yl)-N-((1r,4r)-4-(2-methoxyethoxy)cyclohexyl)-1H-indole-4-carboxamide

To a stirred solution of 6-bromo-3-cyano-N-((1r,4r)-4-(2-methoxyethoxy)cyclohexyl)-1H-indole-4-carboxamide (110 mg, 0.17 mmol), Imidazole (107 mg, 1.56 mmol) and K₂CO₃ (145 mg, 1.04 mmol) in DMSO (1.5 mL) was purged with nitrogen gas for 10 min and then charged with L-proline (15 mg, 0.13 mmol) and CuI (24 mg, 0.13 mmol) at RT. Then the reaction mixture was stirred at 95° C. for 16 h. After completion of the reaction, the reaction mixture was filtered through Celite, filtrate was concentrated, and the resulting crude residue was purified by Prep-HPLC (Method A) to afford the title compound. Yield: 22% (22.8 mg, off white solid). ¹HNMR (400 MHz, DMSO-d6): δ 12.54 (s, 1H), 8.40-8.38 (m, 2H), 8.29 (s, 1H), 7.81-7.79 (m, 2H), 7.55 (d, J=2.0 Hz, 1H), 7.14 (s, 1H), 3.82-3.76 (m, 1H), 3.56-3.53 (m, 2H), 3.44-3.43 (m, 2H), 3.26-3.24 (m, 4H), 2.04-2.01 (m, 4H), 1.41-1.22 (m, 4H). LCMS: (Method B) 407.90 (M+H)

### Example 57: N-((1r,4r)-4-(2-methoxyethoxy)cyclohexyl)-3-methyl-6-(thiazol-5-yl)-1H-indole-4-carboxamide

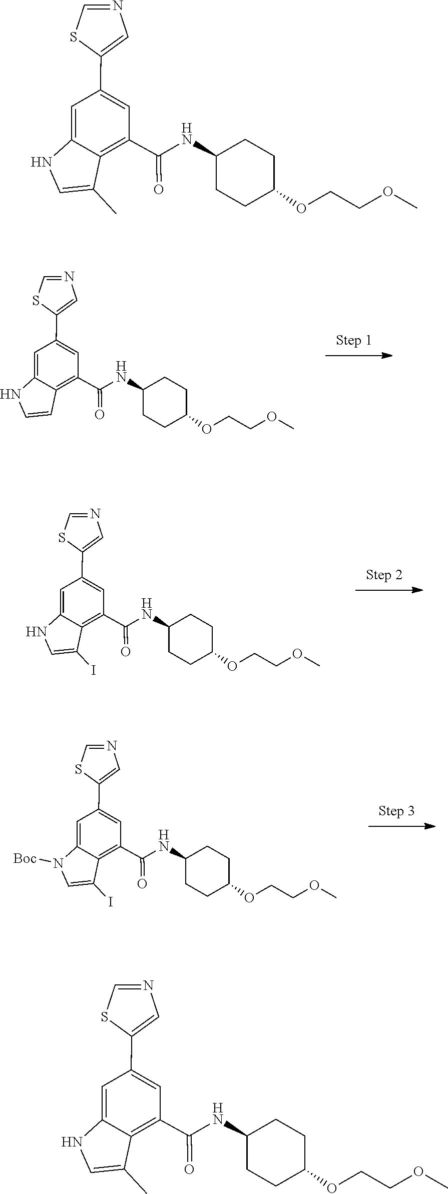 [SMILES: COCCO[C@H]1CC[C@H](NC(=O)c2cc(-c3cncs3)cc3[nH]cc(C)c23)CC1 ; COCCO[C@H]1CC[C@H](NC(=O)c2cc(-c3cncs3)cc3[nH]ccc23)CC1 ; COCCO[C@H]1CC[C@H](NC(=O)c2cc(-c3cncs3)cc3[nH]cc(I)c23)CC1 ; COCCO[C@H]1CC[C@H](NC(=O)c2cc(-c3cncs3)cc3c2c(I)cn3C(=O)OC(C)(C)C)CC1 ; COCCO[C@H]1CC[C@H](NC(=O)c2cc(-c3cncs3)cc3[nH]cc(C)c23)CC1]

### Step 1: 3-iodo-N-((1r,4r)-4-(2-methoxyethoxy)cyclohexyl)-6-(thiazol-5-yl)-1H-indole-4-carboxamide

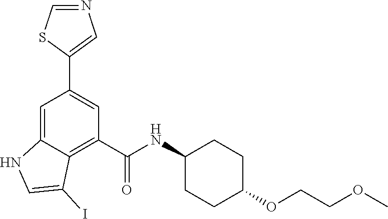 [SMILES: COCCO[C@H]1CC[C@H](NC(=O)c2cc(-c3cncs3)cc3[nH]cc(I)c23)CC1]

To a stirred solution of N-((1r,4r)-4-(2-methoxyethoxy)cyclohexyl)-6-(thiazol-5-yl)-1H-indole-4-carboxamide (0.2 g, 0.50 mmol) in DMF (3 mL), was added KOH (84 mg, 1.50 mmol) and iodine (127 mg, 0.50 mmol) at RT and the reaction mixture was stirred at RT for 1 h. After completion, the reaction mixture was quenched with aq. Na₂S₂O₃ solution. The resulting solid was collected using filtration and dried to get the title compound. Yield: 72% (190 mg, white solid). ¹H NMR (400 MHz, DMSO-d₆): δ 11.80 (s, 1H), 9.06 (s, 1H), 8.31 (s, 1H), 8.27 (d, J=7.6 Hz, 1H), 7.72 (d, J=1.6 Hz, 1H), 7.66 (s, 1H), 7.24 (d, J=1.6 Hz, 1H), 3.84-3.79 (m, 1H), 3.55-3.52 (m, 2H), 3.44-3.41 (m, 2H), 3.28-3.22 (m, 4H), 2.11-1.98 (m, 4H), 1.40-1.21 (m, 4H). LCMS: (Method C) 526.0 (M+H)

### Step 2: tert-butyl 3-iodo-4-(((1r,4r)-4-(2-methoxyethoxy)cyclohexyl)carbamoyl)-6-(thiazol-5-yl)-1H-indole-1-carboxylate

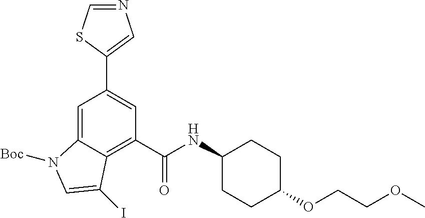 [SMILES: COCCO[C@H]1CC[C@H](NC(=O)c2cc(-c3cncs3)cc3c2c(I)cn3C(=O)OC(C)(C)C)CC1]

To a stirred solution of 3-iodo-N-((1r,4r)-4-(2-methoxyethoxy)cyclohexyl)-6-(thiazol-5-yl)-1H-indole-4-carboxamide (180 mg, 0.34 mmol) and DIPEA (132 mg, 1.03 mmol) in THF (4 mL) were added DMAP (21 mg, 0.17 mmol) and Boc anhydride (112 mg, 0.51 mmol) at RT and the reaction mixture was stirred at RT for 16 h. After completion, the reaction mixture was diluted with water (10 mL) and extracted with EtOAc (3×15 mL). The combined organic layer was washed with water (10 mL), brine (10 mL), dried over anhydrous Na₂SO₄ and filtered. The filtrate was concentrated under vacuum, and the crude residue was purified by flash chromatography on Biotage Isolera (230-400 mesh silica gel, eluent: 0-3% MeOH in DCM as gradient) to afford the title compound. Yield: 67% (145 mg, white solid). ¹H NMR (400 MHz, DMSO-d₆): δ 9.12 (s, 1H), 8.43-8.39 (m, 3H), 7.95-7.93 (m, 1H), 7.53 (s, 1H), 3.84-3.71 (m, 1H), 3.55-3.52 (m, 2H), 3.44-3.41 (m, 2H), 3.29-3.23 (m, 4H), 2.09-1.99 (m, 4H), 1.66 (s, 9H), 1.37-1.18 (m, 4H). LCMS: (Method C) 626.1 (M+H)

### Step 3: N-((1r,4r)-4-(2-methoxyethoxy)cyclohexyl)-3-methyl-6-(thiazol-5-yl)-1H-indole-4-carboxamide

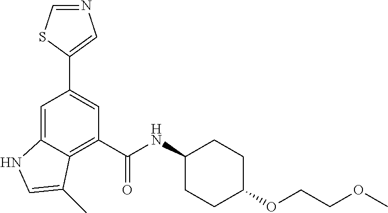 [SMILES: COCCO[C@H]1CC[C@H](NC(=O)c2cc(-c3cncs3)cc3[nH]cc(C)c23)CC1]

A stirred solution of tert-butyl 3-iodo-4-(((1r,4r)-4-(2-methoxyethoxy)cyclohexyl)carbamoyl)-6-(thiazol-5-yl)-1H-indole-1-carboxylate (110 mg, 0.17 mmol) in DMF (2 mL) was purged with nitrogen gas for 10 min at RT. Then tetramethyltin (95 mg, 0.53 mmol) and Pd(dppf)Cl₂.DCM complex (14 mg, 0.02 mmol) were added at RT and the reaction mixture was stirred at 110° C. for 16 h. After completion, the reaction mixture was diluted with water (10 mL) and extracted with EtOAc (3×15 mL). The combined organic layer was washed with water (10 mL), brine (10 mL), dried over anhydrous Na₂SO₄ and filtered. The filtrate was concentrated under vacuum, and the crude residue was purified by Prep-HPLC (Method A) to afford the title compound. Yield: 15% (11 mg, white solid). Yield: 15% (11 mg, white solid). ¹H NMR (400 MHz, DMSO-d₆): δ 11.1 (s, 1H), 9.03 (d, J=0.4 Hz, 1H), 8.30-8.26 (m, 2H), 7.64 (d, J=1.2 Hz, 1H), 7.24-7.21 (m, 2H), 3.82-3.75 (m, 1H), 3.55-3.52 (m, 2H), 3.44-3.41 (m, 2H), 3.27-3.20 (m, 4H), 2.20 (s, 3H), 2.05-1.94 (m, 4H), 1.36-1.24 (m, 4H). LCMS: (Method C) 414.2 (M+H)

### Example 58: N-((1r,4r)-4-(2-methoxyethoxy)cyclohexyl)-6-(thiazol-5-yl)-1H-pyrrolo[2,3-b]pyridine-4-carboxamide

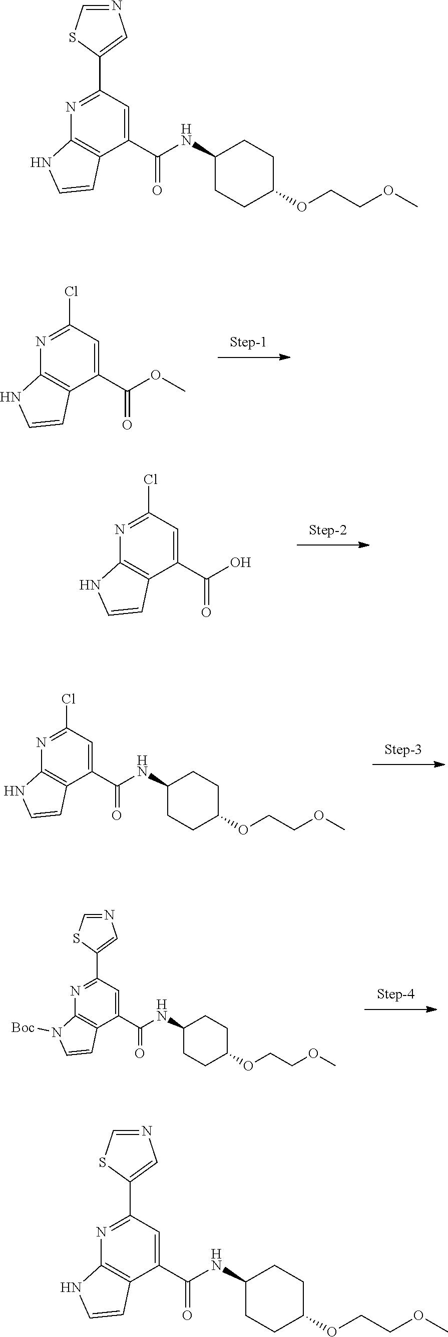 [SMILES: COCCO[C@H]1CC[C@H](NC(=O)c2cc(-c3cncs3)nc3[nH]ccc23)CC1 ; COC(=O)c1cc(Cl)nc2[nH]ccc12 ; O=C(O)c1cc(Cl)nc2[nH]ccc12 ; COCCO[C@H]1CC[C@H](NC(=O)c2cc(Cl)nc3[nH]ccc23)CC1 ; COCCO[C@H]1CC[C@H](NC(=O)c2cc(-c3cncs3)nc3c2ccn3C(=O)OC(C)(C)C)CC1 ; COCCO[C@H]1CC[C@H](NC(=O)c2cc(-c3cncs3)nc3[nH]ccc23)CC1]

### Step 1: 6-chloro-1H-pyrrolo[2,3-b]pyridine-4-carboxylic acid

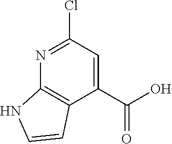 [SMILES: O=C(O)c1cc(Cl)nc2[nH]ccc12]

To a stirred solution of methyl 6-chloro-1H-pyrrolo[2,3-b]pyridine-4-carboxylate (0.4 g, 1.89 mmol) in a mixture of THF (8 mL) and water (2 mL) at RT, was added NaOH (0.227 g, 5.69 mmol) and the reaction mixture was stirred at RT for 16 h. After completion (monitored by TLC), the reaction mixture was concentrated under vacuum and acidified with aq. HCl (1.5 N) to adjust to pH 2. The precipitated solid was filtered and dried under vacuum to get the title compound. Yield: 80% (0.3 g, off white solid). ¹H NMR (400 MHz, DMSO-d₆): δ 7.71 (s, 1H), 7.53 (d, J=1.2 Hz, 1H), 6.87 (d, J=1.6 Hz, 1H). LCMS: (Method C) 197.1 (M+H)

### Step 2: 6-chloro-N-((1r,4r)-4-(2-methoxyethoxy)cyclohexyl)-1H-pyrrolo[2,3-b]pyridine-4-carboxamide

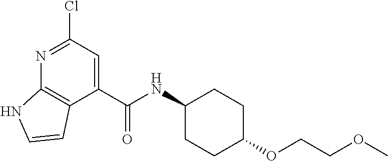 [SMILES: COCCO[C@H]1CC[C@H](NC(=O)c2cc(Cl)nc3[nH]ccc23)CC1]

To a stirred solution of 6-chloro-1H-pyrrolo[2,3-b]pyridine-4-carboxylic acid (0.3 g, 1.52 mmol) in DCM (10 mL) at RT were added TEA (0.64 mL, 4.56 mmol), EDC-HCl (0.437 g, 2.28 mmol) and HOBt (0.309 g, 2.28 mmol). After stirring for 5 min at RT, (1r,4r)-4-(2-methoxyethoxy)cyclohexan-1-amine (0.396 g, 2.28 mmol) was added and the reaction mixture was stirred at RT for 16 h. After completion (monitored by TLC), the reaction mixture was diluted with water (10 mL) and the resulting suspension was extracted with DCM (2×10 mL). The combined organic layer was washed with water (10 mL), brine (10 mL), dried over anhydrous Na₂SO₄, and concentrated under vacuum. The resulting crude residue was purified by flash chromatography on Biotage Isolera (60-120 mesh silica gel, eluent: 3% MeOH in DCM) to afford the title compound. Yield: 37% (0.2 g, off white solid). LCMS: (Method C) 352.2 (M+H)

### Step 3: tert-butyl 6-chloro-4-(((1r,4r)-4-(2-methoxyethoxy)cyclohexyl)carbamoyl)-1H-pyrrolo[2,3-b]pyridine-1-carboxylate

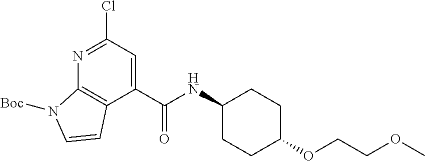 [SMILES: COCCO[C@H]1CC[C@H](NC(=O)c2cc(Cl)nc3c2ccn3C(=O)OC(C)(C)C)CC1]

To a stirred solution of 6-chloro-N-((1r,4r)-4-(2-methoxyethoxy)cyclohexyl)-1H-pyrrolo[2,3-b]pyridine-4-carboxamide (0.175 g, 0.50 mmol) and DMAP (6.9 mg, 0.06 mmol) in THF (6 mL) at RT was added Boc-anhydride (0.162 g, 0.75 mmol) and the reaction mixture was stirred at RT for 4 h. After completion (monitored by TLC), the reaction mixture was diluted with water (10 mL) and extracted with EtOAc (2×10 mL). The combined organic layer was washed with water (10 mL), brine (10 mL), dried over anhydrous Na₂SO₄ and concentrated under vacuum to afford the title compound. Yield: 77% (0.17 g, off white solid). LCMS: (Method D) 452.2 (M+H)

### Step 4: N-((1r,4r)-4-(2-methoxyethoxy)cyclohexyl)-6-(thiazol-5-yl)-1H-pyrrolo[2,3-b]pyridine-4-carboxamide

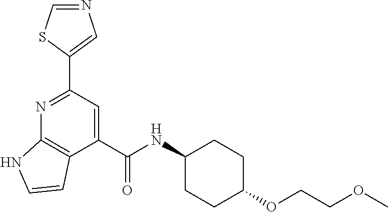 [SMILES: COCCO[C@H]1CC[C@H](NC(=O)c2cc(-c3cncs3)nc3[nH]ccc23)CC1]

To a stirred solution of tert-butyl 6-chloro-4-(((1r,4r)-4-(2-methoxyethoxy)cyclohexyl) carbamoyl)-1H-pyrrolo[2,3-b]pyridine-1-carboxylate (150 mg, 0.33 mmol) in a mixture of ethanol (4.5 mL) and water (0.5 mL) at RT, 5-(4,4,5,5-tetramethyl-1,3,2-dioxaborolan-2-yl)thiazole (105 mg, 0.49 mmol), CuI (3.1 mg, 0.02 mmol) and potassium carbonate (137 mg, 0.99 mmol) were added and the resulting mixture was purged with nitrogen gas for 2 min at RT. Then Pd(dppf)Cl₂.DCM (13.5 mg, 0.016 mmol) was added and the reaction mixture was heated at 100° C. for 16 h. After completion, the reaction mixture was filtered through Celite, and the filtration bed was washed with 10% MeOH in DCM (25 mL). The combined filtrate was concentrated vacuum and the resulting crude residue was purified by Prep-HPLC (Method A). The prep-fraction was concentrated, the residue was neutralized with sat. aq. NaHCO₃ solution and extracted with 10% MeOH in DCM. The combined organic layer was washed with brine, dried over anhydrous Na₂SO₄, filtered, concentrated and then lyophilized to get the title compound. Yield: 22% (30.46 mg, white solid). ¹H NMR (400 MHz, DMSO-d₆): δ 12.02 (s, 1H), 9.12 (s, 1H), 8.59 (s, 1H), 8.42 (d, J=7.6 Hz, 1H), 7.95 (s, 1H), 7.60 (d, J=3.6 Hz, 1H), 6.75 (d, J=3.2 Hz, 1H), 3.88-3.81 (m, 1H), 3.57-3.52 (m, 2H), 3.45-3.42 (m, 2H), 3.28-3.23 (m, 4H), 2.06-2.03 (m, 2H), 1.97-1.94 (m, 2H), 1.48-1.39 (m, 2H), 1.32-1.23 (m, 2H). LCMS: (Method A) 401.1 (M+H)

### Example 59: 5-(1H-imidazol-1-yl)-N-((1r,4r)-4-(2-methoxyethoxy)cyclohexyl)-1H-pyrrolo[2,3-c]pyridine-7-carboxamide

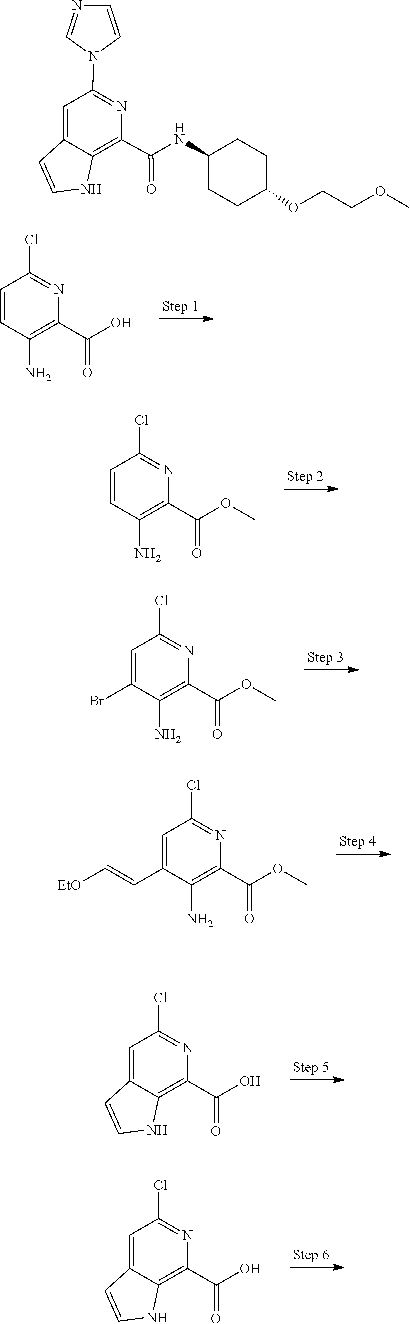

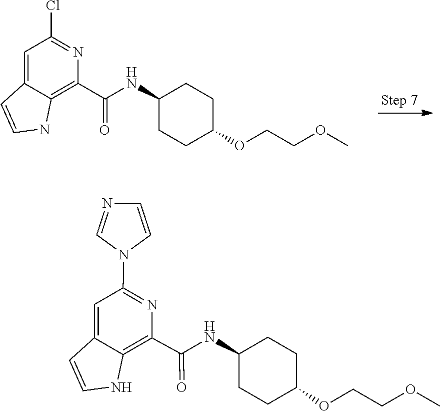 [SMILES: COCCO[C@H]1CC[C@H](NC(=O)c2nc(Cl)cc3cc[nH]c23)CC1 ; COCCO[C@H]1CC[C@H](NC(=O)c2nc(-n3ccnc3)cc3cc[nH]c23)CC1]

### Step 1: Methyl 3-amino-6-chloropicolinate

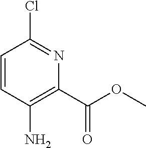

To a stirred solution of 3-amino-6-chloropicolinic acid (10.1 g, 58.52 mmol) in MeOH (150 mL) at 0° C., SOCl₂ (5.51 mL, 76.08 mmol) was added and the reaction mixture was stirred at 75° C. for 16 h. The reaction mixture was monitored by TLC. After complete consumption of the starting material, the reaction mixture was concentrated under vacuum. The residue was dissolved in 10% MeOH in DCM (500 mL) and washed with 10% aq. NaHCO₃ solution (200 mL). The organic layer was dried over anhydrous Na₂SO₄ and concentrated under vacuum to get the title compound. Yield: 92% (10.1 g, yellow solid). ¹H NMR (300 MHz, DMSO-d₆): 7.38-7.29 (m, 2H), 6.89 (s, 2H), 3.81 (m, 3H). LCMS: (Method C) 187.0 (M+H).

### Step 2: methyl 3-amino-4-bromo-6-chloropicolinate

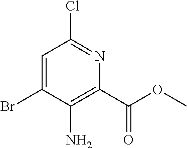

To a stirred solution of methyl 3-amino-6-chloropicolinate (10.09 g, 54.07 mmol) in DMF (100 mL) at 0° C., NBS (11.54 g, 64.88 mmol) was added and the reaction mixture was warmed to RT and stirred at RT for 16 h. After completion, the reaction mixture was poured into ice cold water (500 mL) and was stirred for 20 min. The precipitated solid was filtered and washed with water (2×100 mL) then dried to get the title compound. Yield: 71% (10.20 g, yellow solid). ¹H NMR (300 MHz, DMSO-d₆): δ 7.96 (s, 1H), 6.94 (s, 2H), 3.85 (s, 3H). LCMS: (Method C) 264.9 (M+H).

### Step 3: methyl (E)-3-amino-6-chloro-4-(2-ethoxyvinyl)picolinate

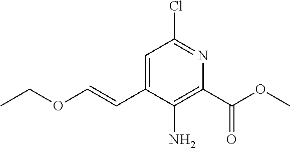 [SMILES: CCO/C=C/c1cc(Cl)nc(C(=O)OC)c1N]

To a stirred solution of methyl 3-amino-4-bromo-6-chloropicolinate (2 g, 9.07 mmol) in a mixture of acetonitrile (30 mL) and water (5 mL), (E)-2-(2-ethoxyvinyl)-4,4,5,5-tetramethyl-1,3,2-dioxaborolane (1.79 g, 9.07 mmol) and K₃PO₄ (3.85 g, 18.15 mmol) were added at RT. Nitrogen gas was purged through the reaction mixture for 10 min before the addition of Pd(OAc)₂ (101 mg, 0.45 mmol) and SPhos (372 mg, 0.90 mmol). The reaction mixture was heated at 70° C. for 16 h. Four batches of this reaction was performed in parallel and were combined before work up. After completion, the reaction mixture was filtered through Celite, and the Celite bed was washed with EtOAc (1 L). The combined filtrate was washed with brine solution (300 mL) followed by water (500 mL). The organic layer was dried over anhydrous Na₂SO₄, filtered and the filtrate was concentrated under vacuum. The resulting crude residue was purified by grace chromatography using silica gel (100-200 mesh, 15-20% EtOAc in pet ether) to get the title compound. Yield: 69% (6.50 g, yellow gummy solid). ¹H NMR (400 MHz, DMSO-d₆): δ 7.46-7.43 (m, 2H), 6.85 (s, 2H), 5.95 (d, J=12.8 Hz, 1H), 4.01 (q, J=7.2 Hz, 2H), 3.81 (s, 3H), 1.28 (t, J=7.2 Hz, 3H). LCMS: (Method C) 257.0 (M+H).

### Step 4: methyl 5-chloro-1H-pyrrolo[2,3-c]pyridine-7-carboxylate

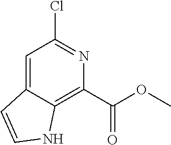

A solution of methyl (E)-3-amino-6-chloro-4-(2-ethoxyvinyl)picolinate (6 g, 6.23 mmol) in acetic acid (30 mL) was stirred for 16 h at 110° C. After completion, the reaction mixture was concentrated under vacuum. The resulting residue was dissolved in EtOAc (1 L) and washed with 10% aq. NaHCO₃ solution (400 mL). The organic layer was washed with brine solution (400 mL) followed by water (400 mL). The organic layer was dried over anhydrous Na₂SO₄, filtered and the filtrate was concentrated under vacuum. The resulting crude residue was purified by grace chromatography using silica gel (100-200 mesh, 15-25% EtOAc in pet ether) to get the title compound. Yield: 67% (3.53 g, yellow solid). ¹H NMR (400 MHz, DMSO-d₆): δ 11.10 (s, 1H), 7.95 (d, J=0.8 Hz, 1H), 7.78-7.76 (m, 1H), 6.66-6.65 (m, 1H), 3.99 (s, 3H). LCMS: (Method C) 211.0 (M+H).

### Step 5: 5-chloro-1H-pyrrolo[2,3-c]pyridine-7-carboxylic acid

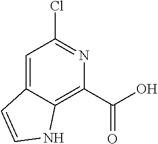 [SMILES: O=C(O)c1nc(Cl)cc2cc[nH]c12]

To a stirred solution methyl 5-chloro-1H-pyrrolo[2,3-c]pyridine-7-carboxylate (1.02 g, 4.84 mmol) in a mixture of THF (5 mL) and methanol (5 mL) at RT, aq. NaOH solution (5 mL, 3 N) was added and the mixture was stirred at RT for 16 h. After completion (the starting material was consumed according to TLC), the reaction mixture was concentrated under vacuum and the residue was diluted with water (10 mL) and then acidified with aq. HCl (10 mL, 1.5 N). The mixture was stirred for 10 min and then extracted with EtOAc (300 mL). The combined organic layer was washed with water (2×100 mL), dried over anhydrous Na₂SO₄, filtered and the filtrate was concentrated under vacuum to get the title compound. Yield: 89% (851 mg, yellow solid). ¹H NMR (400 MHz, DMSO-d₆): 13.59 (s, 1H), 11.70 (s, 1H), 7.91 (s, 1H), 7.72-7.70 (m, 1H), 6.64-6.63 (s, 1H). LCMS: (Method C) 197.0 (M+H).

### Step 6: 5-chloro-N-((1r,4r)-4-(2-methoxyethoxy)cyclohexyl)-1H-pyrrolo[2,3-c]pyridine-7-carboxamide

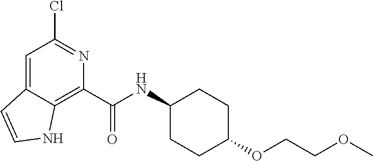 [SMILES: COCCO[C@H]1CC[C@H](NC(=O)c2nc(Cl)cc3cc[nH]c23)CC1]

To a stirred solution of 5-chloro-1H-pyrrolo[2,3-c]pyridine-7-carboxylic acid (845 mg, 4.29 mmol) in DMF (10 mL) at 0° C., HATU (1.11 g, 6.44 mmol) followed by DIPEA (1.98 mL, 10.74 mmol) were added under nitrogen atmosphere and the stirring was continued at 0° C. for 5 min. Then (1r,4r)-4-(2-methoxyethoxy)cyclohexan-1-amine (1.11 g, 6.44 mmol) was added the reaction mixture was stirred for 16 h at RT. After completion (the starting material was consumed according to TLC), the reaction mixture was diluted with water (100 mL) and then extracted with EtOAc (200 mL). The resulting organic solution was washed with water (2×50 mL), dried over anhydrous Na₂SO₄, filtered and the filtrate was concentrated under vacuum. The resulting crude residue was purified by grace chromatography using silica gel (100-200 mesh, 50%-70% EtOAc in pet ether) to get the title compound. Yield: 62% (935 mg, brown solid). LCMS: (Method C) 352.0 (M+H).

### Step 7: 5-(1H-imidazol-1-yl)-N-((1r,4r)-4-(2-methoxyethoxy)cyclohexyl)-1H-pyrrolo[2,3-c]pyridine-7-carboxamide

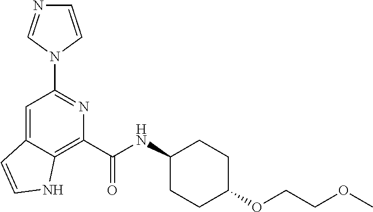 [SMILES: COCCO[C@H]1CC[C@H](NC(=O)c2nc(-n3ccnc3)cc3cc[nH]c23)CC1]

A stirred solution of 5-chloro-N-((1r,4r)-4-(2-methoxyethoxy)cyclohexyl)-1H-pyrrolo[2,3-c]pyridine-7-carboxamide (900 mg, 2.55 mmol) and 1H-imidazole (3.53 g, 51.16 mmol) in DMF (30 mL) at RT was purged with nitrogen gas for 5 min. Then K₂CO₃ (7.07 g, 51.16 mmol) and CuI (243 mg, 1.27 mmol) were added at RT and the reaction mixture was heated at 140° C. for 2 d. After completion, the reaction mixture was filtered through Celite, and the Celite bed was washed with 10% MeOH in DCM (200 mL). The combined organic layer was washed with brine solution (200 mL) followed by water (2×100 mL) and then concentrated under vacuum. The resulting crude residue was purified by Prep HPLC (method B). The prep-fraction was concentrated under reduced pressure, the residue was diluted with 10% MeOH in DCM (100 mL) and washed with 10% aq. NaHCO₃ solution (50 mL) followed by water (2×50 mL). The organic layer was dried over anhydrous Na₂SO₄, filtered, concentrated under reduced pressure and finally lyophilized to get the title compound. Yield: 26% (258.37 mg, off white solid). ¹H NMR (400 MHz, DMSO-d₆): δ 11.70 (s, 1H), 8.84 (d, J=1.2 Hz, 1H), 8.61 (d, J=8.8 Hz, 1H), 8.17-8.16 (m, 1H), 8.13 (s, 1H), 7.71-7.70 (m, 1H), 7.12-7.11 (s, 1H), 6.64-6.63 (m, 1H), 3.93-3.89 (m, 1H), 3.58-3.55 (m, 2H), 3.46-3.43 (m, 2H), 3.33-3.26 (m, 4H), 2.08-2.06 (m, 2H), 1.91-1.89 (m, 2H), 1.67-1.59 (m, 2H), 1.33-1.24 (m, 2H), LCMS: (Method A) 384.3 (M+H).

### Example 60: N-((1r,4r)-4-(2-methoxyethoxy)cyclohexyl)-5-(thiazol-5-yl)-1H-pyrrolo[3,2-b]pyridine-7-carboxamide

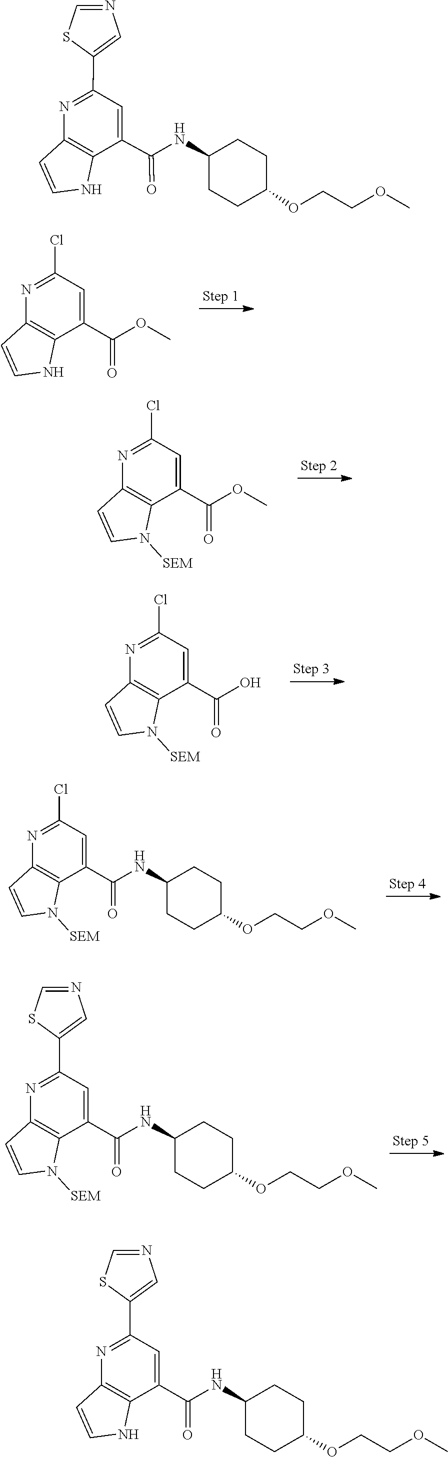

### Step 1: methyl 3-amino-2-bromo-6-chloroisonicotinate

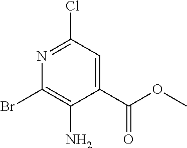

To a stirred solution of 5-amino-2-chloroisonicotinic acid (10.0 g, 57.96 mmol) in methanol (50 mL), was added thionyl chloride (8.4 mL, 115.9 mmol) dropwise at RT and the reaction mixture was stirred at 75° C. for 48 h. After completion, the reaction mixture was concentrated under reduced pressure. The resulting residue was diluted with water and adjusted to slightly basic pH using aq. NaHCO₃. The resulting solid was collected using filtration. The solid was dissolved in hot ethyl acetate, dried over anhydrous Na₂SO₄ and then concentrated under vacuum to afford the ester intermediate. Yield: 69% (7.5 g, yellow solid). To a stirred solution of methyl 5-amino-2-chloroisonicotinate (7.5 g, 40.3 mmol) in DMF (60 mL), was added NBS (8.61 g, 48.3 mmol) at RT and the reaction mixture was stirred at 85° C. for 16 h. After completion, approximately half of the solvent was evaporated under vacuum and the resulting mixture was diluted with ice cold water. The resulting solid was collected using filtration to get the title compound. Yield: 81% (8.7 g, yellow solid). ¹H NMR (400 MHz, DMSO-d₆): δ 7.65-7.63 (m, 1H), 6.80 (s, 2H), 3.87 (s, 3H). LCMS: (Method C) 265.0 (M+H).

### Step 2: methyl (E)-3-amino-6-chloro-2-(2-ethoxyvinyl)isonicotinate

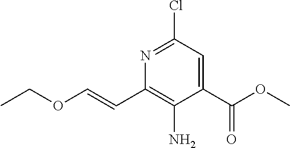 [SMILES: CCO/C=C/c1nc(Cl)cc(C(=O)OC)c1N]

A stirred suspension of methyl 3-amino-2-bromo-6-chloroisonicotinate (3 g, 11.32 mmol) and K₃PO₄ (4.79 g, 22.64 mmol) in a mixture of acetonitrile (40 mL) and water (5 mL) was purged with nitrogen gas for 10 min at RT. Then SPhos (464 mg, 1.13 mmol), Pd(OAc)₂ (127 mg, 0.56 mmol) and (E)-2-(2-ethoxyvinyl)-4,4,5,5-tetramethyl-1,3,2-dioxaborolane (2.24 g, 11.32 mmol) were added at RT and the reaction mixture was heated at 75° C. for 8 h. The reaction mixture was monitored by TLC. After completion, the reaction mixture was quenched with water (150 mL) and the resulting suspension was extracted with EtOAc (3×150 mL). The combined organic layer was washed with water (100 mL), brine (100 mL), dried over anhydrous Na₂SO₄ and concentrated under vacuum. The resulting crude residue was purified by flash chromatography on Biotage Isolera (230-400 mesh silica gel) to afford the title compound. Yield: 59% (1.7 g, off white solid). ¹H NMR (300 MHz, DMSO-d₆): δ 7.54-7.49 (m, 1H), 7.31 (s, 1H), 6.74 (s, 2H), 6.23 (d, J=15.6 Hz, 1H), 4.04-4.01 (m, 2H), 3.84 (s, 3H), 1.28 (t, J=9.6 Hz, 3H). LCMS: (Method C) 257.0 (M+H).

### Step 3: methyl 5-chloro-1H-pyrrolo[3,2-b]pyridine-7-carboxylate

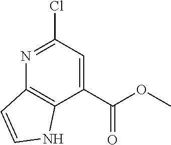

A solution of methyl (E)-3-amino-6-chloro-2-(2-ethoxyvinyl)isonicotinate (3.3 g) in AcOH (30 mL), was stirred at 110° C. for 18 h. After completion, the reaction mixture was cooled and quenched with ice pieces followed by water. It was made slightly basic using aq. NaHCO₃. The resulting solid was collected using filtration to get the title compound. Yield: 48% (1.3 g, yellow solid). ¹H NMR (400 MHz, DMSO-d₆): δ 11.74 (s, 1H), 7.85-7.84 (m, 1H), 7.56 (s, 1H), 6.71-6.69 (m, 1H), 4.00 (s, 3H). LCMS: (Method C) 211.0 (M+H).

### Step 4: methyl 5-chloro-1-((2-(trimethylsilyl)ethoxy)methyl)-JH-pyrrolo[3,2-b]pyridine-7-carboxylate

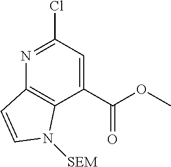 [SMILES: COC(=O)c1cc(Cl)nc2ccn(COCC[Si](C)(C)C)c12]

To a stirred solution of methyl 5-chloro-1H-pyrrolo[3,2-b]pyridine-7-carboxylate (200 mg, 0.95 mmol) in THF (5 mL) at 0° C., sodium hydride (57 mg, 1.42 mmol, 60% in paraffin oil) was added under nitrogen atmosphere with continuous stirring. SEM-Cl (237 mg, 1.42 mmol) was added at the same temperature and then the reaction mixture was stirred at RT for 16 h. After completion, the reaction mixture was quenched with ice cold water (15 mL) and the suspension was extracted with EtOAc (3×30 mL). The combined organic layer was washed with water (10 mL), dried over anhydrous Na₂SO₄, filtered and the filtrate was concentrated under vacuum. The resulting crude product was purified by flash chromatography on Biotage Isolera using silica gel (230-400 mesh) to get the title compound. Yield: 92% (300 mg). LCMS: (Method C) 341.0 (M+H).

### Step 5: 5-chloro-1-((2-(trimethylsilyl)ethoxy)methyl)-JH-pyrrolo[3,2-b]pyridine-7-carboxylic acid

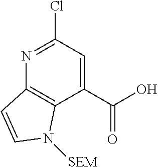 [SMILES: C[Si](C)(C)CCOCn1ccc2nc(Cl)cc(C(=O)O)c21]

To a stirred solution of methyl 5-chloro-1-((2-(trimethylsilyl)ethoxy)methyl)-1H-pyrrolo[3,2-b]pyridine-7-carboxylate (0.9 g, 2.63 mmol) in a mixture of THF (5 mL), methanol (5 mL) and water (5 mL), LiOH.H₂O (1.1 g, 26.39 mmol) was added at RT and the reaction mixture was stirred at RT for 18 h. After completion, the reaction mixture was concentrated and the residue was dissolved in water and made slightly acidic using aq. HCl (1.5 N). The resulting solid was collected using filtration and dried to get the title compound. Yield: 83% (0.72 g, yellow solid). ¹H NMR (400 MHz, DMSO-d₆): δ 7.96-7.95 (m, 1H), 7.35 (s, 1H), 6.69-6.68 (m, 1H), 5.77 (s, 2H), 3.26-3.21 (m, 2H), 0.72 (t, J=8.0 Hz, 2H), −0.15 (s, 9H). LCMS: (Method C) 327.2 (M+H).

### Step 6: 5-chloro-N-((1r,4r)-4-(2-methoxyethoxy)cyclohexyl)-1-((2-(trimethylsilyl)ethoxy)methyl)-1H-pyrrolo[3,2-b]pyridine-7-carboxamide

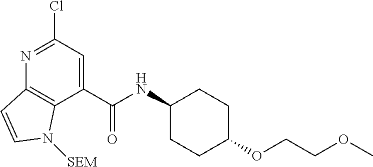 [SMILES: COCCO[C@H]1CC[C@H](NC(=O)c2cc(Cl)nc3ccn(COCC[Si](C)(C)C)c23)CC1]

To a stirred solution of 5-chloro-1-((2-(trimethylsilyl)ethoxy)methyl)-1H-pyrrolo[3,2-b]pyridine-7-carboxylic acid (0.71 g, 2.17 mmol) in DMF (5 mL) were added DIPEA (0.84 g, 6.53 mmol), EDC.HCl (0.62 g, 3.26 mmol) and HOBt (0.44 g, 3.26 mmol) at RT. After stirring for 5 min, a solutions of (1r,4r)-4-(2-methoxyethoxy)cyclohexan-1-amine (0.45 g, 2.61 mmol) was added and the reaction mixture was stirred at RT for 18 h. After completion (monitored by TLC) the reaction mixture was diluted with water (30 mL) and the resulting suspension was extracted with EtOAc (3×100 mL). The combined organic layer was washed with water (50 mL), brine (50 mL), dried over anhydrous Na₂SO₄ and the solvent was evaporated under vacuum to afford the title compound. Yield: 54% (0.41 g, yellow solid). ¹H NMR (400 MHz, DMSO-d₆): δ 8.69 (d, J=7.6 Hz, 1H), 7.94 (d, J=3.2 Hz, 1H), 7.16 (s, 1H), 6.67 (d, J=3.2 Hz, 1H), 5.59 (s, 2H), 3.77-3.71 (m, 1H), 3.55-3.52 (m, 2H), 3.44-3.41 (m, 2H), 3.28-3.23 (m, 6H), 2.02-1.94 (m, 4H), 1.35-1.23 (m, 4H), 0.74 (t, J=8.4 Hz, 2H), −0.09 (s, 9H). LCMS: (Method C) 482.2 (M+H).

### Step 7: N-((1r,4r)-4-(2-methoxyethoxy)cyclohexyl)-5-(thiazol-5-yl)-1-((2-(trimethylsilyl)ethoxy)methyl)-1H-pyrrolo[3,2-b]pyridine-7-carboxamide

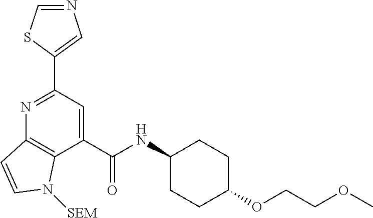 [SMILES: COCCO[C@H]1CC[C@H](NC(=O)c2cc(-c3cncs3)nc3ccn(COCC[Si](C)(C)C)c23)CC1]

A solution of 5-chloro-N-((1r,4r)-4-(2-methoxyethoxy)cyclohexyl)-1-((2-(trimethylsilyl)ethoxy)methyl)-1H-pyrrolo[3,2-b]pyridine-7-carboxamide (150 mg, 0.31 mmol), in 1,4-dioxane (8 mL) and water (2 mL) was purged with nitrogen and then 5-(4,4,5,5-tetramethyl-1,3,2-dioxaborolan-2-yl)thiazole (99 mg, 0.46 mmol), copper iodide (6 mg, 0.03 mmol), Pd(dppf)Cl₂.DCM complex and K₂CO₃ (129 mg, 0.93 mmol) were added. The reaction mixture was heated at 95° C. for 18 h. The reaction was monitored by TLC. The reaction mixture was diluted with 5% methanol in DCM (50 mL) and then washed with water (2×15 mL). The organic layer was separated and dried over anhydrous Na₂SO₄ and the solvent was evaporated under vacuum. The resulting crude residue was purified by flash chromatography on Biotage Isolera using silica gel (230-400 mesh) to get the title compound. Yield: 72% (120 mg, off white solid). ¹H NMR (400 MHz, DMSO-d₆): δ 9.12 (br s, 1H), 8.71-8.55 (m, 2H), 7.91 (br s, 1H), 7.75 (s, 1H), 6.73 (br s, 1H), 5.61 (s, 2H), 3.95 (br s, 2H), 3.87-3.75 (m, 1H), 3.59-3.41 (m, 4H), 3.28-3.23 (m, 4H), 2.09-1.97 (m, 4H), 1.45-1.29 (m, 4H), 0.81-0.74 (m, 2H), −0.07 (br s, 9H). LCMS: (Method C) 531.3 (M+H).

### Step 8: N-((1r,4r)-4-(2-methoxyethoxy)cyclohexyl)-5-(thiazol-5-yl)-1H-pyrrolo[3,2-b]pyridine-7-carboxamide

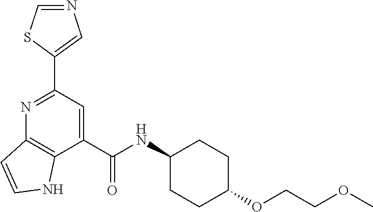 [SMILES: COCCO[C@H]1CC[C@H](NC(=O)c2cc(-c3cncs3)nc3cc[nH]c23)CC1]

A solution of N-((1r,4r)-4-(2-methoxyethoxy)cyclohexyl)-5-(thiazol-5-yl)-1-((2-(trimethylsilyl)ethoxy)methyl)-1H-pyrrolo[3,2-b]pyridine-7-carboxamide (130 mg) in 20% TFA in DCM (3 mL) was stirred at RT for 18 h. After completion (the reaction was monitored by TLC), the reaction mixture was quenched with aq. NaHCO₃ and then extracted with 5% methanol in DCM (2×50 mL). The combined organic layer was dried over anhydrous Na₂SO₄ and the solvent was evaporated under vacuum. The resulting crude residue was purified by prep HPLC (method A). The fraction obtained from prep HPLC was concentrated to remove acetonitrile. The residual water layer was made slightly basic using aq. NaHCO₃. The resulting suspension was extracted using 5% methanol in DCM. The combined organic layer was washed with water, concentrated under reduced pressure and then lyophilized to get the title compound. Yield: 43% (42 mg, white solid). ¹H NMR (400 MHz, DMSO-d₆): δ 11.49 (s, 1H), 9.10 (s, 1H), 8.73 (br s, 1H), 8.52 (s, 1H), 8.14 (s, 1H), 7.67 (d, J=3.2 Hz, 1H), 6.64 (d, J=3.2 Hz, 1H), 3.92-3.83 (m, 1H), 3.58-3.55 (m, 2H), 3.45-3.42 (m, 2H), 3.31-3.26 (m, 4H), 2.09-2.06 (m, 2H), 1.99-1.97 (m, 2H), 1.50-1.41 (m, 2H), 1.33-1.23 (m, 2H). LCMS: (Method C) 401.2 (M+H).

### Example 61 Assay Protocol for Determination of IC50 Against Human CD38

CD38, a differentiation antigen of B lymphocytes, is a type II integral membrane protein. It is also known as ADP-ribosyl cyclase and nicotinamide adenine dinucleotide (NAD) glycohydrolase. Through its production of cyclic ADP-ribose, CD38 modulates calcium-mediated signal transduction in various cells, including pancreatic cells. The major enzymatic activity of CD38 is the hydrolysis of NAD. CD38 is a prognostic biomarker for acute B lymphoblastic leukemia.

The CD38 Inhibitor Screening Assay Kit (BPS Bioscience, Catalog #: 79287) is designed to measure the glycohydrolase activity of CD38 for screening and profiling applications.

CD38 hydrolase buffer (4×) was thawed on ice and 1×CD38 hydrolase buffer was prepared by diluting 4×CD38 hydrolase buffer with water. CD38 hydrolase enzyme was diluted to 0.5 ng/ml with 1×CD38 hydrolase buffer (10 ng/well). Master mix was prepared by mixing equal volumes of water and CD38 hydrolase buffer (4×) and 10 μl of the mix was added to all the wells. Following master mix addition, 10 μl of the inhibitors and 20 μl of CD38 hydrolase enzyme were added to the wells. For wells designated blank, 10 μl and 20 μl of 1×CD38 hydrolase buffer was added respectively in place of CD38 enzyme and inhibitors. The plate was covered and incubated 30 min at room temperature with slow shaking at 400 rpm. After 30 min incubation, the plate was removed and 10 μl of diluted ε-NAD substrate was added to all the wells. The plate is incubated for 10 min, spun at 1200 rpm for 30 secs to remove bubbles and read in Tecan Spark using a fluorometer setting of excitation 300 nm and emission 410 nm. The reading for the blank was subtracted from all the values. IC50 for the test compounds were determined by using Graphpad prism software v7.0.

| | | hCD38 IC₅₀ |
|---|---|---|
| Example No. | Structure | (nM) |
| Example 1 | 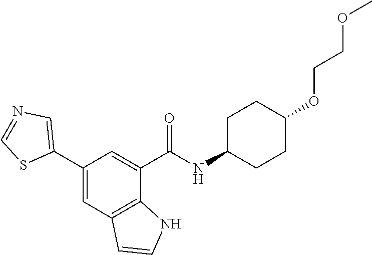 [SMILES: COCCO[C@H]1CC[C@H](NC(=O)c2cc(-c3cncs3)cc3cc[nH]c23)CC1] | 12.8 |
| Example 2 | 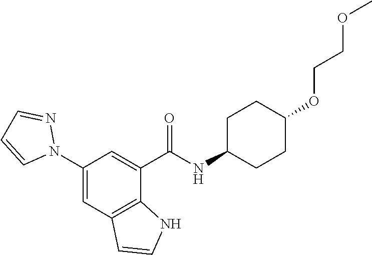 [SMILES: COCCO[C@H]1CC[C@H](NC(=O)c2cc(-n3cccn3)cc3cc[nH]c23)CC1] | >100,000 |
| Example 3 | 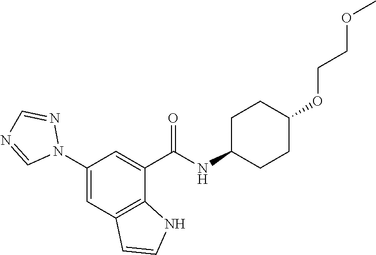 [SMILES: COCCO[C@H]1CC[C@H](NC(=O)c2cc(-n3cncn3)cc3cc[nH]c23)CC1] | 73,000 |
| Example 4 | 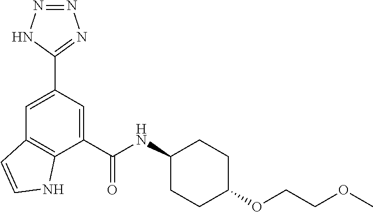 [SMILES: COCCO[C@H]1CC[C@H](NC(=O)c2cc(-c3nnn[nH]3)cc3cc[nH]c23)CC1] | 3,400 |
| Example 5 | 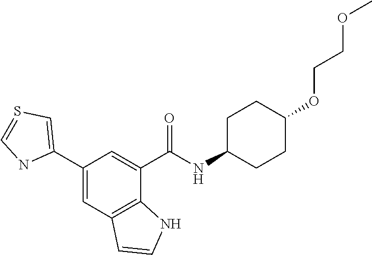 | >100,000 |
| Example 6 | 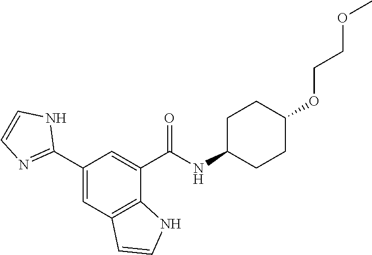 [SMILES: COCCO[C@H]1CC[C@H](NC(=O)c2cc(-c3ncc[nH]3)cc3cc[nH]c23)CC1] | >100,000 |
| Example 7 | 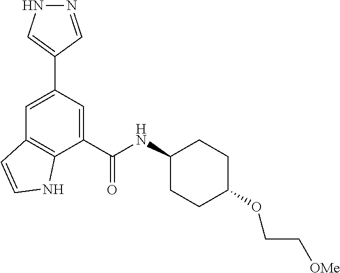 [SMILES: COCCO[C@H]1CC[C@H](NC(=O)c2cc(-c3cn[nH]c3)cc3cc[nH]c23)CC1] | 5,700 |
| Example 8 | 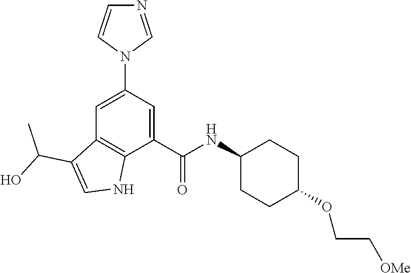 [SMILES: COCCO[C@H]1CC[C@H](NC(=O)c2cc(-n3ccnc3)cc3c(C(C)O)c[nH]c23)CC1] | 17 |
| Examples 9 and 10 | 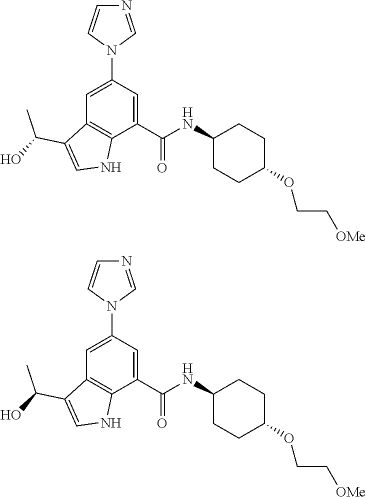 [SMILES: COCCO[C@H]1CC[C@H](NC(=O)c2cc(-n3ccnc3)cc3c([C@@H](C)O)c[nH]c23)CC1 ; COCCO[C@H]1CC[C@H](NC(=O)c2cc(-n3ccnc3)cc3c([C@H](C)O)c[nH]c23)CC1] | |
| | First Eluting Peak | 26 |
| | Second Eluting Peak | 55 |
| Ex 11 | 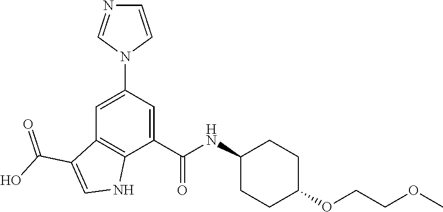 [SMILES: COCCO[C@H]1CC[C@H](NC(=O)c2cc(-n3ccnc3)cc3c(C(=O)O)c[nH]c23)CC1] | 580 |
| Example 12 | 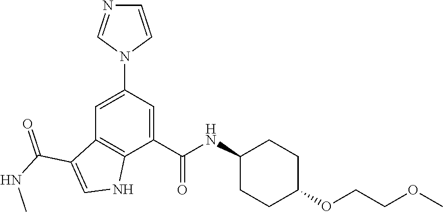 [SMILES: CNC(=O)c1c[nH]c2c(C(=O)N[C@H]3CC[C@H](OCCOC)CC3)cc(-n3ccnc3)cc12] | 56 |
| Example 13 | 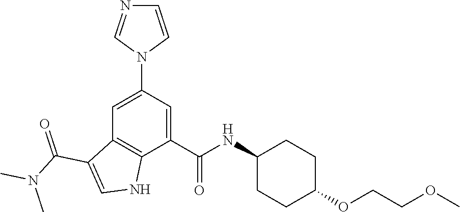 | 103 |
| Example 14 | 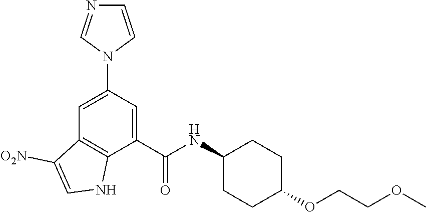 [SMILES: COCCO[C@H]1CC[C@H](NC(=O)c2cc(-n3ccnc3)cc3c([N+](=O)[O-])c[nH]c23)CC1] | 7 |
| Example 15 | 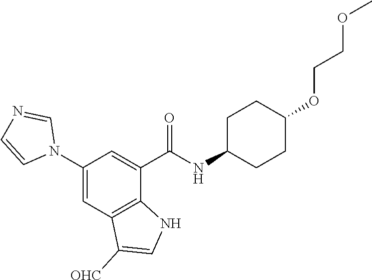 [SMILES: COCCO[C@H]1CC[C@H](NC(=O)c2cc(-n3ccnc3)cc3c(C=O)c[nH]c23)CC1] | 18,000 |
| Example 16 | 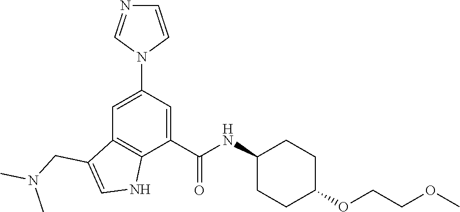 | 800 |
| Example 17 | 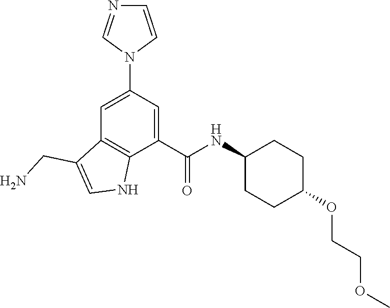 [SMILES: COCCO[C@H]1CC[C@H](NC(=O)c2cc(-n3ccnc3)cc3c(CN)c[nH]c23)CC1] | 890 |
| Example 18 | 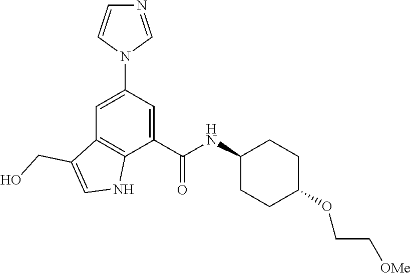 [SMILES: COCCO[C@H]1CC[C@H](NC(=O)c2cc(-n3ccnc3)cc3c(CO)c[nH]c23)CC1] | 34 |
| Example 19 | 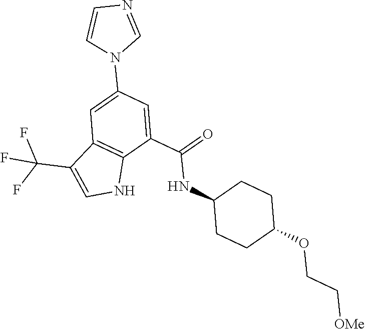 [SMILES: COCCO[C@H]1CC[C@H](NC(=O)c2cc(-n3ccnc3)cc3c(C(F)(F)F)c[nH]c23)CC1] | 41 |
| Example 20 | 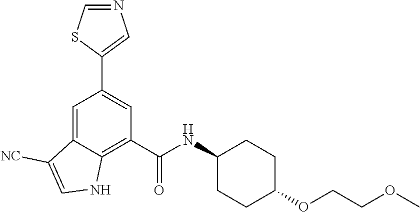 [SMILES: COCCO[C@H]1CC[C@H](NC(=O)c2cc(-c3cncs3)cc3c(C#N)c[nH]c23)CC1] | 19 |
| Example 21 | 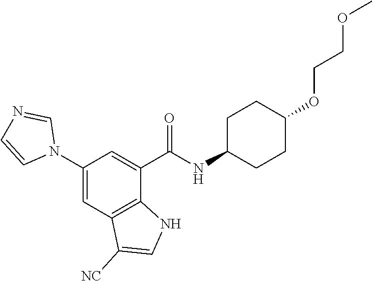 [SMILES: COCCO[C@H]1CC[C@H](NC(=O)c2cc(-n3ccnc3)cc3c(C#N)c[nH]c23)CC1] | 19 |
| Example 22 | 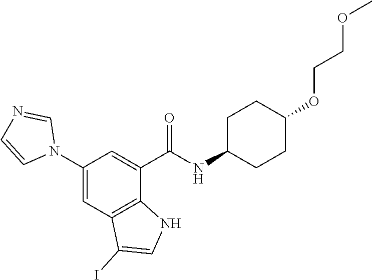 [SMILES: COCCO[C@H]1CC[C@H](NC(=O)c2cc(-n3ccnc3)cc3c(I)c[nH]c23)CC1] | 169 |
| Example 23 | 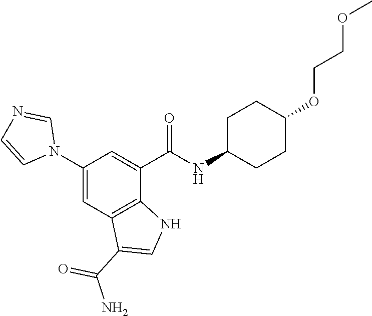 [SMILES: COCCO[C@H]1CC[C@H](NC(=O)c2cc(-n3ccnc3)cc3c(C(N)=O)c[nH]c23)CC1] | 47 |
| Example 24 | 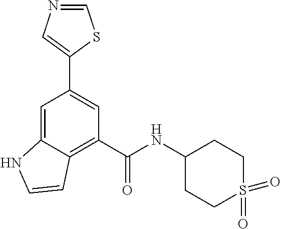 [SMILES: O=C(NC1CCS(=O)(=O)CC1)c1cc(-c2cncs2)cc2[nH]ccc12] | 198 |
| Example 25 | 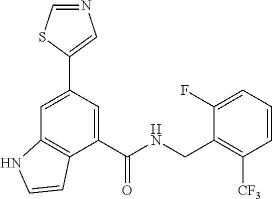 [SMILES: O=C(NCc1c(F)cccc1C(F)(F)F)c1cc(-c2cncs2)cc2[nH]ccc12] | 78 |
| Example 26 | 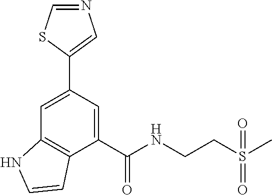 [SMILES: CS(=O)(=O)CCNC(=O)c1cc(-c2cncs2)cc2[nH]ccc12] | 1,000 |
| Example 27 | 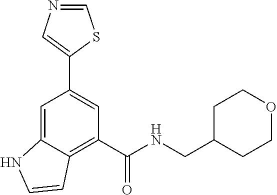 [SMILES: O=C(NCC1CCOCC1)c1cc(-c2cncs2)cc2[nH]ccc12] | 3,700 |
| Example 28 | 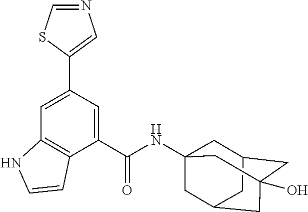 [SMILES: O=C(NC12CC3CC(CC(O)(C3)C1)C2)c1cc(-c2cncs2)cc2[nH]ccc12] | 523 |
| Example 29 | 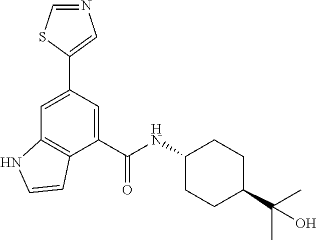 [SMILES: CC(C)(O)[C@H]1CC[C@H](NC(=O)c2cc(-c3cncs3)cc3[nH]ccc23)CC1] | 123 |
| Example 30 | 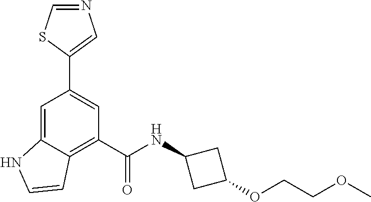 [SMILES: COCCO[C@H]1C[C@H](NC(=O)c2cc(-c3cncs3)cc3[nH]ccc23)C1] | 761 |
| Example 31 | 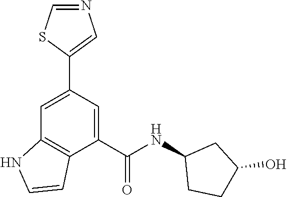 [SMILES: O=C(N[C@@H]1CC[C@@H](O)C1)c1cc(-c2cncs2)cc2[nH]ccc12] | 392 |
| Example 32 | 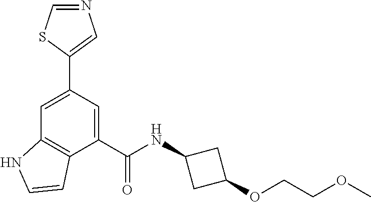 [SMILES: COCCO[C@H]1C[C@@H](NC(=O)c2cc(-c3cncs3)cc3[nH]ccc23)C1] | 3,300 |
| Example 33 | 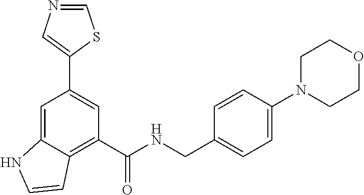 [SMILES: O=C(NCc1ccc(N2CCOCC2)cc1)c1cc(-c2cncs2)cc2[nH]ccc12] | >10,000 |
| Example 34 | 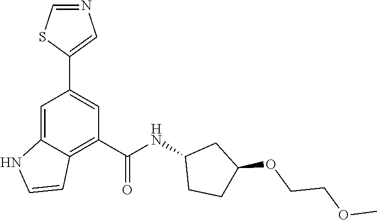 [SMILES: COCCO[C@H]1CC[C@H](NC(=O)c2cc(-c3cncs3)cc3[nH]ccc23)C1] | 265 |
| Example 35 | 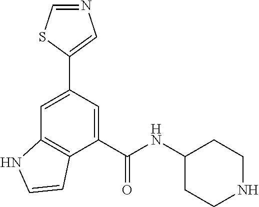 [SMILES: O=C(NC1CCNCC1)c1cc(-c2cncs2)cc2[nH]ccc12] | 7,800 |
| Example 36 | 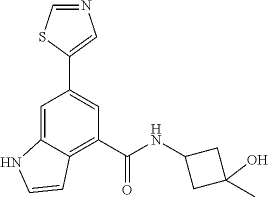 [SMILES: CC1(O)CC(NC(=O)c2cc(-c3cncs3)cc3[nH]ccc23)C1] | 1,500 |
| Example 37 | 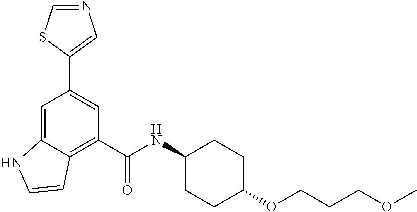 [SMILES: COCCCO[C@H]1CC[C@H](NC(=O)c2cc(-c3cncs3)cc3[nH]ccc23)CC1] | 63 |
| Example 38 | 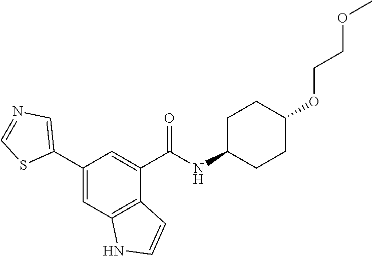 [SMILES: COCCO[C@H]1CC[C@H](NC(=O)c2cc(-c3cncs3)cc3[nH]ccc23)CC1] | 71 |
| Example 39 | 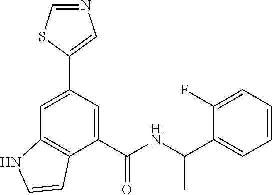 [SMILES: CC(NC(=O)c1cc(-c2cncs2)cc2[nH]ccc12)c1ccccc1F] | 197 |
| Example 40 | 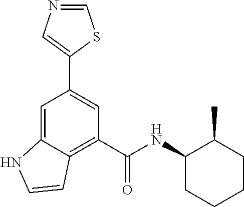 [SMILES: C[C@H]1CCCC[C@H]1NC(=O)c1cc(-c2cncs2)cc2[nH]ccc12] | 78 |
| Example 41 | 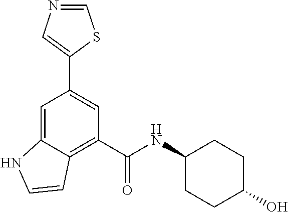 [SMILES: O=C(N[C@H]1CC[C@H](O)CC1)c1cc(-c2cncs2)cc2[nH]ccc12] | 37 |
| Example 42 | 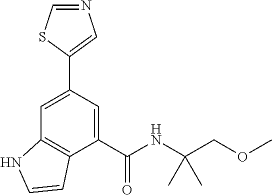 [SMILES: COCC(C)(C)NC(=O)c1cc(-c2cncs2)cc2[nH]ccc12] | 3,600 |
| Example 43 | 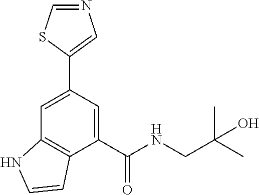 [SMILES: CC(C)(O)CNC(=O)c1cc(-c2cncs2)cc2[nH]ccc12] | 5,100 |
| Example 44 | 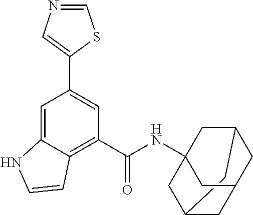 [SMILES: O=C(NC12CC3CC(CC(C3)C1)C2)c1cc(-c2cncs2)cc2[nH]ccc12] | 393 |
| Example 45 | 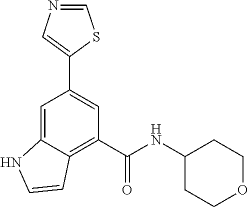 [SMILES: O=C(NC1CCOCC1)c1cc(-c2cncs2)cc2[nH]ccc12] | 664 |
| Example 46 | 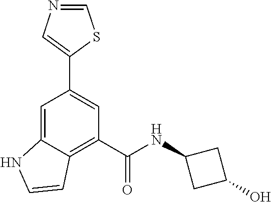 [SMILES: O=C(N[C@H]1C[C@H](O)C1)c1cc(-c2cncs2)cc2[nH]ccc12] | 678 |
| Example 47 | 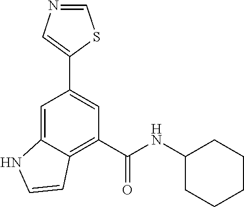 [SMILES: O=C(NC1CCCCC1)c1cc(-c2cncs2)cc2[nH]ccc12] | 138 |
| Example 48 | 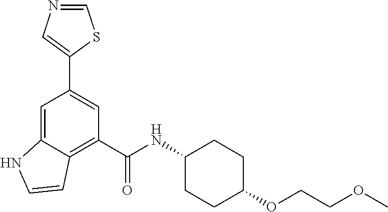 [SMILES: COCCO[C@H]1CC[C@@H](NC(=O)c2cc(-c3cncs3)cc3[nH]ccc23)CC1] | >10,000 |
| Example 49 | 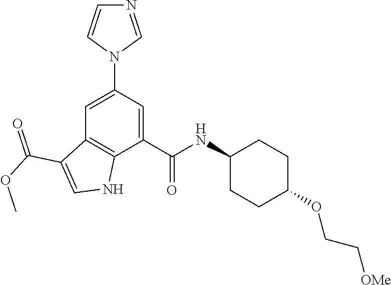 [SMILES: COCCO[C@H]1CC[C@H](NC(=O)c2cc(-n3ccnc3)cc3c(C(=O)OC)c[nH]c23)CC1] | 1,200 |
| Example 50 | 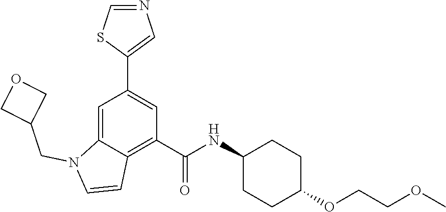 [SMILES: COCCO[C@H]1CC[C@H](NC(=O)c2cc(-c3cncs3)cc3c2ccn3CC2COC2)CC1] | 503 |
| Example 51 | 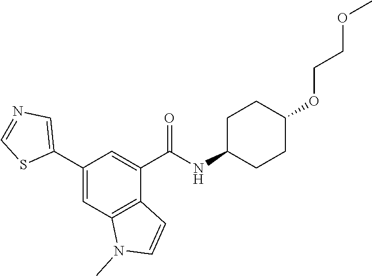 [SMILES: COCCO[C@H]1CC[C@H](NC(=O)c2cc(-c3cncs3)cc3c2ccn3C)CC1] | 94 |
| Example 52 | 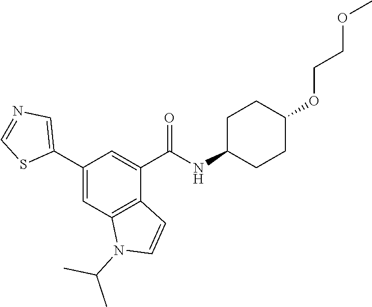 [SMILES: COCCO[C@H]1CC[C@H](NC(=O)c2cc(-c3cncs3)cc3c2ccn3C(C)C)CC1] | 1,400 |
| Example 53 | 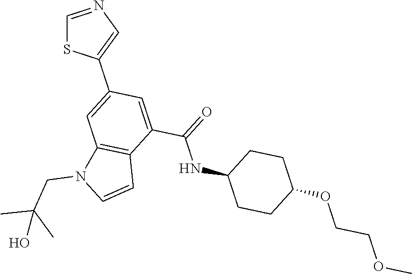 [SMILES: COCCO[C@H]1CC[C@H](NC(=O)c2cc(-c3cncs3)cc3c2ccn3CC(C)(C)O)CC1] | 3,000 |
| Example 54 | 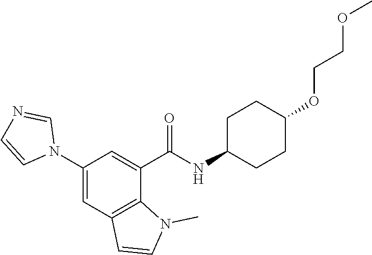 [SMILES: COCCO[C@H]1CC[C@H](NC(=O)c2cc(-n3ccnc3)cc3ccn(C)c23)CC1] | 375 |
| Example 55 | 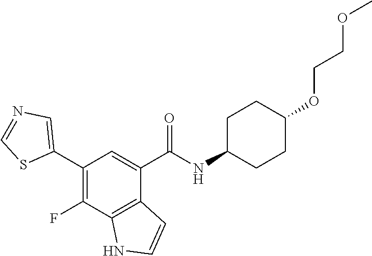 [SMILES: COCCO[C@H]1CC[C@H](NC(=O)c2cc(-c3cncs3)c(F)c3[nH]ccc23)CC1] | 34 |
| Example 56 | 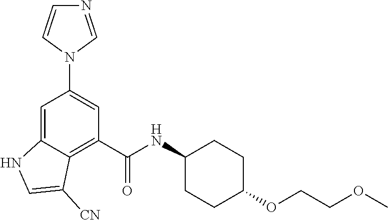 [SMILES: COCCO[C@H]1CC[C@H](NC(=O)c2cc(-n3ccnc3)cc3[nH]cc(C#N)c23)CC1] | 9.8 |
| Example 57 | 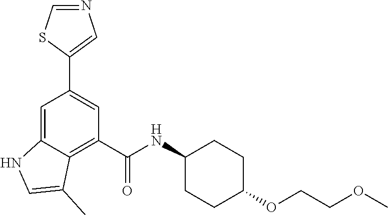 [SMILES: COCCO[C@H]1CC[C@H](NC(=O)c2cc(-c3cncs3)cc3[nH]cc(C)c23)CC1] | 59 |
| Example 58 | 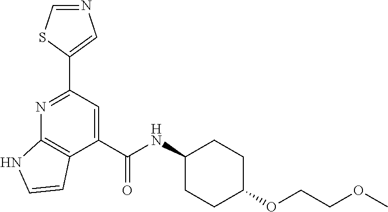 [SMILES: COCCO[C@H]1CC[C@H](NC(=O)c2cc(-c3cncs3)nc3[nH]ccc23)CC1] | 22 |
| Example 59 | 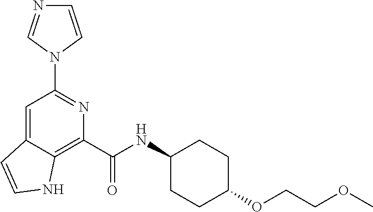 [SMILES: COCCO[C@H]1CC[C@H](NC(=O)c2nc(-n3ccnc3)cc3cc[nH]c23)CC1] | 6 |
| Example 60 | 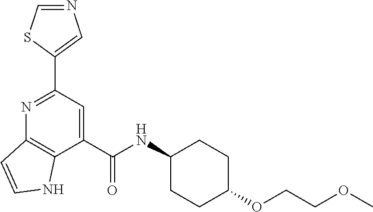 [SMILES: COCCO[C@H]1CC[C@H](NC(=O)c2cc(-c3cncs3)nc3cc[nH]c23)CC1] | 19 |

1. A compound represented by Formula I: 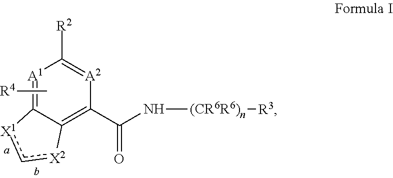
or a pharmaceutically acceptable salt thereof,
wherein:
A¹ and A² are independently CH or N, provided that A¹ and A² are not both N;
X¹ is CR^{1A} and X² is NR^{5A}, when bond a is a double bond and bond b is a single bond; or X¹ is NR^{1B} and X² is CR^{5B}, when bond a is a single bond and bond b is a double bond; and
R^{1A} is H, C₁₋₄alkyl, NO₂, CN, CONR^{a}R^{b}, CH₂NR^{a}R^{b}, (CHR^{c})ₘOH, C₁₋₄haloalkyl, CHO, COOR^{a}, or halo;
R^{a}, R^{b} and R^{c} are each independently H or C₁₋₄alkyl;
R^{1B} is H or C₁₋₄alkyl optionally substituted with 3-5 membered monocyclic heterocyclyl or hydroxy;
R^{5A} is H or C₁₋₄alkyl;
R^{5B} is H, halo, CN, C₁₋₄alkyl, C₁₋₄haloalkyl, NHR^{b} or CONHR^{c};
R² is 5-membered heteroaryl;
R³ is C₁₋₄alkyl, C₃₋₆cycloalkyl, bridged C₇₋₁₂cycloalkyl, 5-6 membered monocyclic heterocyclyl optionally substituted with one or two oxo groups, or phenyl, wherein said alkyl, cycloalkyl, bridged cycloalkyl, heterocyclyl or phenyl is optionally substituted with one or two R^{x} groups, wherein R^{x} is halo, 3 to 6-membered heterocyclyl, C₁₋₄alkyl, C₁₋₄haloalkyl, C₁₋₄hydroxyalkyl, SO₂Me or OR^{d};
R^{d} is H or C₁₋₄alkyl optionally substituted with C₁₋₄alkoxy;
R⁴ is H, halo, CN, C₁₋₄alkyl, C₁₋₄haloalkyl, NHR^{e} or CONHR^{f};
R^{e} and R^{f} are each independently H or C₁₋₄alkyl;
R⁶ is H or C₁₋₄alkyl;
n is 0 or 1; and
m is 1, 2 or 3,
provided that, when R^{1A} is H or C₁₋₄ alkyl; and R² is 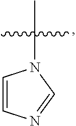 [SMILES: *n1ccnc1]
then n is 1; and
provided that when A¹ is N, X¹ is N and R^{1B} is C₁₋₄ alkyl, then n is 1.

2. The compound of claim 1, represented by Formula II: 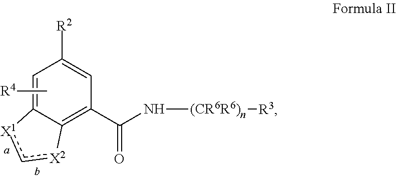
or a pharmaceutically acceptable salt thereof.

3. The compound of claim 1, represented by Formula III or IV: 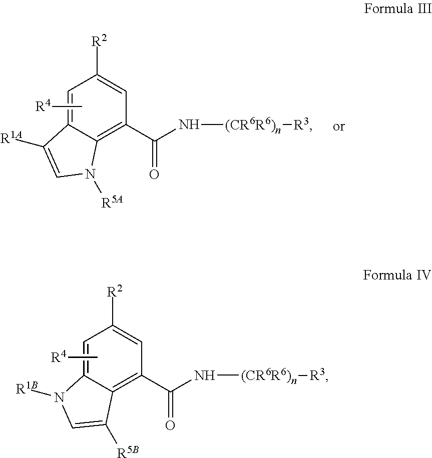
or a pharmaceutically acceptable salt thereof.

4. (canceled)

5. The compound of claim 1, or a pharmaceutically acceptable salt thereof, wherein R² is

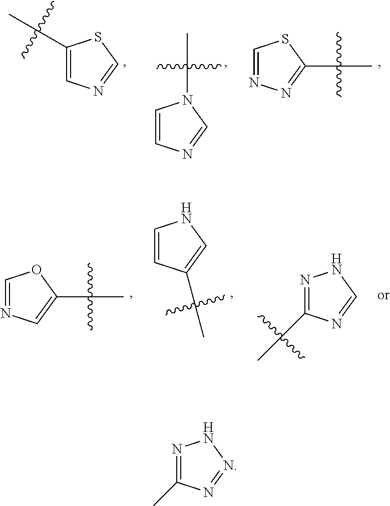

6. The compound of claim 5, or a pharmaceutically acceptable salt thereof, wherein R² is

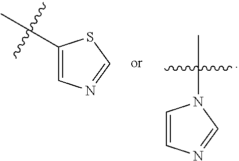 [SMILES: *c1cncs1 ; *n1ccnc1]

7. The compound of claim 1, or a pharmaceutically acceptable salt thereof, wherein R³ is C₃₋₆cycloalkyl; 6-membered monocyclic heterocyclyl optionally substituted with one or two oxo groups; or bridged bicyclic C₉₋₁₁alkyl; wherein said C₃₋₆cycloalkyl, 6-membered monocyclic heterocyclyl or bridged bicyclic C₉₋₁₁alkyl is optionally substituted with one or two R^{x} groups; and n is 0.

8. The compound of claim 7, or a pharmaceutically acceptable salt thereof, wherein R³ is C₃₋₆cycloalkyl or 6-membered monocyclic heterocyclyl optionally substituted with one R^{x} group.

9. The compound of claim 1, wherein the compound is represented by Formula V or VI: 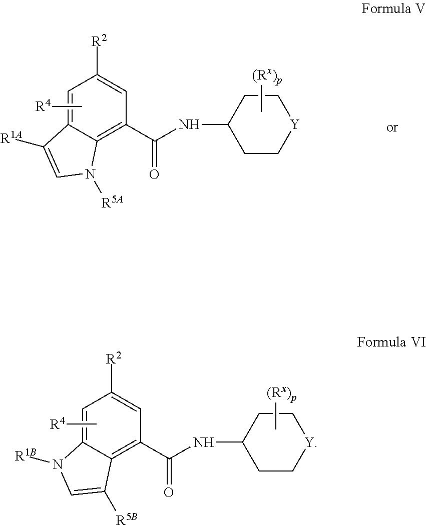
or a pharmaceutically acceptable salt thereof, wherein Y is O, NH, SO₂, CH₂ or CHR^{x}; p is 0 or 1.

10. The compound of claim 9, wherein the compound is represented by Formula VII, VIII, IX, or X: 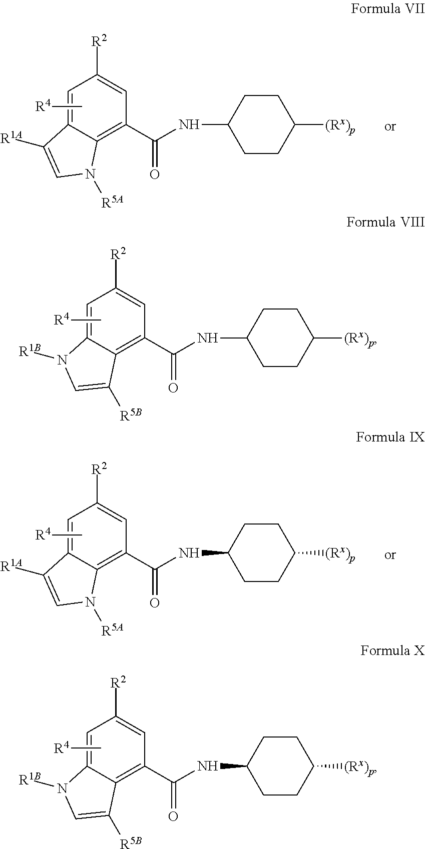
or a pharmaceutically acceptable salt thereof, wherein p is 0 or 1.

11. (canceled)

12. The compound of claim 1, or a pharmaceutically acceptable salt thereof, wherein R^{x} is C₁₋₄hydroxyalkyl or OR^{d}.

13. (canceled)

14. (canceled)

15. The compound of claim 12, or a pharmaceutically acceptable salt thereof, wherein R^{x} is OH, OCH₂CH₂OMe or OCH₂CH₂CH₂OMe.

16. The compound of claim 1, or a pharmaceutically acceptable salt thereof, wherein R³ is tetrahydro-2H-pyran or phenyl; and n is 1, wherein said phenyl is optionally substituted with one or two R^{x} groups.

17. The compound of claim 16, or a pharmaceutically acceptable salt thereof, wherein R³ is phenyl, and R^{x} is halo, C₁₋₄haloalkyl or 6-membered heterocyclyl.

18. The compound of claim 16, wherein the compound is represented by Formula XI or XII: 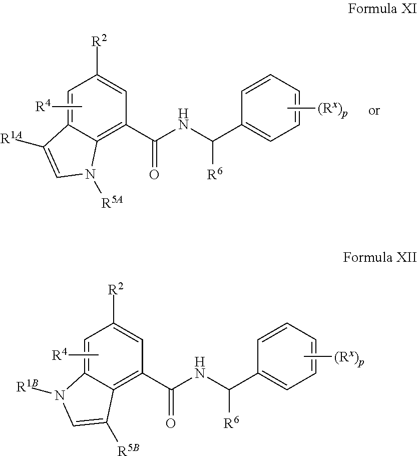
or a pharmaceutically acceptable salt thereof, wherein R^{x} is C₁₋₄haloalkyl, halo or 6-membered heterocyclyl; and wherein p is 1 or 2.

19. The compound of claim 18, or a pharmaceutically acceptable salt thereof, wherein p is 2, and one R^{x} is trifluoromethyl and another R^{x} is F; or wherein p is 1, and R^{x} is F.

20. The compound of claim 16, or a pharmaceutically acceptable salt thereof, wherein R⁶ is H or methyl.

21. The compound of claim 1, or a pharmaceutically acceptable salt thereof, wherein R^{1A} is H, halo, NO₂, CN, COOR^{a}, CHO, CONR^{a}R^{b}, (CHR^{c})ₘOH, CH₂NR^{a}R^{b} or C₁₋₄haloalkyl; or wherein R^{1B} is H or C₁₋₃alkyl optionally substituted with 4-membered heterocyclyl or hydroxyl; and wherein R^{a} and R^{b} are independently H or methyl.

22. (canceled)

23. The compound of claim 21, or a pharmaceutically acceptable salt thereof, wherein R^{1A} is H, I, NO₂, CN, CH₂NH₂, CHO, COOH, COOMe, CH₂OH, CHOHMe, CH₂CH₂OH, CF₃, CONH₂, CONHMe or CONMe₂; or wherein R^{1B} is H, methyl, isopropyl, hydroxyisobutyl or oxetylmethyl.

24. The compound of claim 1, or a pharmaceutically acceptable salt thereof, wherein R⁴ is H or halo; and wherein R^{5A} is H or methyl: or wherein R^{5B} is H, methyl or CN.

25. (canceled)

26. A pharmaceutical composition comprising a pharmaceutically acceptable carrier or excipient and the compound of claim 1, or a pharmaceutically acceptable salt thereof.

27. A method of treating a disease or condition in a subject that benefits from an increase in NAD+, comprising administering to the subject an effective amount of a compound of claim 1, or a pharmaceutically acceptable salt thereof, or a pharmaceutical composition comprising a pharmaceutically acceptable carrier or excipient and the compound of claim 1, or a pharmaceutically acceptable salt thereof.

28. The method of claim 27, wherein the disease or condition is a muscle structure disorder, a neuronal activation disorder, a muscle fatigue disorder, a muscle mass disorder, a metabolic disease, a cancer, a vascular disease, an ocular vascular disease, a muscular eye disease, or a renal disease.

29. The method of claim 28, wherein:
the muscle structure disorder is selected from Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorders, myotonic chondrodystrophy, myotonic dystrophy, myotubular myopathy, nemaline body disease, oculopharyngeal MD, or stress urinary incontinence;
the neuronal activation disorder is selected from amyotrophic lateral sclerosis, Charcot-Marie-Tooth disease, Guillain-Barre syndrome, Lambert-Eaton syndrome, multiple sclerosis, myasthenia gravis, nerve lesion, peripheral neuropathy, spinal muscular atrophy, tardy ulnar nerve palsy, and toxic myoneural disorder;
the muscle fatigue disorder is selected from chronic fatigue syndrome, diabetes (type I or II), glycogen storage disease, fibromyalgia, Friedreich's ataxia, intermittent claudication, lipid storage myopathy, MELAS, mucopolysaccharidosis, Pompe disease, or thyrotoxic myopathy; the muscle mass disorder is cachexia, cartilage degeneration, cerebral palsy, compartment syndrome, critical illness myopathy, inclusion body myositis, polymyositis, muscular atrophy (disuse), sarcopenia, steroid myopathy, and systemic lupus erythematosus;
the beta oxidation disease is selected from systemic carnitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, and riboflavin-responsive disorders of β-oxidation (RR-MADD);
the metabolic disease is selected from hyperlipidemia, dyslipidemia, hyperchlolesterolemia, hypertriglyceridemia, HDL hypocholesterolemia, LDL hypercholesterolemia and/or HLD non-cholesterolemia, VLDL hyperproteinemia, dyslipoproteinemia, apolipoprotein A-I hypoproteinemia, atherosclerosis, disease of arterial sclerosis, disease of cardiovascular systems, cerebrovascular disease, peripheral circulatory disease, metabolic syndrome, syndrome X, obesity, diabetes (type I or II), hyperglycemia, insulin resistance, impaired glucose tolerance, hyperinsulinism, diabetic complication, cardiac insufficiency, cardiac infarction, cardiomyopathy, hypertension, Non-alcoholic fatty liver disease (NAFLD), Nonalcoholic steatohepatitis (NASH), thrombus, Alzheimer disease, neurodegenerative disease, demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, and pancreatitis;
the cancer is selected from colon cancer, cancer of the large intestine, skin cancer, breast cancer, prostate cancer, ovarian cancer, and lung cancer;
the vascular disease is selected from peripheral vascular insufficiency, peripheral vascular disease, intermittent claudication, peripheral vascular disease (PVD), peripheral artery disease (PAD), peripheral artery occlusive disease (PAOD), and peripheral obliterative arteriopathy;
the ocular vascular disease is selected from age-related macular degeneration (AMD), stargardt disease, hypertensive retinopathy, diabetic retinopathy, retinopathy, macular degeneration, retinal haemorrhage, and glaucoma;
the muscular eye disease is selected from strabismus, progressive external ophthalmoplegia, esotropia, exotropia, a disorder of refraction and accommodation, hypermetropia, myopia, astigmatism, anisometropia, presbyopia, a disorders of accommodation, and internal ophthalmoplegia; and
the renal disease is selected from glomerulonephritis, glomerulosclerosis, nephrotic syndrome, hypertensive nephrosclerosis, acute nephritis, recurrent hematuria, persistent hematuria, chronic nephritis, rapidly progressive nephritis, acute renal failure, chronic renal failure, diabetic nephropathy, and Bartter's syndrome.

30. The method of claim 27, wherein the disease or condition is selected from genetic lipodystrophy, non-alcoholic fatty liver disease (NAFLD), non-alcoholic steatohepatitis (NASH), renal ischemia/reperfusion injury (IRI), cardiac ischemia/reperfusion injury, Duchenne & Becker muscular dystrophy, diabetes (type I or type II), obesity, sarcopenia, Alpers's Disease, CPEO-Chronic progressive external ophthalmoplegia, Kearns-Sayra Syndrome (KSS), Leber Hereditary Optic Neuropathy (LHON), MELAS-Mitochondrial myopathy, encephalomyopathy, lactic acidosis, and stroke-like episodes, MERRF-Myoclonic epilepsy and ragged-red fiber disease, NARP-neurogenic muscle weakness, ataxia, and retinitis pigmentosa, Pearson Syndrome, platinum-based chemotherapy induced ototoxicity, Cockayne syndrome, xeroderma pigmentosum A, Wallerian degeneration, and HIV-induced lipodystrophy.

31. (canceled)
